(19) United States
(12) Patent Application Publication
(10) Pub. No.: US 2024/0357928 A1
(43) Pub. Date: Oct. 24, 2024

(54) COMPOUND AND ORGANIC ELECTROLUMINESCENT DEVICE

(71) Applicant: Samsung Electronics Co., Ltd., Suwon-si (KR)

(72) Inventors: Eigo MIYAZAKI, Yokohama-city (JP); Rie SAKURAI, Yokohama-city (JP); Atsushi IMAMURA, Yokohama-city (JP); Mitsunori ITO, Yokohama-city (JP)

(21) Appl. No.: 18/630,198

(22) Filed: Apr. 9, 2024

(30) Foreign Application Priority Data
Apr. 18, 2023 (JP) 2023-067916
Nov. 20, 2023 (KR) 10-2023-0161445

(51) Int. Cl. H10K 85/60; C07D 487/22; C07F 7/08; C09K 11/06; H10K 50/12; H10K 85/40

(52) U.S. Cl. CPC H10K 85/6572; C07D 487/22; C07F 7/0812; C09K 11/06; H10K 85/40; C09K 2211/1018; H10K 50/12

(57) ABSTRACT

A compound represented by Formula (1):

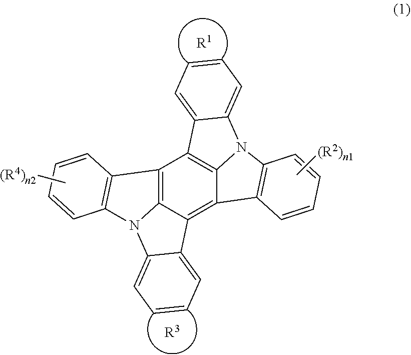

wherein in Formula (1) groups are variables are the same as described in the specification.

### CROSS-REFERENCE TO RELATED APPLICATION

This application is based on and claims priority under 35 U.S.C. § 119 to Korean Patent Application No. 10-2023-0161445, filed on Nov. 20, 2023, in the Korean Intellectual Property Office and Japanese Patent Application No. 2023-067916, filed on Apr. 18, 2023, in the Japanese Patent Office, the content of which are herein incorporated by reference in their entirety.

### BACKGROUND

### 1. Field

The disclosure relates to a compound and an organic electroluminescent device.

### 2. Description of the Related Art

Recently, organic electroluminescent (EL) devices have been used in various light-emitting devices, such as smartphones or televisions. As disclosed by Adachi, Chihaya (ed.), Device properties of organic semiconductors, (Kodansha, 2012), luminescent materials can be used in emission layers of organic EL devices, and fluorescent materials, phosphorescent materials, thermally activated delayed fluorescence materials, and so forth can be used as luminescent materials.

To comply with BT.2100, a new international standard for television broadcasting, light-emitting devices need to exhibit high color purity in the emission wavelength. By introducing a micro-cavity structure in an organic EL device using luminescent materials known in the art, color purity may be improved, and the full width at half maximum of emission wavelength may be narrowed. However, for a light-emitting device with a wide emission spectrum, light deviating from a specific wavelength is not used, which may lead to a decrease in the luminescence efficiency of the device. Accordingly, there remains a demand for a luminescent materials with high color purity and a small full width at half maximum of an emission spectrum.

### SUMMARY

For expanded applications of organic electroluminescent (EL) devices, high efficiency, high luminescence efficiency and long lifespan of device are required. High efficiency and long lifespan of device can be achieved with a derivative of the nitrogen-containing condensed compound (S1).

Additional aspects will be set forth in part in the description which follows and, in part, will be apparent from the description, or may be learned by practice of the presented embodiments of the disclosure.

In an embodiment, a compound represented by Formula (1):

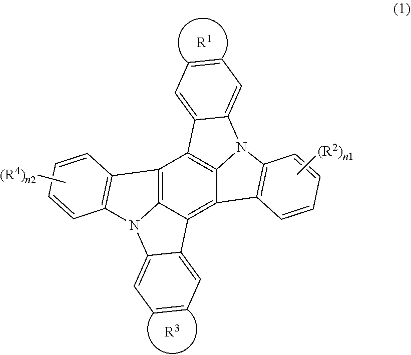

wherein, in Formula (1),
R¹ and R³ are each independently a saturated hydrocarbon group or a saturated heterocyclic group, each including at least one substituent and having 5 to 9 ring-forming atoms,
R² and R⁴ are each independently any one atom or group selected from (a1) to (a8):
(a1) a halogen atom;
(a2) a cyano group;
(a3) a substituted or unsubstituted alkyl group having 1 to 20 carbon atoms;
(a4) a substituted or unsubstituted alkoxy group having 1 to 20 carbon atoms;
(a5) a substituted or unsubstituted arylamino group having 6 to 20 carbon atoms;
(a6) a substituted or unsubstituted triarylsilyl group, a substituted or unsubstituted alkyldiarylsilyl group, a substituted or unsubstituted dialkylarylsilyl group, or a substituted or unsubstituted trialkylsilyl group, wherein an aryl group has 6 to 20 carbon atoms, and an alkyl group has 1 to 20 carbon atoms;
(a7) a substituted or unsubstituted aromatic hydrocarbon group having 6 to 30 carbon atoms; and
(a8) a substituted or unsubstituted heterocyclic group having 3 to 30 ring-forming atoms,
wherein n1 and n2 are each independently 0, 1, 2, 3, or 4, and when n1 is 2 or more, each R² is identical to or different from each other, and when n2 is 2 or more, each R⁴ is identical to or different from each other, and
(a9) when n1 is 2 or more, two R² optionally bond with each other to form a substituted or unsubstituted saturated hydrocarbon group or saturated heterocyclic group having 5 to 9 ring-forming atoms, and when n2 is 2 or more, two R⁴ optionally bond with each other to form a substituted or unsubstituted saturated hydrocarbon group or saturated heterocyclic group having 5 to 9 ring-forming atoms.

In another embodiment, the compound represented by Formula (1), wherein R¹ and R³ have a structure represented by Formula (2):

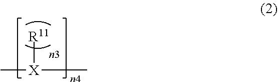

wherein, in Formula (2),
R¹¹ are each independently any one atom or group selected from (b1) to (b7):
(b1) a hydrogen atom or a deuterium atom;
(b2) a cyano group;
(b3) a substituted or unsubstituted alkyl group having 1 to 20 carbon atoms;
(b4) a substituted or unsubstituted alkoxy group having 1 to 20 carbon atoms;
(b5) a substituted or unsubstituted arylamino group having 6 to 20 carbon atoms;
(b6) a substituted or unsubstituted aromatic hydrocarbon group having 6 to 30 carbon atoms; and
(b7) a substituted or unsubstituted heterocyclic group having 6 to 30 ring-forming atoms,
wherein at least one of R¹¹ is selected from (b2) to (b7),
wherein at least one of X is a carbon atom, X are each independently be a carbon atom, a silicon atom, a nitrogen atom, an oxygen atom, a sulfur atom, or a selenium atom, and a plurality of X are identical to or different from each other, and
wherein n3 are each independently be 0, 1, or 2, and when n3 is 2, R¹¹ are identical to or different from each other, and
n4 is 3, 4, 5, 6, or 7.

In an embodiment, an organic EL device includes an emission layer that has a compound with a structure represented by Formula (1).

In another embodiment, the organic EL device includes an emission layer that has a compound with a structure represented by Formula (1) and a phosphorescent complex.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic cross-sectional view illustrating an organic electroluminescent (EL) device according to an embodiment;

FIG. 2 is a schematic cross-sectional view illustrating an organic EL device according to an embodiment;

FIG. 3 is a schematic cross-sectional view illustrating an organic EL device according to an embodiment;

FIG. 4 is an energy level diagram illustrating energy relationships;

FIG. 5 is a graph illustrating photoluminescence intensity (arbitrary units) versus wavelength (nanometers) of a toluene solution of Compound 1 and a toluene solution of Comparative Compound C in accordance with the Examples; and

FIG. 6 is a graph illustrating photoluminescence intensity (arbitrary units) versus wavelength (nanometers) of a thin film formed by co-depositing a host compound and Compound 1 or Comparative Compound C in accordance with the Examples.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Hereinafter, embodiments of the disclosure will be described. However, the disclosure is not limited to the following embodiments. In addition, unless otherwise specified, measurements of operation and physical properties are performed at room temperature (about 20° C. to about 25° C.) and at relative humidity of about 40% RH to about 50% RH.

As used herein, the expression “X and Y are each independently” refers to X and Y being identical to or different from each other.

As used herein, the term “group derived from a ring” refers to a group with free valence by removing hydrogen atoms directly bonded to ring-forming atoms in a ring structure as much as a valence number. Here, ring-forming atoms represent atoms that directly form a ring structure. For example, in the case of a benzene ring, the ring-forming atoms are carbon atoms, and hydrogen atoms are not included in the ring-forming atoms.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, “a”, “an,” “the,” and “at least one” do not denote a limitation of quantity, and are intended to include both the singular and plural, unless the context clearly indicates otherwise. For example, “an element” has the same meaning as “at least one element,” unless the context clearly indicates otherwise. “At least one” is not to be construed as limiting “a” or “an.” “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within +30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

There remains a demand for a luminescent materials with high color purity and a small full width at half maximum of an emission spectrum. Organic EL devices using fluorescent materials which use only fluorescence from a singlet state have been commonly used; however, the luminescence efficiency of such organic EL devices is typically 5% or less. In addition, as organic EL devices using phosphorescent materials typically have luminescence efficiency of 20% or more, such organic EL devices have been widely used for green and red colors. However, for blue color, fluorescence is generally used to ensure adequate device lifespan, and high performance thereof is desirable.

As a method of improving luminescence efficiency while increasing the lifespan of organic EL devices, an organic EL device using an emission method in which a phosphor sensitizer and a luminescent material are combined has been recently suggested (Nature Communications, 2018, 9, 4990). For organic EL devices known in the art, host materials and luminescent materials have been used in an emission layer, and the energy of excitons generated on molecules of the host materials in the emission layer can be transferred to the luminescent materials, thereby emitting light. In this regard, when fluorescent materials are used as the luminescent materials, the luminescence efficiency may be 5%, at the most. When a phosphor sensitizer is added to an emission layer, triplet energy may become available for emission, and accordingly, the luminescence efficiency of organic EL device may be improved to 10% or more. In addition, as it has been reported that the device lifespan increases with a phosphor sensitizer as a luminescent material, accordingly such organic EL devices have gained interest as a candidate for next-generation organic EL devices.

To narrow the full width at half maximum of an emission spectrum, molecule vibrations can be suppressed, and also changes in the conformation or bond lengths of molecules in ground state and in excited state can be suppressed. To suppress conformation changes of molecules, a condensed compound with controlled bond distances between atoms may be preferred. For example, the synthesis and basic properties of the nitrogen-containing condensed polycyclic compound (S1) with a blue color emission wavelength have been previously reported. As disclosed in Tetrahedron. 2013, 69, 3302-3307 and New J. Chem. 2010, 34, 1243-1246, the nitrogen-containing condensed polycyclic compound (S1) maintains an emission spectrum with a small Stokes shift and narrow full width at half maximum in a solution state, and thus is considered a promising luminescent material.

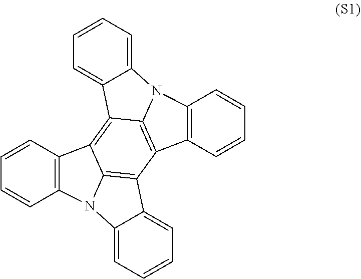 [SMILES: c1ccc2c(c1)c1c3c4ccccc4n4c5ccccc5c(c5c6ccccc6n2c15)c34]

In addition, a derivative with the nitrogen-containing condensed compound (S1) as a basic skeleton has been applied to organic electronic devices (WO 2013/084805). As disclosed in WO 2013/084805, when a derivative of the nitrogen-containing condensed compound (S1) is used as an activation layer of an organic transistor, p-type channel characteristics and high hole mobility are exhibited. In addition, in JP 2020-107742, it is disclosed that a derivative of a nitrogen-containing condensed compound (S1) introducing an aryl group as a substituent functions as a luminescent material of an organic EL device, and the organic EL device exhibits high luminescence efficiency. As such, the nitrogen-containing condensed compound (S1) may be used as a skeleton scaffold for an organic semiconductor material.

Provided is a compound wherein the peak wavelength of the emission spectrum is within the blue wavelength region, to provide high color purity, high luminescence efficiency, and long lifespan. Further provided is an organic EL device with an emission layer including such a compound. Also, provided is a method wherein the peak wavelength of the emission spectrum in the organic EL device is within a blue wavelength region, providing high color purity, high luminescence efficiency, and long lifespan.

It has been found that the aforementioned issued may be overcome with a compound having a particular nitrogen-containing condensed ring structure and by an emission layer including such compound in an organic EL device, and more particularly, by an emission layer including a combination of the compound and a phosphorescent material. The use of such compound is not limited to the foregoing. By including the compound in an emission layer, and more particularly, including the compound along with a phosphorescent material in an emission, the efficiency and lifespan of organic EL device may be improved significantly.

### Compound Represented by Formula (1)

The disclosure relates to a compound represented by Formula (1):

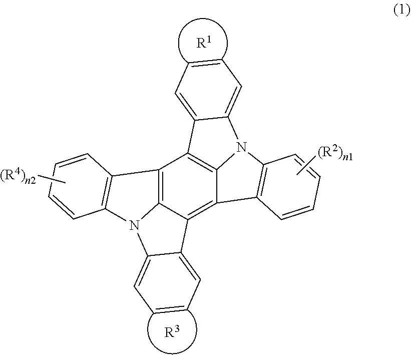

wherein, in Formula (1),
R¹ and R³ may each independently be a saturated hydrocarbon group or a saturated heterocyclic group, each including at least one substituent and having 5 to 9 ring-forming atoms,
R² and R⁴ may each independently be any one atom or group selected from (a1) to (a8):
(a1) a halogen atom;
(a2) a cyano group;
(a3) a substituted or unsubstituted alkyl group having 1 to 20 carbon atoms;
(a4) a substituted or unsubstituted alkoxy group having 1 to 20 carbon atoms;
(a5) a substituted or unsubstituted arylamino group having 6 to 20 carbon atoms;
(a6) a substituted or unsubstituted triarylsilyl group, a substituted or unsubstituted alkyldiarylsilyl group, a substituted or unsubstituted dialkylarylsilyl group, or a substituted or unsubstituted trialkylsilyl group, wherein an aryl group has 6 to 20 carbon atoms, and an alkyl group has 1 to 20 carbon atoms;
(a7) a substituted or unsubstituted aromatic hydrocarbon group having 6 to 30 carbon atoms; and
(a8) a substituted or unsubstituted heterocyclic group having 3 to 30 ring-forming atoms,
wherein n1 and n2 may each independently be 0, 1, 2, 3, or 4, and when n1 is 2 or more, R² may be identical to or different from each other, and when n2 is 2 or more, R⁴ may be identical to or different from each other, and
(a9) when n1 is 2 or more, two R² may optionally bond with each other to form a substituted or unsubstituted saturated hydrocarbon group or saturated heterocyclic group having 5 to 9 ring-forming atoms, and when n2 is 2 or more, two R⁴ may optionally bond with each other to form a substituted or unsubstituted saturated hydrocarbon group or saturated heterocyclic group having 5 to 9 ring-forming atoms.

Hereinafter, the compound represented by Formula (1) may also be referred to as the compound of Formula (1). Moreover, (a9) is described as a group selected as R² and R⁴ along with (a1) to (a8).

While not wishing to be bound by theory, it is understood that an emission wavelength of a luminescence material may not only change by a skeletal structure but also by the type of substituent and bonding position. Due to a particular substituent introduced in a particular position in the compound represented by Formula (1), a peak wavelength of the emission spectrum may become longer to be within a blue wavelength region. As a result, characteristics of luminescent materials of organic electroluminescent (EL) device, more particularly, blue luminescent materials may be sufficiently satisfied. Particularly, aggregation between molecules may be inhibited by the introduction of substituents, the solubility of the molecule itself may be improved, and the degree of purification may be improved, thereby improving luminescence color purity.

For example, in benzene rings located on an outer side of the compound of Formula (1) (four benzene rings in addition to the central benzene ring), there may be four positions to which substituents can be introduced. When a bulky substituent is introduced to one of these benzene rings, adjacent areas of the compound thereof may be sterically protected. For example, when such substitution positions of the benzene ring are defined as first to fourth positions, wherein a position closest to a condensed carbon atom close to a nitrogen atom is the first position, introduction of bulky substituent to the second position or the third position may sterically affect the first and third positions or the second and fourth positions. That is, when a bulky substituent is only introduced to the second position (or the third position), the fourth position (or the first position) may not or hardly be sterically affected. Accordingly, to suppress the Dexter energy transfer effectively, substituents may be introduced to at least two positions of the benzene ring(s) located on an outer side of the compound of Formula (1), and for example, substituents may be introduced to the second and third positions, the first and fourth positions, the first and third positions, or the second and fourth positions. In luminescent materials, to suppress a full width at half maximum (FWHM) of emission spectrum, it is important to decrease conformation changes of molecules and suppress vibrations of molecules. In addition, decreasing conformation changes of molecules to suppress molecular vibrations may prevent degradation of materials. Then, when substituents are introduced to the second and third positions of the four substitution positions, and two substituents are bonded (condensed with a benzene ring), by decreasing the conformation changes due to rotation of substituents, and so forth, vibration of molecules may be suppressed, which leads to improved lifespan of organic EL device. In view of the above considerations, disclosed herein is a structure with a condensed ring (R¹ and R³ in Formula (1)) at a particular position in a benzene ring located on an outer side of the compound of Formula (1).

For compound of Formula (1), a substituent as part of a condensed ring may suppress the Dexter energy transfer, and may decrease the occurrence of decomposition thereof. Accordingly, as degradation of materials in an organic EL device is suppressed, the device lifespan may be improved. In addition, as a ring of R¹ and R³ in Formula (1) has at least one substituent, the spatial volume of the compound of Formula (1) may increase, and accordingly, molecules of the compound of Formula (1) to be spread further apart from one another. As a result, the aggregation among the molecules of the compound of (1) may be inhibited. In general, aggregation among molecules of luminescent materials, may lead to luminescence caused by the aggregation state, and the width of the emission spectrum may increase, thereby degrading color purity. However, as aggregation among molecules of the compound of (1) is decreased, degradation in color purity may not occur, and high-color purity luminescence may be realized. The luminescence efficiency may also be improved as a result. When the concentration of the compound of Formula (1) increases, aggregation may more easily occur; however, for the compound of Formula (1), even when the concentration increases, the aggregation may continue to be suppressed, and luminescence may still be realized with high color purity, high efficiency, and long lifespan. Also, when the compound of Formula (1) is used in combination with the phosphorescent complex, the lifespan of the organic EL device may be significantly prolonged. As such, using the compound of Formula (1) in an organic EL device as a luminescent material facilitates maintained optical characteristics and high luminescence efficiency.

The presumed mechanism is based on assumption and does not affect the technical scope of the disclosure. In addition, regarding other assumptions made herein, the accuracy of such assumptions does not affect the technical scope of the disclosure.

As such, one aspect of the disclosure relates to the compound represented by Formula (1). Another aspect of the disclosure relates to an organic EL device with an emission layer that includes the compound represented by Formula (1). Another aspect of the disclosure relates to an organic EL device with an emission layer that includes the compound represented by Formula (1) and a phosphorescent complex to be described later.

Hereinafter, the compound represented by Formula (1) according to an embodiment and the compound represented by Formula (1) included in the emission layer of the organic EL device according to an embodiment are described.

In Formula (1), R¹ and R³ may each independently be a saturated hydrocarbon group or a saturated heterocyclic group, each including at least one substituent and having 5 to 9 ring-forming atoms. Two of ring-forming atoms in R¹ and R³ may be bonded to a ring-forming carbon atom, to which R¹ or R³ may be bonded, via a single bond. That is, a saturated hydrocarbon group or saturated heterocyclic group having 5 to 9 ring-forming atoms refers to a saturated hydrocarbon cyclic group or saturated heterocyclic group condensed with a ring constituted by ring-forming carbon atoms to which R¹ or R³ may be bonded.

A condensed ring (saturated hydrocarbon ring) formed by a saturated hydrocarbon group having 5 to 9 ring-forming atoms refers to a saturated hydrocarbon ring which does not partially or wholly have an unsaturated bond. Examples of the condensed ring (saturated hydrocarbon ring) formed by a saturated hydrocarbon group having 5 to 9 ring-forming atoms may include a cyclopentane ring, a cyclohexane ring, a cycloheptane ring, a cyclooctane ring, a cyclononane ring, etc.

A condensed ring (saturated heterocyclic ring) formed by a saturated heterocyclic group having 5 to 9 ring-forming atoms refers to a hetero ring which does not partially or wholly have an unsaturated bond. The saturated hetero ring is not particularly limited, and may be, for example, a ring with at least one hetero atom (e.g., a nitrogen atom (N), an oxygen atom (O), a phosphorus atom (P), a sulfur atom(S), or a silicon atom (Si)) as a ring-forming atom, wherein the remaining ring-forming atoms are carbon atoms (C). The hetero atom may be a nitrogen atom (N) or an oxygen atom (O), to provide the desired peak wavelength of the emission spectrum and luminescence color purity.

An atom constituting a ring structure may be bonded to an exocyclic atom via a double bond. For example, a carbon atom constituting the ring structure may constitute a ketone group (CO group), a thioketone group (C═S group), or a C═NH group, or a sulfur atom constituting the ring structure may constitute a sulfinyl group (S═O group) or a sulfonyl group (S(═O)═O group). In this case, as used herein, the exocyclic atom forming the double bond with the atom constituting the ring structure may be part of the hetero ring. Accordingly, a saturated hetero ring may have a ring structure in which an atom constituting the ring structure forms a double bond with an exocyclic atom.

The number of ring-forming heteroatoms in a condensed ring (saturated hetero ring) formed by a saturated heterocyclic group having 5 to 9 ring-forming atoms is not particularly limited; however, the number may be 1 to 3 to provide the desired peak wavelength of the emission spectrum and luminescence color purity.

Examples of the saturated hetero ring may include, but are not particularly limited to, a pyrrolidine ring, tetrahydrofuran ring, a tetrahydrothiophene ring, a piperidine ring, a tetrahydropyran ring, a tetrahydrothiopyran ring, a dioxane ring, a morpholine ring, a dioxolane ring, and the like.

In addition, the term “substituted saturated hydrocarbon group or saturated heterocyclic group having 5 to 9 ring-forming atoms” refers to a group formed by substituting an unsubstituted saturated hydrocarbon group or saturated heterocyclic group with a substituent. Accordingly, when the number of ring-forming atoms in the saturated hydrocarbon group or saturated heterocyclic group is equal to or less than a certain upper limit of the number of ring-forming atoms, for example, 9 or less, the number of ring-forming atoms in the substituted saturated hydrocarbon group or saturated heterocyclic group may be greater than the upper limit.

In Formula (1), at least one substituent of the groups of R¹ and R³ is not particularly limited; however, the at least one substituent may be selected from a halogen atom, a cyano group, a substituted or unsubstituted alkyl group having 1 to 20 carbon atoms, an unsubstituted haloalkyl group having 1 to 20 carbon atoms, an unsubstituted alkoxy group having 1 to 20 carbon atoms, an unsubstituted alkylamino group having 1 to 20 carbon atoms, an unsubstituted arylamino group having 6 to 20 carbon atoms, a substituted or unsubstituted aromatic hydrocarbon group having 6 to 30 carbon atoms, and an unsubstituted heterocyclic group having 3 to 30 ring-forming atoms. A substituent which an alkyl group having 1 to 20 carbon atoms and an aromatic hydrocarbon group having 6 to 30 carbon atoms may have may be a halogen atom, a cyano group, an unsubstituted alkoxy group having 1 to 20 carbon atoms, or an unsubstituted alkyl group having 1 to 20 carbon atoms. The term “substituted alkyl group having 1 to 20 carbon atoms” refers to a group formed by substituting an unsubstituted alkyl group having 1 to 20 carbon atoms with a substituent. Accordingly, the number of carbon atoms in the substituted alkyl group may be greater than 20. The term “substituted aromatic hydrocarbon group having 6 to 30 carbon atoms” refers to a group formed by substituting an unsubstituted aromatic hydrocarbon group having 6 to 30 carbon atoms with a substituent. Accordingly, the number of carbon atoms in the substituted aromatic hydrocarbon group may be greater than 30.

R¹ and R³ may have an additional substituent in addition to the at least one substituent. R¹ and R³ may each have 2, 3, 4, 5, or 6 substituents. R¹ and R³ may each have at least two substituents or at least four substituents. R¹ and R³ may each preferably have four or six substituents. When R¹ and R³ each have at least two substituents, the substituents may be selected from the aforementioned substituents, and the at least two substituents may be substituents of one kind (same substituent) or substituents of two or more kinds (different substituents).

A halogen atom, a cyano group, a substituted or unsubstituted alkyl group having 1 to 20 carbon atoms, an unsubstituted alkoxy group having 1 to 20 carbon atoms, an unsubstituted arylamino group having 6 to 20 carbon atoms, a substituted or unsubstituted aromatic hydrocarbon group having 6 to 30 carbon atoms, and an unsubstituted heterocyclic group having 3 to 30 ring-forming atoms, which are a substituent substituting the groups of R¹ and R³, may be the same as an unsubstituted group described in connection with (a1) to (a9) below.

As for an unsubstituted alkylamino group having 1 to 20 carbon atoms, which is a substituent substituting the groups of R¹ and R³, a nitrogen atom may be bonded via a single bond to at least one atom of the ring-forming atoms forming R¹ and R³ in Formula (1). The alkyl group constituting the alkylamino group is not particularly limited, but may be, for example, the same as described in connection with (a3). The alkylamino group may be, but is not particularly limited to, a monoalkylamino group or a dialkylamino group. Examples of the alkylamino group may include, but are not particularly limited to, an N-methylamino group, an N-ethylamino group, an N-propylamino group, an N-isopropylamino group, an N-butylamino group, an N-isobutylamino group, an N-sec-butylamino group, an N-tert-butylamino group, an N-pentylamino group, an N-hexylamino group, an N,N, N-dimethylamino group, an N-methyl-N-ethylamino group, an N,N-diethylamino group, an N,N-dipropylamino group, an N,N-diisopropylamino group, an N, N-dibutylamino group, an N, N-diisobutylamino group, an N,N-dipentylamino group, and an N, N-dihexylamino group.

An unsubstituted haloalkyl group having 1 to 20 carbon atoms, which is a substituent substituting the groups of R¹ and R³, may be a group in which any one of the ring-forming atoms constituting R¹ and R³ in Formula (1) is substituted with a halogen atom to be described in connection with (a1). The halogen atom may be a fluorine atom to improve device lifespan. Examples of the alkyl group may include a trifluoromethyl group, a trichloromethyl group, a tribromomethyl group, a triiodomethyl group, and the like. In an embodiment, the alkyl group may be a fluoro alkyl group and a trifluoromethyl group.

At least one substituent of the groups of R¹ and R³ may be a halogen atom, a cyano group, an unsubstituted alkyl group having 1 to 20 carbon atoms, an unsubstituted alkoxy group having 1 to 20 carbon atoms, or an unsubstituted arylamino group having 6 to 20 carbon atoms. The at least one substituent of the groups of R¹ and R³ may be an unsubstituted alkyl group having 1 to 20 carbon atoms. Moreover, R¹ and R³ may have an unsubstituted linear or branched alkyl group having 1 to 10 carbon atoms, and may preferably have an unsubstituted linear or branched alkyl group having 1 to 5 carbon atoms. In addition, the at least one substituent of the groups of R¹ and R³ may be a methyl group, an ethyl group, an iso-propyl group, or a tert-butyl group.

In Formula (1), R² and R⁴ may each independently be any one atom or group selected from (a1) to (a9):
(a1) a halogen atom;
(a2) a cyano group;
(a3) a substituted or unsubstituted alkyl group having 1 to 20 carbon atoms;
(a4) a substituted or unsubstituted alkoxy group having 1 to 20 carbon atoms;
(a5) a substituted or unsubstituted arylamino group having 6 to 20 carbon atoms;
(a6) a substituted or unsubstituted triarylsilyl group, a substituted or unsubstituted alkyldiarylsilyl group, a substituted or unsubstituted dialkylarylsilyl group, or a substituted or unsubstituted trialkylsilyl group, wherein an aryl group has 6 to 20 carbon atoms, and an alkyl group has 1 to 20 carbon atoms;
(a7) a substituted or unsubstituted aromatic hydrocarbon group having 6 to 30 carbon atoms;
(a8) a substituted or unsubstituted heterocyclic group having 3 to 30 ring-forming atoms; and
(a9) a substituted or unsubstituted saturated hydrocarbon group or saturated heterocyclic group having 5 to 9 ring-forming atoms and formed by two R² bonded to each other when n1 is 2 or more, and a substituted or unsubstituted saturated hydrocarbon group or saturated heterocyclic group having 5 to 9 ring-forming atoms and formed by two R⁴ bonded to each other when n2 is 2 or more.

In Formula (1), from among the atoms or groups of (a1) to (a9), atoms or groups of (a3), (a6), (a7), (a8), and (a9) may be preferable.

In Formula (1), when the groups of (a3) to (a9) are substituted groups, substituents substituting such groups are not particularly limited. However, in Formula (1), substituents substituting the groups of (a3) to (a9) are each independently selected from a halogen atom, a cyano group, a substituted or unsubstituted alkyl group having 1 to 20 carbon atoms, an unsubstituted haloalkyl group having 1 to 20 carbon atoms, an unsubstituted alkoxy group having 1 to 20 carbon atoms, an unsubstituted alkylamino group having 1 to 20 carbon atoms, an unsubstituted arylamino group having 6 to 20 carbon atoms, a substituted or unsubstituted aromatic hydrocarbon group having 6 to 30 carbon atoms, and an unsubstituted heterocyclic group having 3 to 30 ring-forming atoms.

In Formula (1), when n1 or n2 is 0, corresponding R² or R⁴ is not present. In other words, in Formula (1), when n1 is 0, R² is not present, and when n2 is 0, R⁴ is not present. Thus, in Formula (1), the ring-forming carbon atoms to which R² or R⁴ may be linked are unsubstituted, and hydrogen atoms are linked to the ring-forming carbon atoms.

n1 and n2 may each independently be 0, 1, or 2, and may preferably be 1 or 2. When n1 and n2 are each 1, substitution positions of R² and R⁴ may be the second position or the third position of a benzene ring, and when n1 and n2 are each 2, the substitution positions of R² and R⁴ may be the first and third positions, the second and fourth positions, or the second and the third positions of a benzene ring. When the second and the third positions are substituted, the group of (a9) may be preferable.

The halogen atom of (a1) is not particularly limited, but may be, for example, a fluorine atom, a chlorine atom, a bromine atom, or an iodine atom. In an embodiment, the halogen atom of (a1) may be a fluorine atom to improve device lifespan.

In (a3), the alkyl group having 1 to 20 carbon atoms is not particularly limited, and may be linear, branched, or cyclic. In particular, the alkyl group may be branched to provide the desired device lifespan and luminescence color purity. The number of carbon atoms in the alkyl group may be 2 or more, for example, 3 or more, or for example, 4 or more, to improve solubility and luminescence color purity. In addition, the number of carbon atoms in the alkyl group may be 10 or less, for example, 8 or less, or for example, 6 or less, to improve device lifespan. The number of carbon atoms in the alkyl group may be 4 in the above point of view. Examples of the alkyl group may include, but are not particularly limited to, a methyl group, an ethyl group, an n-propyl group, an isopropyl group, an n-butyl group, an s-butyl group (a sec-butyl group), a t-butyl group (a tert-butyl group), an i-butyl group, a 2-ethyl butyl group, a 3,3-dimethylbutyl group, an n-pentyl group, an i-pentyl group, a neopentyl group, a t-pentyl group, a cyclopentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, a cyclohexyl group, a 4-methylcyclohexyl group, a 4-t-butylcyclohexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, a cyclooctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldodecyl group, a 2-octyldecyl group, an n-tridecyl group, an n-tetradecyl group, an n-pentadecyl group, an n-hexadecyl group, a 2-ethylhexadecyl group, a 2-butylhexadecyl group, an n-heptadecyl group, an n-octadecyl group, an n-nonadecyl group, an n-eicosyl group, and the like. In particular, the alkyl group may be a branched alkyl group, an isopropyl group, or a tert-butyl group, for example, a tert-butyl group.

A substituent of “substituted alkyl group having 1 to 20 carbon atoms” may be an unsubstituted alkyl group having 1 to 20 carbon atoms. The term “substituted alkyl group having 1 to 20 carbon atoms” refers to a group formed by substituting an unsubstituted alkyl group having 1 to 20 carbon atoms with a substituent. Accordingly, the number of carbon atoms in the substituted alkyl group may be greater than 20.

In (a4), the alkoxy group having 1 to 20 carbon atoms is not particularly limited, and the alkoxy group may be linear, branched, or cyclic. In particular, the alkoxy group may be linear to improve device lifespan. The number of carbon atoms of the alkoxy group may be 1 or more and 10 or less to improve device lifespan. In the same point of view, the number of carbon atoms in the alkoxy group may be 1 to 8, for example, 1 to 6, or for example, 1. Examples of an alkyl group constituting the alkoxy group may include, but are not particularly limited to, those mentioned in the above description of the alkyl group. Examples of the alkoxy group may include, but are not particularly limited to, a methoxy group, an ethoxy group, an n-propoxy group, an isopropoxy group, a butoxy group, a pentyloxy group, a hexyloxy group, an octyloxy group, a nonyloxy group, a decyloxy group, and the like. In particular, the alkoxy group may be a methoxy group.

The term “substituted alkoxy group having 1 to 20 carbon atoms” refers to a group formed by substituting an unsubstituted alkoxy group having 1 to 20 carbon atoms with a substituent. Accordingly, the number of carbon atoms in the substituted alkoxy group may be greater than 20.

A nitrogen atom of the arylamino group having 6 to 20 carbon atoms of (a5) may be linked to ring-forming carbon atoms to which R² or R⁴ in Formula (1) may be linked via a single bond. Herein, even in a case where a group includes a nitrogen atom, when the nitrogen atom is a ring-forming atom of a hetero ring, the group is handled as a heterocyclic group to be described below, instead of an arylamino group. Examples of an aryl group constituting the arylamino group may include, but are not particularly limited to, a group having 6 to 20 carbon atoms among aromatic hydrocarbon groups of (a7) to be described below. The arylamino group may be, but is not particularly limited to, a monoarylamino group or a diarylamino group. Examples of the arylamino group may include, but are not particularly limited to, a N-phenylamino group, a N-biphenylamino group, a N-terphenylamino group, a N,N-diphenylamino group, a N-biphenyl-N-phenylamino group, and the like.

The term “substituted arylamino group having 6 to 20 carbon atoms” refers to a group formed by substituting an unsubstituted arylamino group having 6 to 20 carbon atoms with a substituent. Accordingly, the number of carbon atoms in the substituted arylamino group may be greater than 20.

Silicon (Si) of a triarylsilyl group, an alkyldiarylsilyl group, a dialkylarylsilyl group, or a trialkylsilyl group of (a6) may be bonded via a single bond to a ring-forming carbon atom to which R² or R⁴ in Formula (1) may be bonded. An aryl group constituting a triarylsilyl group, an alkyldiarylsilyl group, a dialkylarylsilyl group, or a trialkylsilyl group may be an aryl group having 6 to 20 carbon atoms, for example, a group having 6 to 20 carbon atoms from among aromatic hydrocarbon groups to be described below. An alkyl group constituting a triarylsilyl group, an alkyldiarylsilyl group, a dialkylarylsilyl group, or a trialkylsilyl group may be an alkyl group having 1 to 20 car on atoms, and the alkyl group having 1 to 20 carbon atoms described in connection with (a3) may be applicable. Examples of a triarylsilyl group are not particularly limited and may include a triphenylsilyl group, a tri (tert-butylphenyl) silyl group, a di-tert-butylphenyl(phenyl) silyl group, etc. Examples of an alkyldiarylsilyl group are not particularly limited and may include a diphenylmethylsilyl group, a diphenyl(tert-butyl) silyl group, a di-tert-butylphenyl(methyl) silyl group, a di-tert-butylphenyl(tert-butyl) silyl group, etc. Examples of a dialkylarylsilyl group are not particularly limited and may include a dimethylphenylsilyl group, etc. Examples of a trialkylsilyl group are not particularly limited and may include a trimethylsilyl group, a tri-tert-butylsilyl group, a di-tert-butyl(methyl) silyl group, etc.

In addition, the term “substituted triarylsilyl group, alkyldiarylsilyl group, dialkyl arylsilyl group, or trialkylsilyl group” refers to a group formed by substituting an unsubstituted aryl group having 6 to 20 carbon atoms and an alkyl group having 1 to 20 carbon atoms with a substituent. Accordingly, the number of carbon atoms of an aryl group of substituted triarylsilyl group, alkyldiarylsilyl group, and dialkyl arylsilyl group may be greater than 20, and the number of carbon atoms of an alkyl group of substituted alkyldiarylsilyl group, dialkylarylsilyl group, and trialkylsilyl group may be greater than 20.

An aromatic hydrocarbon group having 6 to 30 carbon atoms described in connection with (a7) refers to a group derived from at least one aromatic hydrocarbon ring. As used herein, the term “aromatic hydrocarbon ring” refers to a hydrocarbon ring that is aromatic.

When the aromatic hydrocarbon group includes two or more aromatic hydrocarbon rings, the rings may be bonded via a single bond or condensed to each other. In addition, when the aromatic hydrocarbon group includes two or more aromatic hydrocarbon rings, one atom may serve as a ring-forming atom of any of the rings.

To improve the luminescence color purity, the number of carbon atoms in the aromatic hydrocarbon group may be 6 to 20, for example, 6 to 12, or for example, 6.

Examples of the aromatic hydrocarbon group may include, but are not particularly limited to, a phenyl group, a mesityl group, a tert-butylphenyl group, a bis(tert-butyl)phenyl group, a biphenyl group, a terphenyl group, a naphthyl group, a fluorenyl group, an anthracenyl group, a terphenyl group, a quaterphenyl group, a quinquephenyl group, a sexiphenyl group, a triphenylenyl group, a pyrenyl group, a benzofluorenyl group, a chrysenyl group, and a combination thereof.

The term “substituted aromatic hydrocarbon group” refers to a group formed by substituting an unsubstituted aromatic hydrocarbon group with a substituent. Accordingly, when the number of carbon atoms in the aromatic hydrocarbon group is equal to or less than a certain upper limit of the number of carbon atoms, for example, 30 or less, the number of carbon atoms in the substituted aromatic hydrocarbon group may be greater than the upper limit.

A heterocyclic group having 3 to 30 ring-forming atoms of (a8) refers to a group derived from at least one hetero ring. The heterocyclic group may be, but is not particularly limited to, an aromatic heterocyclic group or a non-aromatic heterocyclic group. In particular, the heterocyclic group may be an aromatic heterocyclic group to improve luminescence color purity.

The term “aromatic heterocyclic group” refers to a group derived from at least one aromatic hetero ring. As used herein, the term “aromatic hetero ring” refers to a hetero ring that is aromatic. When the aromatic hetero ring is partially aromatic, aromaticity may be derived from a heterocyclic part of the ring or from a hydrocarbon ring part of the ring. The aromatic hetero ring is not particularly limited, and may be, for example, a ring having at least one hetero atom (e.g., a nitrogen atom (N), an oxygen atom (O), a phosphorus atom (P), a sulfur atom(S), or a silicon atom (Si)) as a ring-forming atom, wherein the remaining ring-forming atoms are carbon atoms (C). An atom constituting a ring structure may be bonded to an exocyclic atom via a double bond. For example, a carbon atom constituting the ring structure may constitute a ketone group (CO group), a thioketone group (C═S group), or a C═NH group, or a sulfur atom constituting the ring structure may constitute a sulfinyl group (S═O group) or a sulfonyl group (S(═O)═O group). In this case, as used herein, the exocyclic atom forming the double bond with the atom constituting the ring structure may be part of the aromatic hetero ring. In addition, when the exocyclic atom forming the double bond is bonded to a hydrogen atom via a single bond, the hydrogen atom may also be part of the aromatic hetero ring. Examples of the aromatic hetero ring may include, but are not particularly limited to, a pyridine ring, a pyrazine ring, a pyridazine ring, a pyrimidine ring, a triazine ring, a quinoline ring, an isoquinoline ring, a quinoxaline ring, a quinazoline ring, a naphthyridine ring, an acridine ring, a phenazine ring, a benzoquinoline ring, a benzoisoquinoline ring, a phenanthridine ring, a phenanthroline ring, a benzoquinone ring, a coumarin ring, an anthraquinone ring, a fluorenone ring, a furan ring, a thiophene ring, a benzofuran ring, a benzothiophene ring, a dibenzofuran ring, a dibenzothiophene ring, a pyrrole ring, an indole ring, a carbazole ring, an indolecarbazole ring, an imidazole ring, a benzimidazole ring, a pyrazole ring, an indazole ring, an oxazole ring, an isoxazole ring, a benzoxazole ring, a benzoisoxazole ring, a thiazole ring, an isothiazole ring, a benzothiazole ring, a benzoisothiazole ring, an imidazolinone ring, a benzimidazolinone ring, an imidazopyridine ring, an imidazopyrimidine ring, an imidazophenanthridine ring, a benzimidazophenanthridine ring, an azadibenzofuran ring, an azacarbazole ring, an azadibenzothiophene ring, a diazadibenzofuran ring, a diazacarbazole ring, a diazadibenzothiophene ring, a xanthone ring, a thioxanthone ring, and the like.

When the aromatic heterocyclic group includes two or more aromatic hetero rings, the rings may be bonded via a single bond or condensed to each other. In addition, when the aromatic heterocyclic group includes two or more aromatic hetero rings, one atom may serve as a ring-forming atom of any of the rings.

The number of ring-forming atoms in the aromatic heterocyclic group (the sum of the number of ring-forming carbon atoms and the number of ring-forming hetero atoms) may be 3 to 30, and may preferably be 5 to 20 or 6 to 14 to provide the desired peak wavelength of the emission spectrum and luminescence color purity. The number of ring-forming hetero atoms in the aromatic heterocyclic group may be, but is not particularly limited to, 1 to 10, to provide the desired peak wavelength of the emission spectrum and luminescence color purity. In the same point of view, the number of ring-forming hetero atoms in the aromatic heterocyclic group may be 1 to 5, for example, 1 to 3. As described above, the term “ring-forming atom” refers to an atom that directly forms a ring structure.

Examples of the aromatic heterocyclic group may include, but are not particularly limited to, a thienyl group, a furanyl group, a pyrrolyl group, an imidazolyl group, a thiazolyl group, an oxazolyl group, an oxadiazolyl group, a triazolyl group, a pyridyl group, a bipyridyl group, a pyrimidyl group, a triazinyl group, an acridinyl group, a pyridazinyl group, a pyrazinyl group, a quinolinyl group, a quinazolinyl group, a quinoxalinyl group, a phenoxazinyl group, a phthalazinyl group, a pyridpyrimidinyl group, a pyridpyrazinyl group, a pyrazinopyrazinyl group, an isoquinolinyl group, an indolyl group, a carbazolyl group, a benzoxazolyl group, a benzimidazolyl group, a benzothiazolyl group, a benzocarbazolyl group, a benzothiophenyl group, a dibenzothiophenyl group, a thienothienyl group, a benzofuranyl group, a phenanthrolinyl group, a thiazolyl group, an isoxazolyl group, an oxadiazolyl group, a thiadiazolyl group, a phenothiazinyl group, a dibenzosilolyl group, a dibenzofuranyl group, a xanthonyl group, and the like. In an embodiment, the aromatic heterocyclic group may preferably be a triazinyl group, a carbazolyl group, a benzoxazolyl group, or a xanthonyl group.

In addition, the term “non-aromatic heterocyclic group” refers to a group derived from at least one non-aromatic hetero ring. As used herein, the term “non-aromatic hetero ring” refers to a hetero ring that is entirely non-aromatic. The non-aromatic hetero ring is not particularly limited, and may be, for example, a ring having at least one hetero atom (e.g., a nitrogen atom (N), an oxygen atom (O), a phosphorus atom (P), a sulfur atom(S), or a silicon atom (Si)) as a ring-forming atom, wherein the remaining ring-forming atoms are carbon atoms (C). The hetero atom may be a nitrogen atom (N) or an oxygen atom (O), to provide the desired peak wavelength of the emission spectrum and luminescence color purity. An atom constituting a ring structure may be bonded to an exocyclic atom via a double bond. For example, a carbon atom constituting the ring structure may constitute a ketone group (CO group), a thioketone group (C═S group), or a C═NH group, or a sulfur atom constituting the ring structure may constitute a sulfinyl group (S═O group) or a sulfonyl group (S(═O)═O group). In this case, as used herein, the exocyclic atom forming the double bond with the atom constituting the ring structure may be part of the non-aromatic hetero ring. In addition, when the exocyclic atom forming the double bond is bonded to a hydrogen atom via a single bond, the hydrogen atom may also be part of the non-aromatic hetero ring. Examples of the non-aromatic hetero ring may include, but are not particularly limited to, a pyrrolidine ring, tetrahydrofuran ring, a tetrahydrothiophene ring, a piperidine ring, a tetrahydropyran ring, a tetrahydrothiopyran ring, a dioxane ring, a morpholine ring, a dioxolane ring, and the like.

When the non-aromatic heterocyclic group includes two or more non-aromatic hetero rings, the rings may be bonded via a single bond or condensed to each other. In addition, when the non-aromatic heterocyclic group includes two or more non-aromatic hetero rings, one atom may serve as a ring-forming atom of any of the rings.

The number of ring-forming atoms in the non-aromatic heterocyclic group (the sum of the number of ring-forming carbon atoms and the number of ring-forming hetero atoms) may be 3 to 30, and may preferably be 5 to 20 or 6 to 14 to provide the desired peak wavelength of the emission spectrum and luminescence color purity. The number of ring-forming hetero atoms in the non-aromatic heterocyclic group may be, but is not particularly limited to, 1 to 10, to provide the desired peak wavelength of the emission spectrum and luminescence color purity. In the same point of view, the number of ring-forming hetero atoms in the non-aromatic heterocyclic group may be 1 to 5, for example, 1 to 3. As described above, the term “ring-forming atom” refers to an atom that directly forms a ring structure. In this regard, when there is an exocyclic atom forming a double bond with an atom constituting a ring structure, the exocyclic atom is not a ring-forming atom.

Examples of the non-aromatic heterocyclic group may include, but are not particularly limited to, a pyrrolidinyl group, a tetrahydrofuranyl group, a tetrahydrothienyl group, a piperidinyl group, a tetrahydropyranyl group, a tetrahydrothiopyranyl group, a dioxanyl group, a morphonyl group, a dioxolanyl group, and the like.

The term “substituted heterocyclic group” refers to a group formed by substituting an unsubstituted heterocyclic group with a substituent. Accordingly, when the number of ring-forming atoms in the heterocyclic group is equal to or less than a certain upper limit of the number of ring-forming atoms, for example, 30 or less, the number of ring-forming atoms in the substituted heterocyclic group may be greater than the upper limit.

A substituted or unsubstituted saturated hydrocarbon group or saturated heterocyclic group having 5 to 9 ring-forming atoms and formed by two R² bonded to each other when n1 is 2 or more and a substituted or unsubstituted saturated hydrocarbon group or saturated heterocyclic group having 5 to 9 ring-forming atoms and formed by two R⁴ bonded to each other when n2 is 2 or more as described in (a9) may be bonded via a single bond to a ring-forming carbon atom to which two R² or R⁴ may be bonded. That is, a saturated hydrocarbon group or saturated heterocyclic group having 5 to 9 ring-forming atoms linked to two positions refers to a saturated hydrocarbon cyclic group or saturated heterocyclic group condensed with a ring constituted by ring-forming carbon atoms and formed by substituting ring-forming carbon atoms, to which R² or R⁴ may be bonded, with two R² or R⁴. The substitution positions of R² and R⁴ are not particularly limited, and may be a condensed ring with a bridge structure in which two carbon atoms which are not adjacent to each other in a ring constituted by ring-forming carbon atoms are substituted or may preferably be a condensed ring in which two carbon atoms which are adjacent to each other in a ring constituted by ring-forming atoms are constituted.

Examples of the condensed ring (saturated hydrocarbon ring) formed by a saturated hydrocarbon group having 5 to 9 ring-forming atoms may include a cyclopentane ring, a cyclohexane ring, a cycloheptane ring, a cyclooctane ring, a cyclononane ring, etc. Examples of the condensed ring (saturated hetero ring) formed by a saturated heterocyclic group having 5 to 9 ring-forming atoms may include a group having 5 to 9 ring-forming atoms, etc. from among the non-aromatic heterocyclic groups. Specific examples of the condensed ring (saturated hetero ring) formed by a saturated heterocyclic group having 5 to 9 ring-forming atoms may include a pyrrolidine ring, a tetrahydrofuran ring, a tetrahydrothiophene ring, a piperidine ring, a tetrahydropyran ring, a tetrahydrothiopyran ring, a dioxane ring, a morpholine ring, a dioxolan ring, etc.

In addition, the term “substituted saturated hydrocarbon group or saturated heterocyclic group having 5 to 9 ring-forming atoms” refers to a group formed by substituting an unsubstituted saturated hydrocarbon group or saturated heterocyclic group with a substituent. Accordingly, when the number of ring-forming atoms in the saturated hydrocarbon group or saturated heterocyclic group is equal to or less than a certain upper limit of the number of ring-forming atoms, for example, 9 or less, the number of ring-forming atoms in the substituted saturated hydrocarbon group or saturated heterocyclic group may be greater than the upper limit.

A halogen atom, a cyano group, a substituted or unsubstituted alkyl group having 1 to 20 carbon atoms, an unsubstituted alkoxy group having 1 to 20 carbon atoms, an unsubstituted arylamino group having 6 to 20 carbon atoms, a substituted or unsubstituted aromatic hydrocarbon group having 6 to 30 carbon atoms, and an unsubstituted heterocyclic group having 3 to 30 ring-forming atoms, which are a substituent substituting the groups of (a1) to (a9), may be the same as an unsubstituted group described in connection with (a3) to (a9).

In an unsubstituted alkylamino group having 1 to 20 carbon atoms, which is a substituent substituting the groups of (a3) to (a9), the nitrogen atoms may bond with any one of atoms constituting the unsubstituted groups of (a3) to (a9) of Formula (1) via a single bond. The alkyl group constituting the alkylamino group is not particularly limited, but may be, for example, the same as described in connection with (a3). The alkylamino group may be, but is not particularly limited to, a monoalkylamino group or a dialkylamino group. Examples of the alkylamino group may include, but particularly limited to, an N-methylamino group, an N-ethylamino group, an N-propylamino group, an N-isopropylamino group, an N-butylamino group, an N-isobutylamino group, an N-sec-butylamino group, an N-tert-butylamino group, an N-pentylamino group, an N-hexylamino group, an N,N, N-dimethylamino group, an N-methyl-N-ethylamino group, an N,N-diethylamino group, an N,N-dipropylamino group, an N,N-diisopropylamino group, an N,N-dibutylamino group, an N,N-diisobutylamino group, an N,N-dipentylamino group, and an N, N-dihexylamino group.

An unsubstituted halo alkyl group having 1 to 20 carbon atoms, which is a substituent substituting the groups of (a3) to (a9) may be a group wherein at least one hydrogen atom of the alkyl group described in (a3) is substituted with the halogen atom described in (a1). The halogen atom may be a fluorine atom to improve device lifespan. Examples of the alkyl group may include a trifluoromethyl group, a trichloromethyl group, a tribromomethyl group, a triiodomethyl group, and the like. In an embodiment, the alkyl group may be a fluoro alkyl group and a trifluoromethyl group.

A preferred substituent substituting the groups of (a3) to (a9) may be a halogen atom, a cyano group, an unsubstituted alkyl group having 1 to 20 carbon atoms, an unsubstituted alkoxy group having 1 to 20 carbon atoms, or an unsubstituted arylamino group having 6 to 20 carbon atoms. Moreover, from among the above, the substituent may be a halogen atom or an unsubstituted alkyl group having 1 to 20 carbon atoms, or may preferably be a fluorine atom or an unsubstituted linear or branched alkyl group having 1 to 20 carbon atoms. The substituent may be, for example, a fluorine atom, a methyl group, an ethyl group, an iso-propyl group, or a tert-butyl group.

The substituent substituting the group of (a7) may be an unsubstituted alkyl group having 1 to 20 carbon atoms. From among the above, the substituent may be an unsubstituted linear or branched alkyl group having 1 to 20 carbon atoms. The substituent may be, for example, a methyl group, an ethyl group, an iso-propyl group, or a t-butyl group.

The substituent substituting the group of (a7) may be an unsubstituted heterocyclic group having 3 to 30 ring-forming atoms. In an embodiment, the heteroatom may be a heterocyclic group including oxygen or nitrogen atoms, for example, a dibenzofuranyl group, a carbazolyl group, or a benzoxazolyl group. The heteroatom may preferably be a dibenzofuranyl group or a carbazolyl group.

The substituent substituting the groups of (a3), (a4), and (a8) may be an unsubstituted aromatic hydrocarbon group having 6 to 30 carbon atoms. The unsubstituted aromatic hydrocarbon group may be a group derived from a benzene ring.

When the groups of (a3) to (a9) are a substituted group, the substituent may be a group substituted with an additional substituent. The additional substituent may include, but is not particularly limited to, a substituent of the case where the groups of (a3) to (a9) are substituted groups or a group additionally substituted by the aforementioned groups.

Of the substituted aromatic hydrocarbon group having 6 to 30 carbon atoms substituting the groups of (a3) to (a9), an aromatic hydrocarbon group having 6 to 30 carbon atoms is the same as the unsubstituted group described in connection with (a7) except for a limited range of the number of carbon atoms. The substituted aromatic hydrocarbon group may be a group derived from a benzene ring. In addition, a substituent substituting an aromatic hydrocarbon group having 6 to 30 carbon atoms is not particularly limited, but may include an unsubstituted alkyl group having 1 to 20 carbon atoms. The unsubstituted alkyl group having 1 to 20 carbon atoms is the same as the unsubstituted group described in connection with (a3). From among the above, the unsubstituted alkyl group may be an unsubstituted linear or branched alkyl group having 1 to 20 carbon atoms. The unsubstituted alkyl group may be, for example, a methyl group, an ethyl group, an iso-propyl group, or a tert-butyl group.

Of the substituted alkyl group having 1 to 20 carbon atoms substituting the groups of (a3) to (a9), an alkyl group having 1 to 20 carbon atoms is the same as the unsubstituted group described in connection with (a3). From among the above, the substituted alkyl group may be a linear or branched alkyl group having 1 to 20 carbon atoms. The substituted alkyl group may be, for example, a methyl group, an ethyl group, an iso-propyl group, or a tert-butyl group. In addition, a substituent substituting an alkyl group having 1 to 20 carbon atoms is not particularly limited, but may include an aromatic hydrocarbon group having 6 to 30 carbon atoms and substituted or unsubstituted with an unsubstituted alkyl group having 1 to 20 carbon atoms. In this regard, the unsubstituted alkyl group having 1 to 20 carbon atoms that substitutes the aromatic hydrocarbon group having 6 to 30 carbon atoms is the same as the unsubstituted group described in connection with (a3). From among the above, the unsubstituted alkyl group may be an unsubstituted linear or branched alkyl group having 1 to 20 carbon atoms. The unsubstituted alkyl group may be, for example, a methyl group, an ethyl group, an iso-propyl group, or a tert-butyl group.

R¹ and R³ in the compound represented by Formula (1) may have a structure represented by Formula (2):

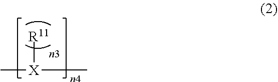

R¹¹ may each independently be any one atom or group selected from (b1) to (b7):
(b1) a hydrogen atom or a deuterium atom;
(b2) a cyano group;
(b3) a substituted or unsubstituted alkyl group having 1 to 20 carbon atoms;
(b4) a substituted or unsubstituted alkoxy group having 1 to 20 carbon atoms;
(b5) a substituted or unsubstituted arylamino group having 6 to 20 carbon atoms;
(b6) a substituted or unsubstituted aromatic hydrocarbon group having 6 to 30 carbon atoms; and
(b7) a substituted or unsubstituted heterocyclic group having 6 to 30 ring-forming atoms,
wherein at least one of R¹¹ may be selected from (b2) to (b7),
wherein at least one of X is a carbon atom, X are each independently a carbon atom, a silicon atom, a nitrogen atom, an oxygen atom, a sulfur atom, or a selenium atom, and a plurality of X are identical to or different from each other, and
n3 may each independently be 0, 1, or 2, and when n3 is 2, R¹¹ may be identical to or different from each other, and
n4 may be 3, 4, 5, 6, or 7.

As described above, n3 in Formula (2) may be 0, 1, or 2 based on the valence of X, and when n3 is 2, R¹¹ may be identical to or different from each other. When R¹ and R³ have a structure represented by Formula (2), from among a plurality of X in Formula (2) (3 to 7), two X at either end may be bonded via a single bond to a ring-forming atom to which R¹ or R³ may be bonded. That is, the structure represented by Formula (2) refers to a ring condensed with a ring to which R¹ or R³ may be bonded.

A condensed ring formed by the structure represented by Formula (2) may be a substituted or unsubstituted saturated hydrocarbon cyclic group or saturated heterocyclic group. A saturated hydrocarbon group and saturated heterocyclic group unsubstituted with the structure represented by Formula (2) may be identical to, for example, the condensed ring formed by unsubstituted saturated hydrocarbon cyclic group and saturated heterocyclic group described in connection with (a9). Specific examples thereof may include a cyclopentane ring, a cyclohexane ring, a cycloheptane ring, a cyclooctane ring, a cyclononane ring, a pyrrolidine ring, a tetrahydrofuran ring, a tetrahydrothiophene ring, a piperidine ring, a tetrahydropyran ring, a tetrahydrothiopyran ring, a dioxane ring, a morpholine ring, a dioxolan ring, etc.

The term “substituted saturated hydrocarbon cyclic group or saturated heterocyclic group” refers to a group formed by substituting an unsubstituted saturated hydrocarbon cyclic group or saturated heterocyclic group with a substituent. Accordingly, when the number of ring-forming atoms in the saturated hydrocarbon cyclic group or saturated heterocyclic group is equal to or less than a certain upper limit of the number of ring-forming atoms, for example, 9 or less, the number of ring-forming atoms in the substituted saturated hydrocarbon cyclic group or saturated heterocyclic group may be greater than the upper limit.

A halogen atom, an unsubstituted alkyl group having 1 to 20 carbon atoms, an unsubstituted alkoxy group having 1 to 20 carbon atoms, an unsubstituted arylamino group having 6 to 20 carbon atoms, an unsubstituted aromatic hydrocarbon group having 6 to 30 carbon atoms, and an unsubstituted heterocyclic group having 6 to 20 ring-forming atoms described in (b3) to (b7) may each be identical to the unsubstituted group described in connection with (a1), (a3), (a4), (a5), (a7), and (a8).

A substituted alkyl group having 1 to 20 to carbon atoms, a substituted alkoxy group having 1 to 20 carbon atoms, a substituted arylamino group having 6 to 20 carbon atoms, a substituted aromatic hydrocarbon group having 6 to 30 carbon atoms, and a substituted heterocyclic group having 6 to 30 ring-forming atoms described in (b3) to (b7) may each be identical to the substituents substituting the groups of (a3) to (a9).

Here, R¹¹ may be a halogen atom or an unsubstituted alkyl group having 1 to 20 carbon atoms to provide improved luminescence color purity, luminescence efficiency, and device lifespan. R¹¹ may preferably be an unsubstituted alkyl group having 1 to 20 carbon atoms, for example, an unsubstituted linear or branched alkyl group having 1 to 20 carbon atoms. R¹¹ may be a methyl group, an iso-propyl group, or a tert-butyl group.

The compound represented by Formula (1) may have a structure represented by Formula (1A) or (1B) to provide the desired peak wavelength of emission spectrum and luminescence color purity. That is, a preferred embodiment of the disclosure relates to a compound having the structure represented by Formula (1A) or (1B). In an embodiment of a material for an organic EL device to be described below, the compound represented by Formula (1) may have a structure represented by Formula (1A) or (1B).

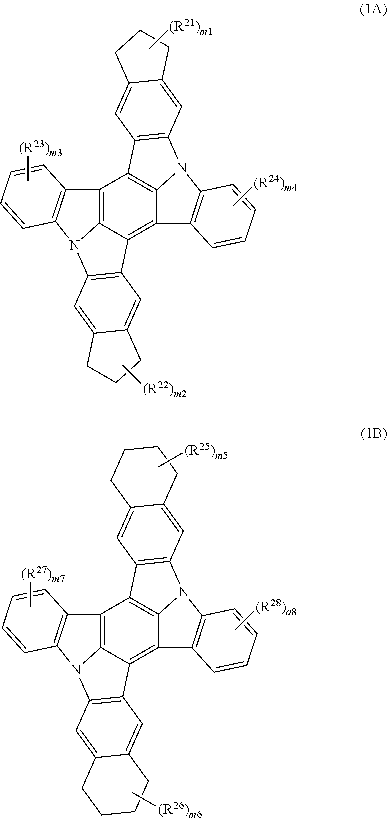

In Formulae (1A) and (1B),
R²¹, R²², R²⁵, and R²⁶ may each independently be any one atom or group selected from (c1) to (c6):
(c1) a cyano group;
(c2) a substituted or unsubstituted alkyl group having 1 to 20 carbon atoms;
(c3) a substituted or unsubstituted alkoxy group having 1 to 20 carbon atoms;
(c4) a substituted or unsubstituted arylamino group having 6 to 20 carbon atoms;
(c5) a substituted or unsubstituted aromatic hydrocarbon group having 6 to 30 carbon atoms; and
(c6) a substituted or unsubstituted heterocyclic group having 6 to 30 ring-forming atoms,
R²³, R²⁴, R²⁷, and R²⁸ may each independently be any one atom or group selected from (d1) to (d9):
(d1) a halogen atom;
(d2) a cyano group;
(d3) a substituted or unsubstituted alkyl group having 1 to 20 carbon atoms;
(d4) a substituted or unsubstituted alkoxy group having 1 to 20 carbon atoms;
(d5) a substituted or unsubstituted arylamino group having 6 to 20 carbon atoms;
(d6) a substituted or unsubstituted triarylsilyl group, a substituted or unsubstituted alkyldiarylsilyl group, a substituted or unsubstituted dialkylarylsilyl group, or a substituted or unsubstituted trialkylsilyl group, wherein an aryl group has 6 to 20 carbon atoms, and an alkyl group has 1 to 20 carbon atoms;
(d7) a substituted or unsubstituted aromatic hydrocarbon group having 6 to 30 carbon atoms;
(d8) a substituted or unsubstituted heterocyclic group having 3 to 30 ring-forming atoms; and
(d9) a substituted or unsubstituted saturated hydrocarbon group or saturated heterocyclic group having 5 to 9 ring-forming atoms and formed by two R²³ bonded to each other when m3 is 2, a substituted or unsubstituted saturated hydrocarbon group or saturated heterocyclic group having 5 to 9 ring-forming atoms and formed by two R²⁴ bonded to each other when m4 is 2, a substituted or unsubstituted saturated hydrocarbon group or saturated heterocyclic group having 5 to 9 ring-forming atoms and formed by two R²⁷ bonded to each other when m7 is 2, and a substituted or unsubstituted saturated hydrocarbon group or saturated heterocyclic group having 5 to 9 ring-forming atoms and formed by two R²⁸ bonded to each other when m8 is 2, and
wherein m1 and m2 may each independently be 0, 1, 2, 3, 4, 5 or 6, and when m1 is 2 or more, R²¹ may be identical to or different from each other, and when m2 is 2 or more, R²² may be identical to or different from each other,
m5 and m6 may each independently be 0, 1, 2, 3, or 4, and when m5 is 2 or more, R²⁵ may be identical to or different from each other, and when m6 is 2 or more, R²⁶ may be identical to or different from each other, and
m3, m4, m7, and m8 may each independently be 0, 1, 2, 3, or 4, and when m3 is 2 or more, R²³ may be identical to or different from each other, when m4 is 2 or more, R²⁴ may be identical to or different from each other, when m7 is 2 or more, R²⁷ may be identical to or different from each other, and when m8 is 2 or more, R²⁸ may be identical to or different from each other.

The groups of Formulae (1A) and (1B) are the same as described in relation to the groups of Formulae (1) and (2).

In the compound represented by Formula (1), the structure represented by Formula (1A) or (1B) may be a structure represented by any one of Formulae (1A-1), (1A-2), and (1B-1):

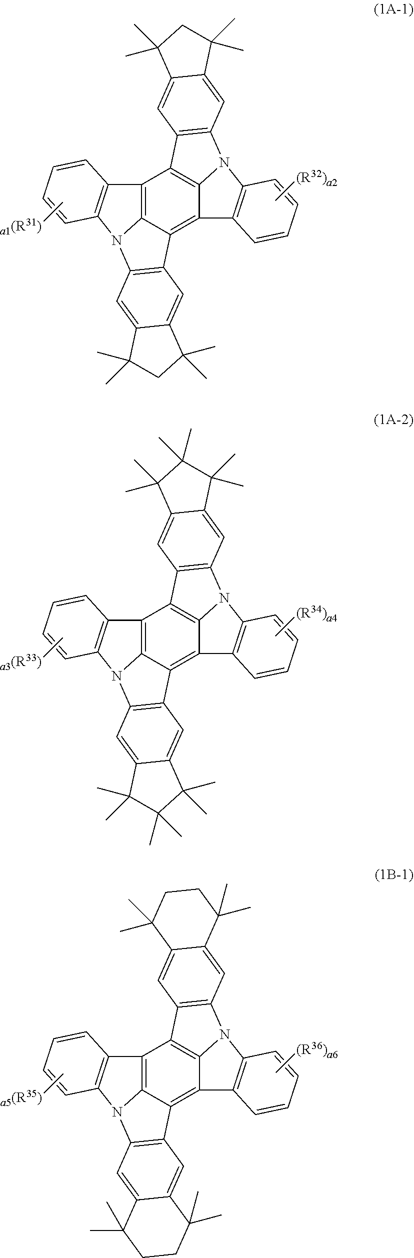

wherein, in Formulae (1A-1), (1A-2), and (1B-1),
R³¹ to R³⁶ may each independently be any one atom or group selected from (d1) to (d9),
a1 to a6 may each independently be 0, 1, 2, 3, or 4, and when a1 is 2 or more, R³¹ may be identical to or different from each other, when a2 is 2 or more, R³² may be identical to or different from each other, when a3 is 2 or more, R³³ may be identical to or different from each other, when a4 is 2 or more, R³⁴ may be identical to or different from each other, when a5 is 2 or more, R³⁵ may be identical to or different from each other, and when a6 is 2 or more, R³⁶ may be identical to or different from each other.

In the compounds represented by Formulae (1A), (1B), (1A-1), (1A-2), and (1B-1), R²³, R²⁴, R²⁷, R²⁸, and R³¹ to R³⁶ may be any one group selected from Group (X). In addition, “*” in the structural formula of Group (X) indicates a binding site to a benzene ring. When there are two “*” in the structural formula of Group (X), this indicates that benzene rings substituted with R²³, R²⁴, R²⁷, R²⁸, and R³¹ to R³⁶ are condensed.

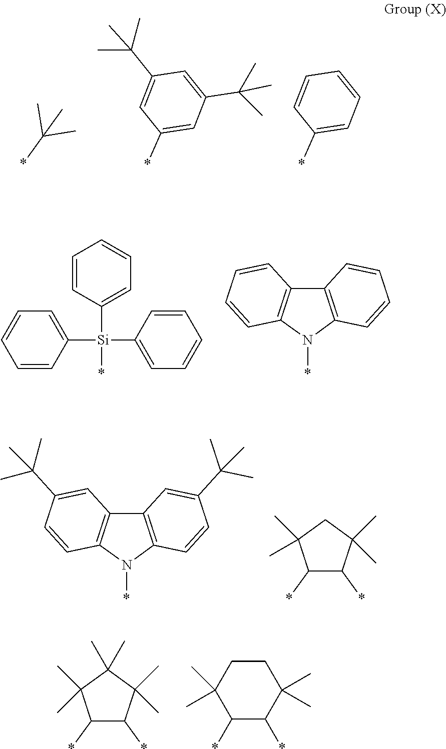

The substitution number in the benzene rings substituted with Group (X) (i.e., R²³, R²⁴, R²⁷, R²⁸, and R³¹ to R³⁶) may be 1 to 3 in each benzene ring, for example, 1 or 2. In addition, the substitution position of the benzene ring substituted with Group (X) is not particularly limited. For example, only the first (fourth) position may be substituted, only the second (third) position may be substituted, the first and the fourth positions may be substituted, or the second and the third positions may be substituted.

Hereinafter, the compound of Formula (1) according to an embodiment will be described in detail. However, the disclosure is not limited to such examples.

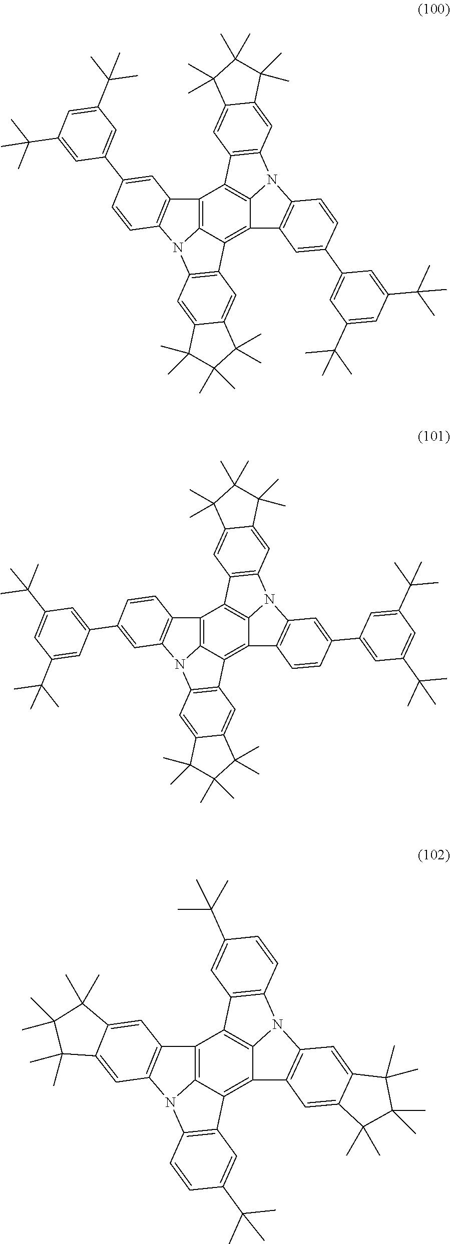

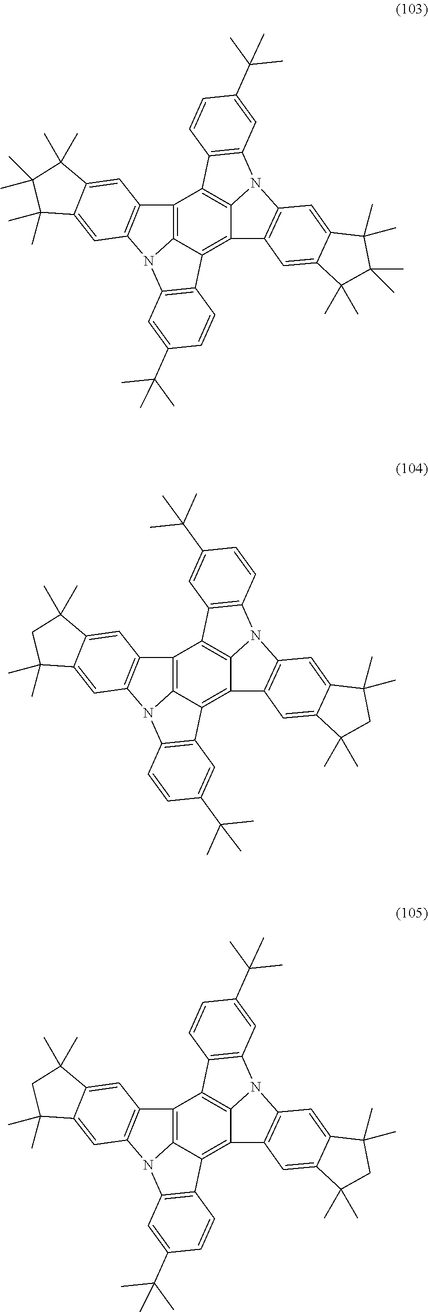

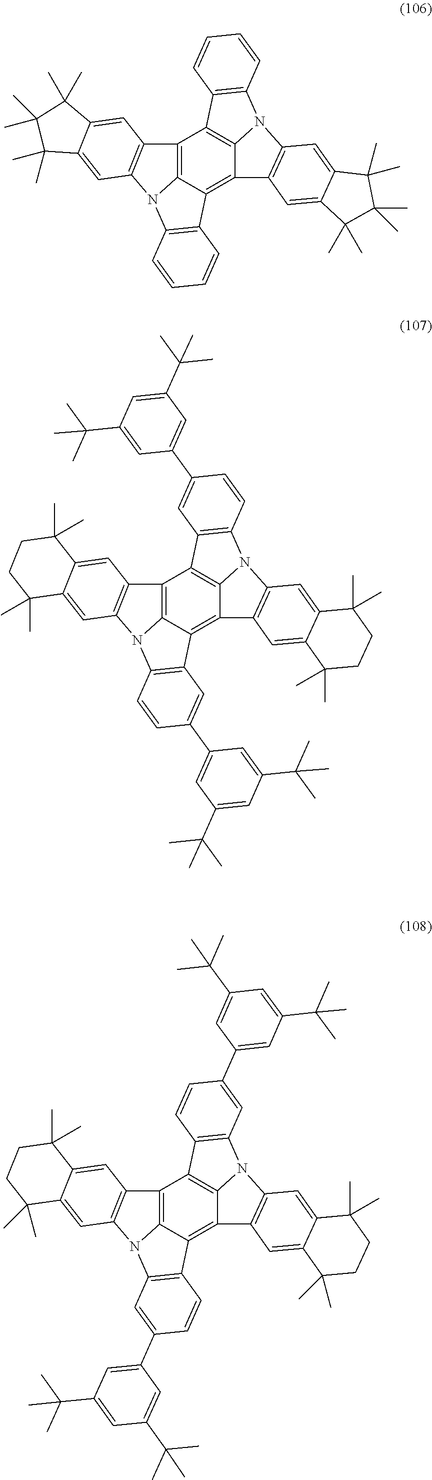

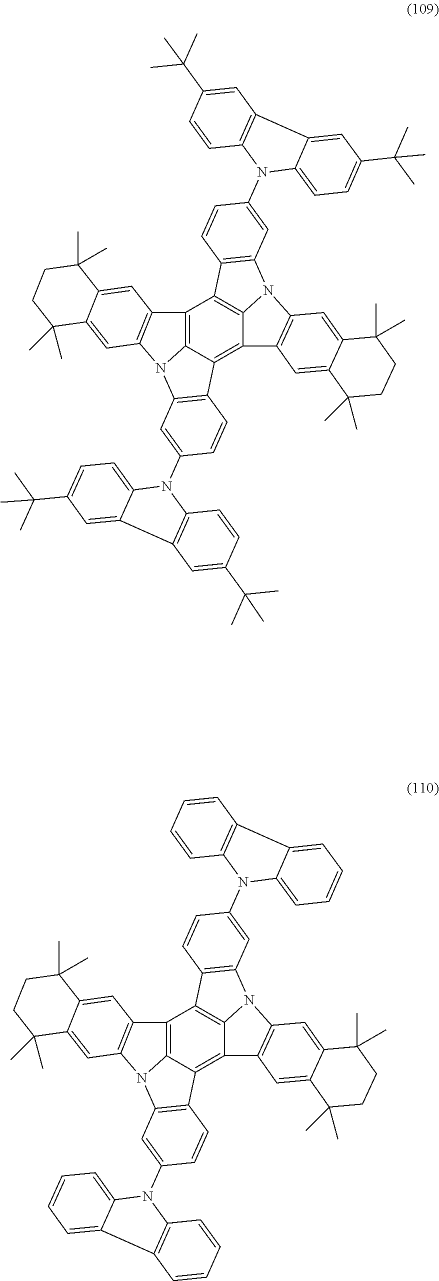

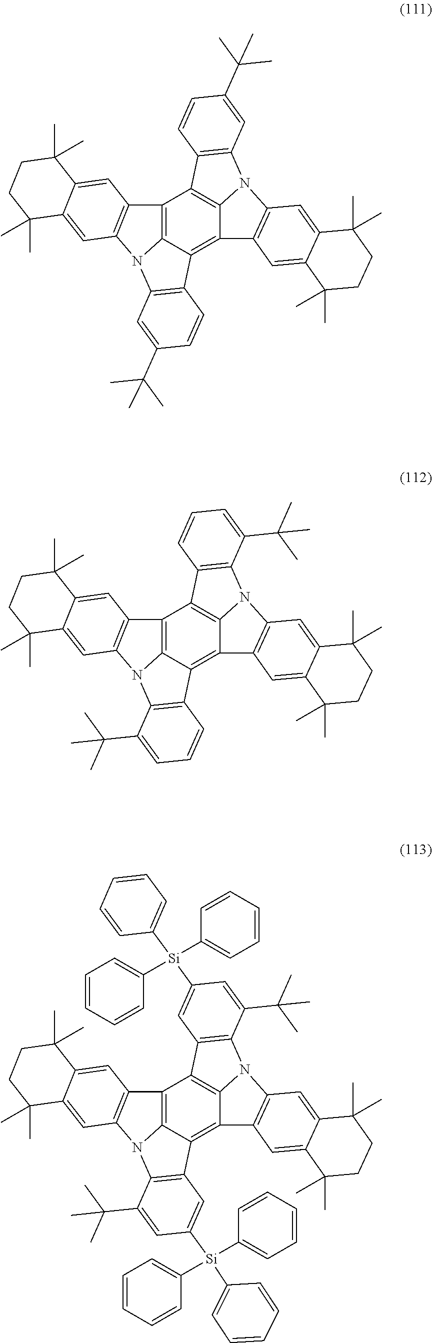

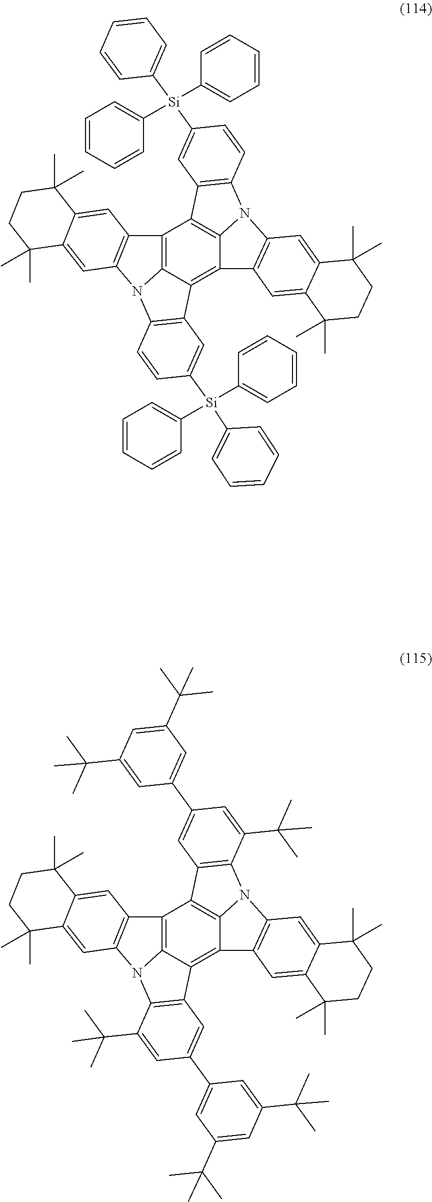

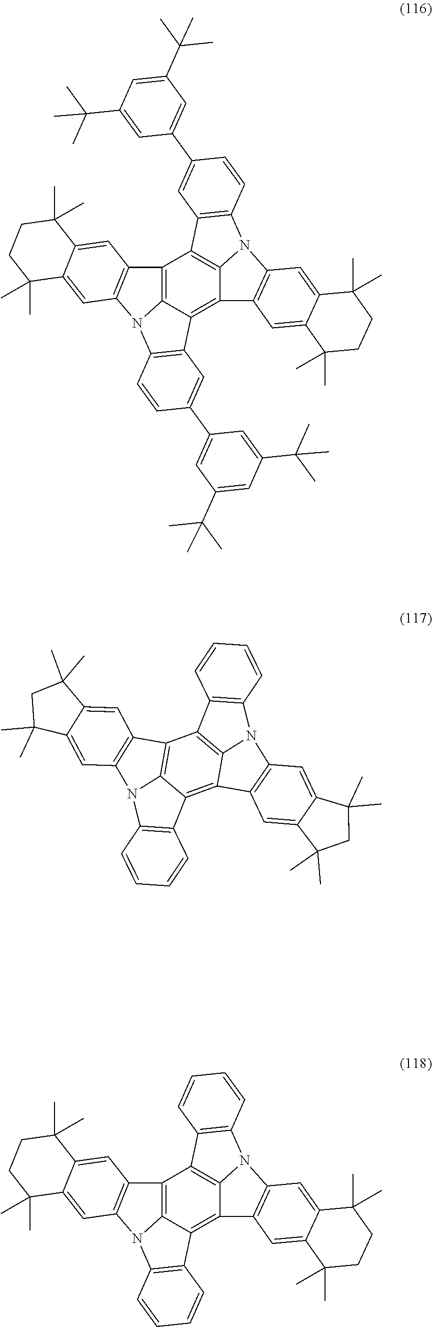

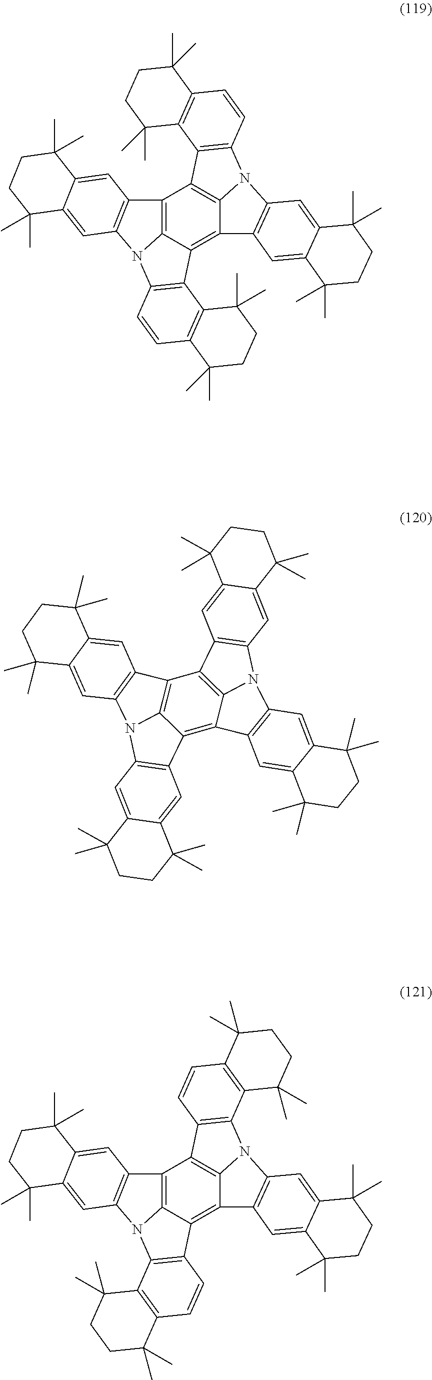

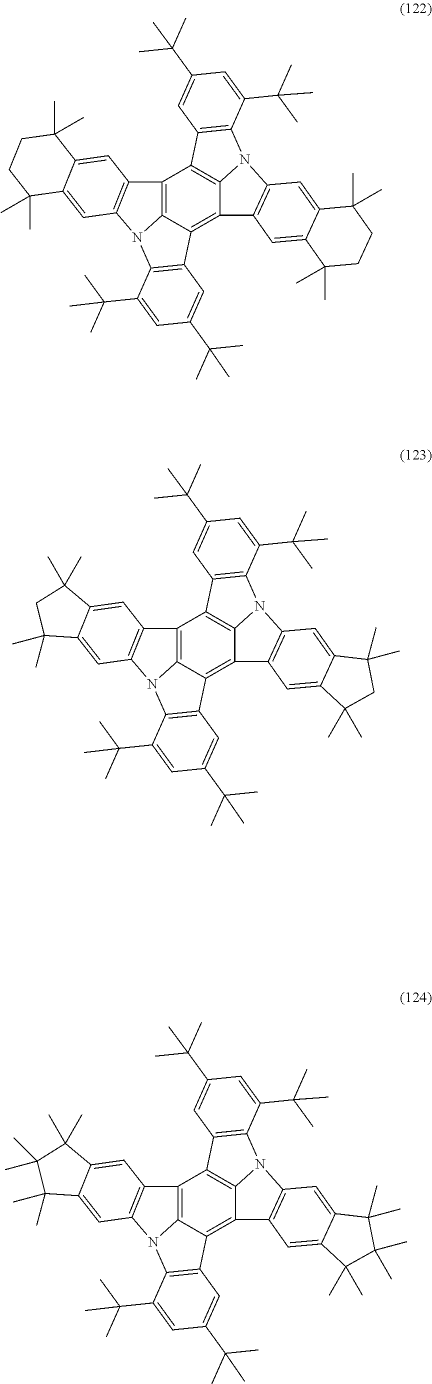

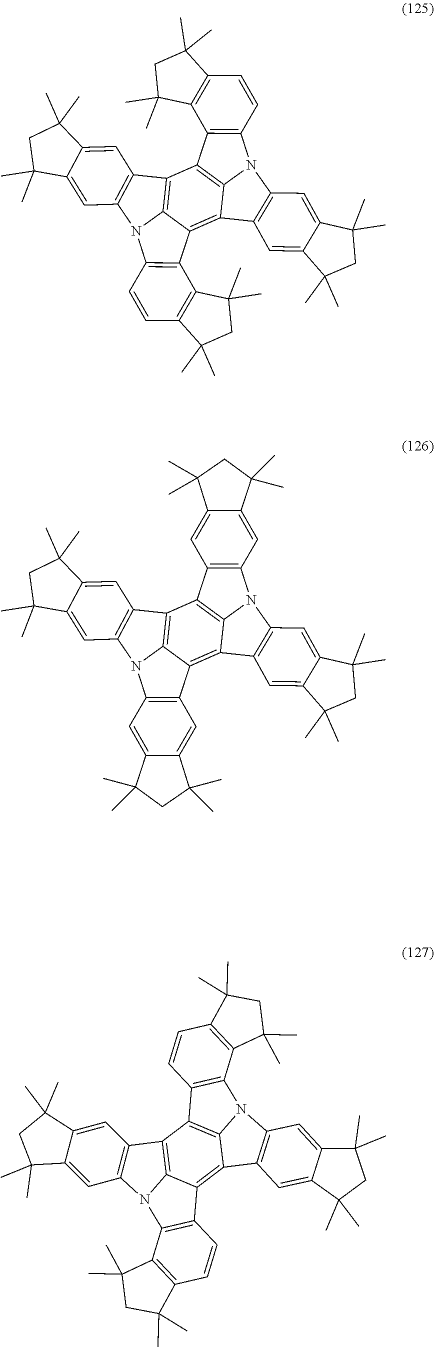

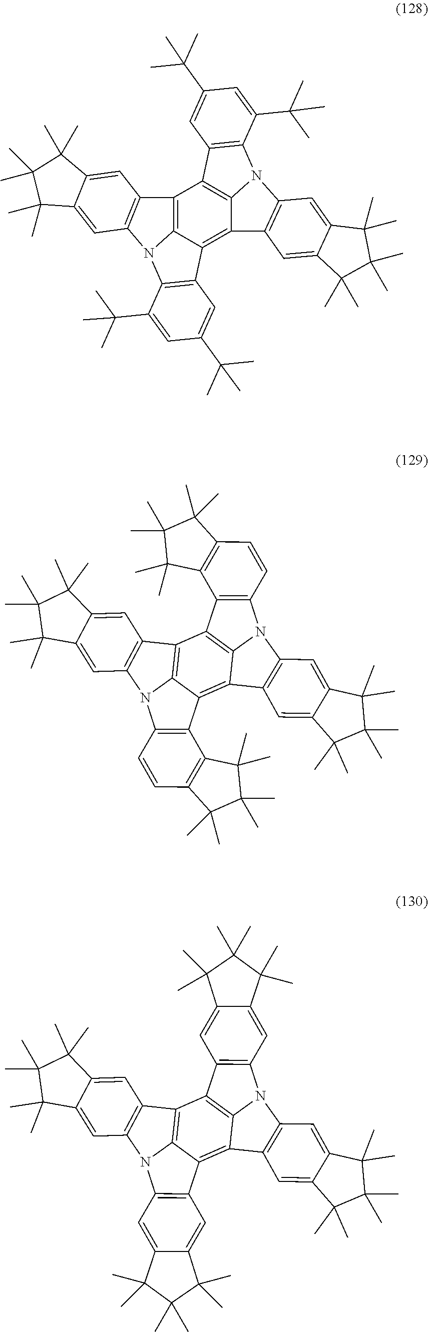

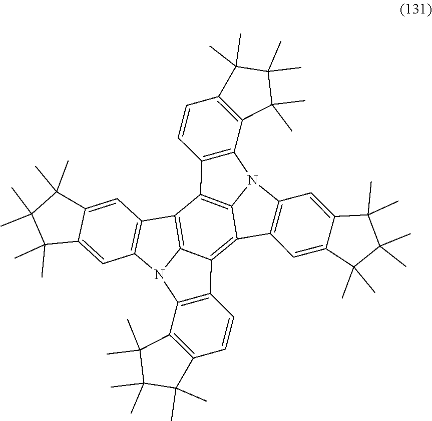

Preferred examples of the compound may include Compounds 101, 102, 108, 120, 126, and so forth.

The compound of Formula (1) according to the disclosure may have a peak wavelength of an emission spectrum in the blue wavelength region, and may provide luminescence with high color purity. The blue wavelength region as used herein refers to a wavelength region within a range of 380 nanometers (nm) or more and 500 nm or less. The photoluminescence (PL) peak wavelength of the compound of Formula (1) according to the disclosure may be, but not limited to, a range of 440 nm or more and 480 nm or less. The peak wavelength may emit light with a peak in a wavelength region of 445 nm to 470 nm, for example, a wavelength region of 450 nm to 470 nm or 450 nm to 465 nm. When the peak wavelength is within the above range, excellent luminescence, particularly, excellent blue luminescence may be obtained. The full width at half maximum (FWHM) of the peak of the emission spectrum of the photoluminescence (PL) may be 30 nm or less, 20 nm or less, and 15 nm or less (the lower limit exceeds 0 nm). The PL peak wavelength and the FWHM of the peak of the emission spectrum of the PL may be measured using a spectrofluorophotometer F-7000 of Hitachi Hightech Inc. More specifically, a thin film formed by depositing a 1×10⁻⁵ M (moles per cubic decimeter or moles per liter) of the compound of Formula (1) in a toluene solution, the compound of Formula (1), and host molecules according to the method described in the Example below were evaluated by measuring with the spectrofluorophotometer at room temperature with an excitation wavelength of 320 nm.

Narrow FWHM, thermally activated delayed fluorescence (TADF) characteristics, and emission wavelengths that are required for molecules used as dopants may be predicted by quantum chemistry calculations.

A method of synthesizing the compound of Formula (1) according to the disclosure is not particularly limited, and the compound may be synthesized by synthetic methods known in the art. In particular, the compound may be synthesized according to or in view of a method described in the Examples herein. For example, the compound may be synthesized by changing raw materials or reaction conditions in the method described in the Examples, adding or excluding some processes to or from the method described in the Examples, or appropriately combining the method described in the Examples with a known synthesis method.

A method of identifying the structure of the compound of Formula (1) according to the disclosure is not particularly limited. The structure of the compound according to the disclosure may be identified by known techniques (e.g., nuclear magnetic resonance spectroscopy, liquid chromatography mass spectroscopy, and so forth

### Material for Organic EL Device

Another aspect of the disclosure relates to a material for an organic EL device which includes the compound of Formula (1). The material may be a material for an emission layer.

The material for an organic EL device according to an embodiment may include the compound of Formula (1) and other materials used in an organic EL device. The other materials used in an organic EL device may be, but are not particularly limited to, phosphorescent compounds or host materials. In addition, the other materials may be a phosphorescent complex and a host material. In this regard, the compound of Formula (1) may be used as a dopant material, and the phosphorescent complex may be used as an auxiliary dopant. By using the compound of Formula (1) with the phosphorescent complex or the host material (or with the phosphorescent complex and the host material), luminescence efficiency and device lifespan may be significantly improved. While not wishing to be bound by theory, the reason is assumed as described below. When the material for an organic EL device contains the host material, the phosphorescent complex receives energy from the host material. In addition, the phosphorescent complex transfers energy to the compound of Formula (1) by a fluorescence resonance energy transfer (FRET) mechanism. As a result, energy may be transferred with high efficiency from the phosphorescent complex to the compound of Formula (1).

In addition, the other materials used in an organic EL device may include materials known in the art.

In addition, an amount of the compound of Formula (1) may be, but is not particularly limited to, 0.05 weight percent (wt %) or more based on the total weight of the material for an organic EL device (in particular, the material for an emission layer). In addition, the amount may be 0.1 wt % or more, for example, 0.2 wt % or more. When the amount is within the ranges above, an organic EL device with excellent luminescence color purity and high luminescence efficiency may be obtained. In addition, an amount of the compound of Formula (1) may be, but is not particularly limited to, 50 wt % or less based on the total weight of the material for an organic EL device (in particular, the material for an emission layer). In addition, the amount may be 30 wt % or less, for example, 25 wt % or less. When the amount is within the ranges above, an organic EL device with excellent luminescence color purity and high luminescence efficiency may be obtained. An amount of the compound of Formula (1) based on the total weight of an emission layer of an organic EL device to be described below may be the same as described above.

### Phosphorescent Complex

The material for an organic EL device according to an embodiment may further include a phosphorescent complex in addition to the compound of Formula (1). By including the phosphorescent complex, luminescence efficiency and device lifespan may be significantly improved. By using the compound of Formula (1) with the phosphorescent complex, luminescence efficiency and device lifespan may be significantly improved. While not wishing to be bound by theory, the reason is assumed as described below. The phosphorescent complex transfers energy to the compound of Formula (1) by the FRET mechanism. As a result, energy may be transferred with high efficiency from the phosphorescent complex to the compound of Formula (1). As such, it is assumed that as energy is transferred with high efficiency from the phosphorescent complex to the compound of Formula (1), the aforementioned effects may occur.

The phosphorescent complex may be, but is not particularly limited to, a metal complex in view of luminescence efficiency. In the same point of view, the phosphorescent complex may be a platinum complex or a palladium complex, for example, a platinum complex. Accordingly, in the material for an organic EL device according to an embodiment, the phosphorescent complex may be, for example, a platinum complex.

The phosphorescent complex may be, but is not particularly limited to, a compound having a structure of Formula (4), to improve luminescence color purity and luminescence efficiency:

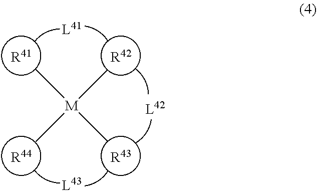

In Formula (4), M may be a metal ion having a coordination number of 4,
R⁴¹, R⁴², R⁴³, and R⁴⁴ may each independently be a substituted or unsubstituted hydrocarbon cyclic group or a substituted or unsubstituted heterocyclic group,
L⁴¹ may be a linking group linking R⁴¹ and R⁴²,
L⁴² may be a linking group linking R⁴² and R⁴³, and
L⁴³ may be a linking group linking R⁴³ and R⁴⁴
L⁴¹, L⁴² and L⁴³ may each independently be a single bond, *—O—*′, *—S—*′, *—N(R⁴⁵)—*′ or *—P(R⁴⁵)—*′. R⁴⁵ may be a phenyl group or a naphthyl group, and R⁴⁵ may optionally bond with adjacent R⁴¹, R⁴², R⁴³ or R⁴⁴ to form a substituted or unsubstituted heterocyclic group having 5 to 9 ring-forming atoms. The * and *′ are each a binding site with an adjacent atom.

In Formula (4), the hydrocarbon cyclic group refers to a group derived from at least one hydrocarbon ring. When the hydrocarbon cyclic group includes two or more hydrocarbon rings, the rings may be partially or entirely bonded via a single bond or condensed to each other. In addition, when the hydrocarbon cyclic group includes two or more hydrocarbon rings, one atom may serve as a ring-forming atom of any of the rings.

In addition, a heterocyclic group in Formula (4) is identical to the monovalent heterocyclic group described in (a8) of Formula (1), except for having a different valence number.

In Formula (4), substituents substituting the hydrocarbon cyclic group or the heterocyclic group may be, but are not particularly limited to, those mentioned above as substituents substituting the groups of (a3) to (a9) in Formula (1).

In Formula (4), M may be a platinum (Pt) ion or a palladium (Pd) ion, for example, a platinum (Pt) ion.

As the phosphorescent complex, a known compound may be used. For example, the platinum complex described in Tyler Fleetham et al., “Efficient “Pure” Blue OLEDs Employing Tetradentate Pt Complexes with a Narrow Spectral Bandwidth,” Advanced Materials, 2014, 26, 7116-7121, the platinum complex described in European Patent Laid-open Publication No. 3670520, the platinum complex and palladium complex described in Japanese Patent Laid-open Publication No. 2019-029500, or the platinum complex described in U.S. Patent Laid-open Publication No. 2015/0162552 may be used.

Hereinafter, the phosphorescent complex according to an embodiment will be described in detail. However, the disclosure is not limited to such examples.

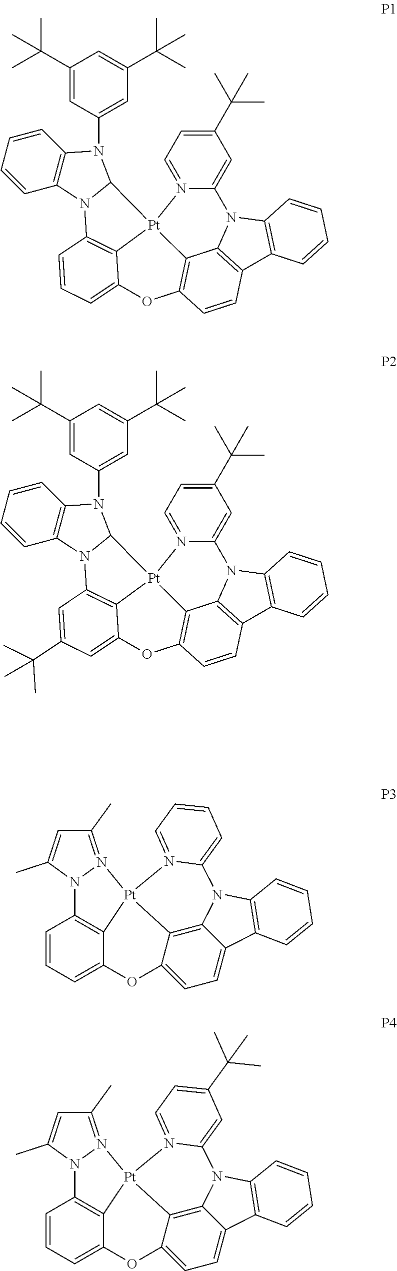

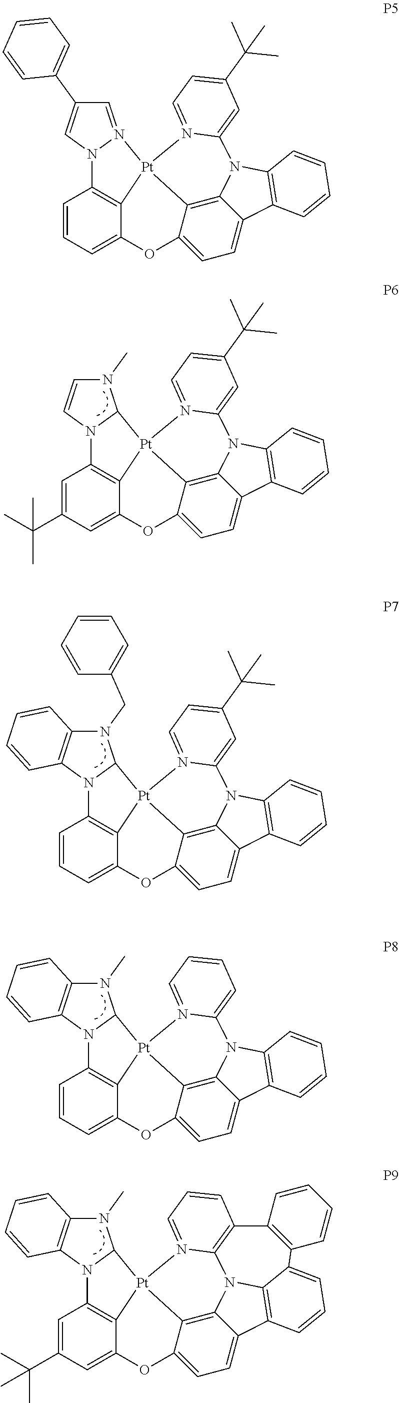

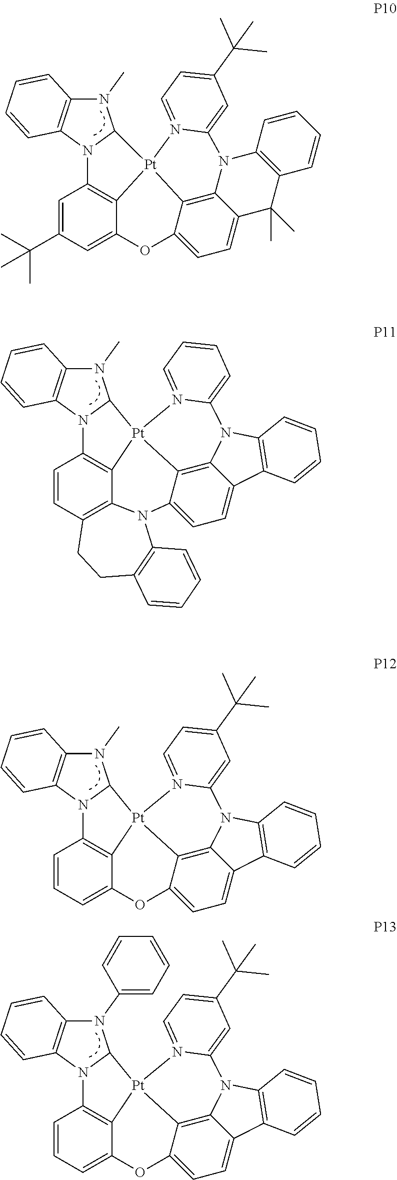

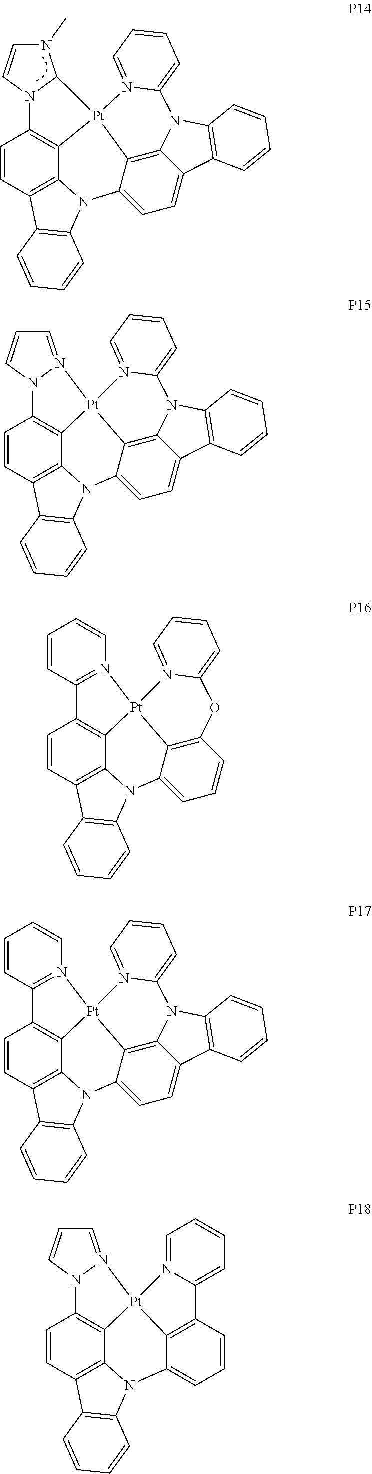

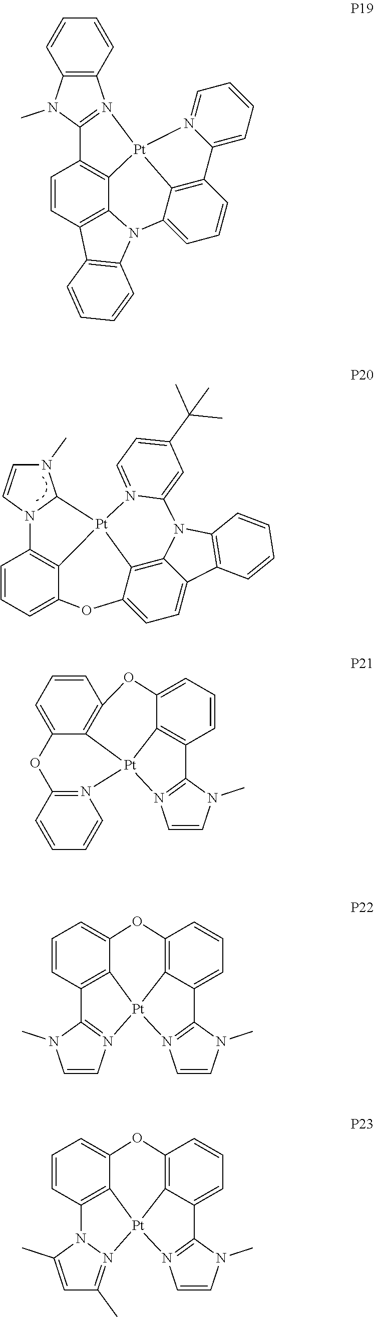

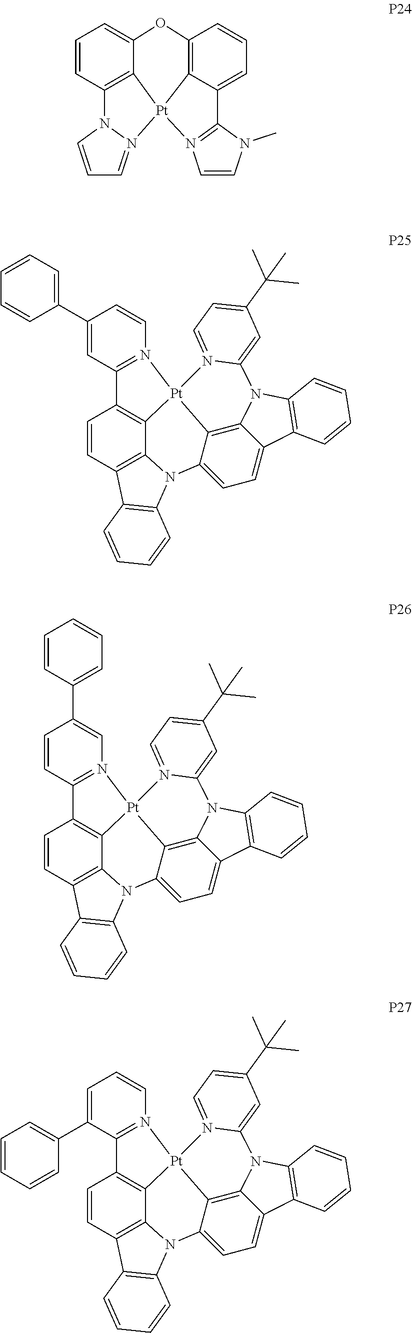

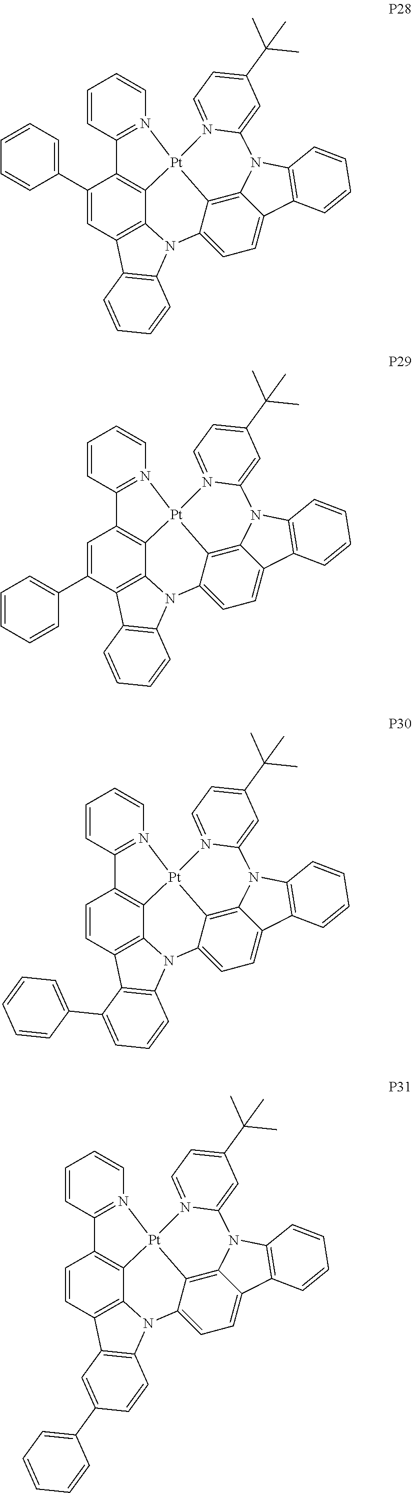

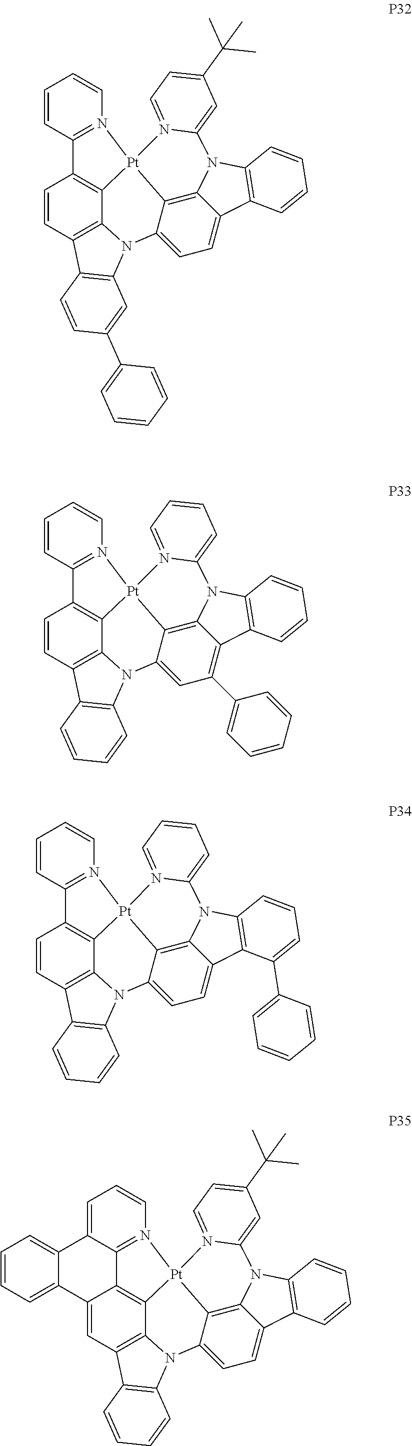

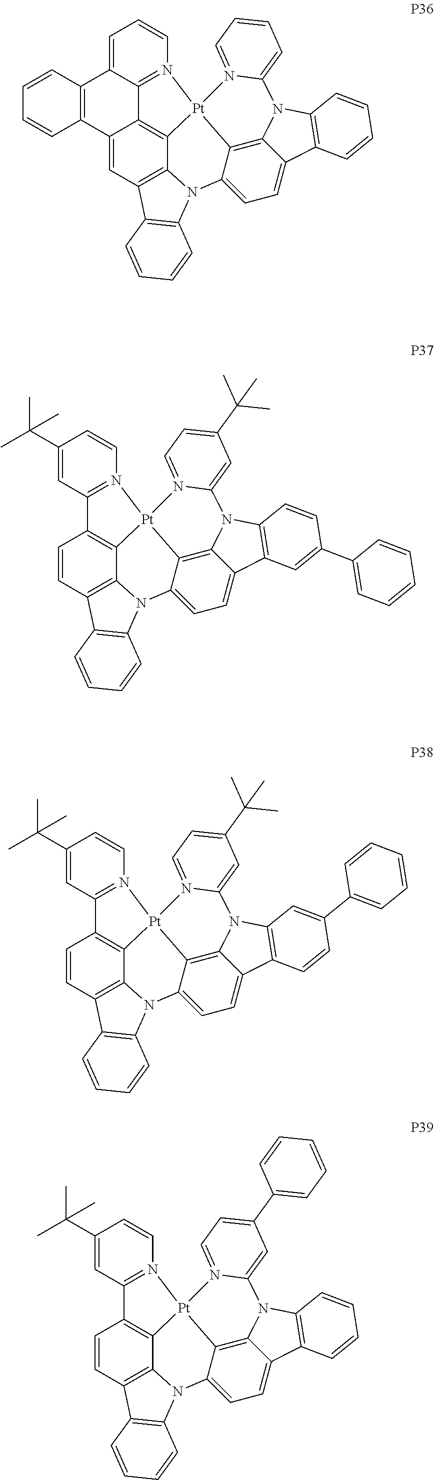

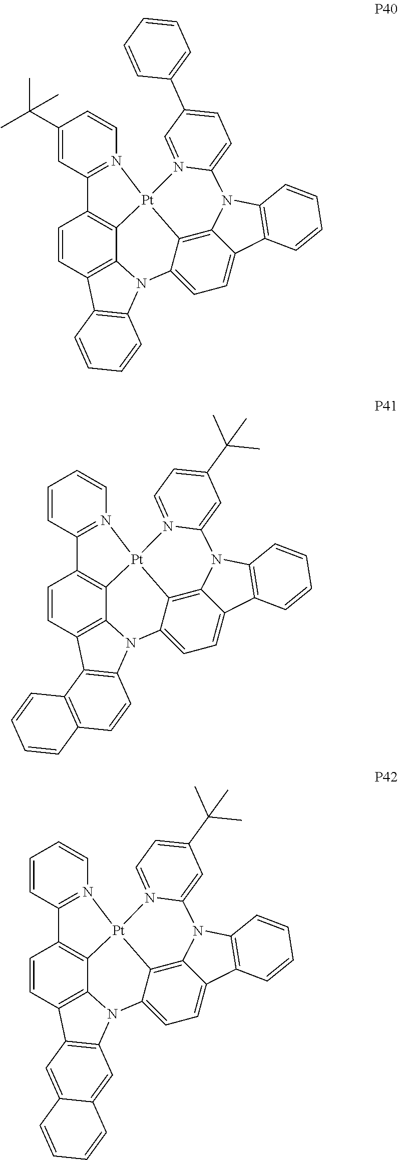

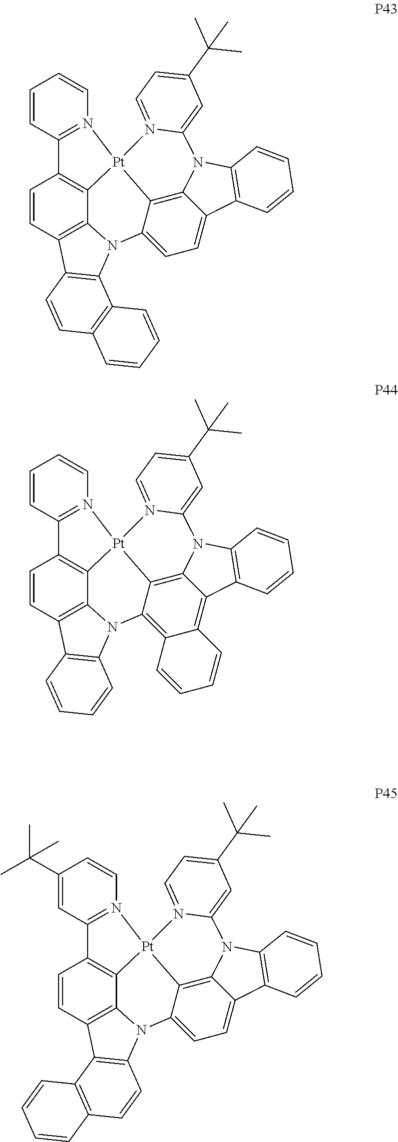

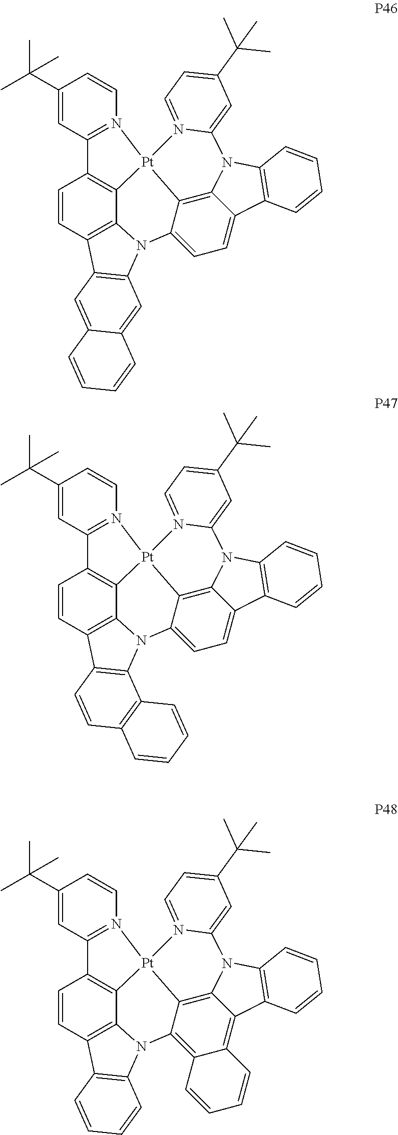

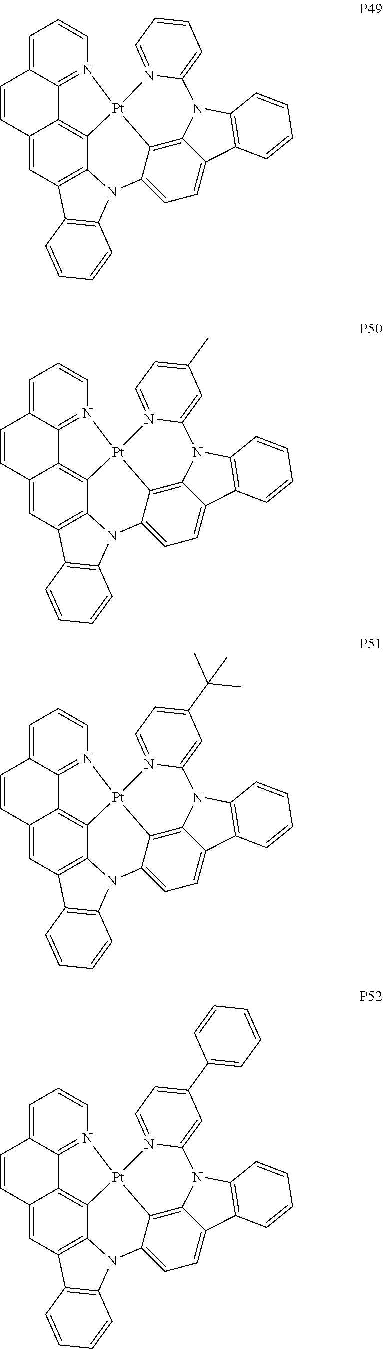

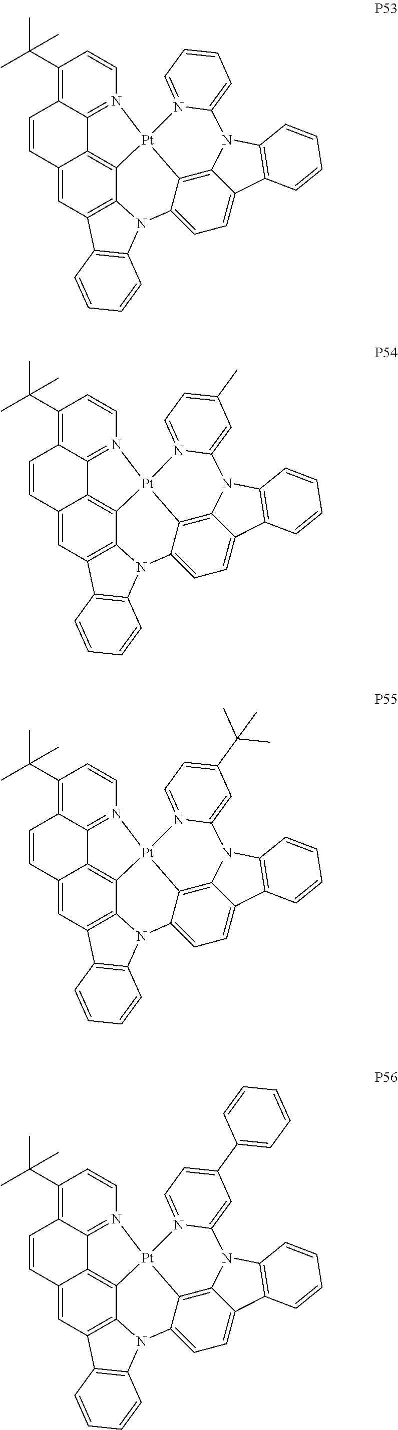

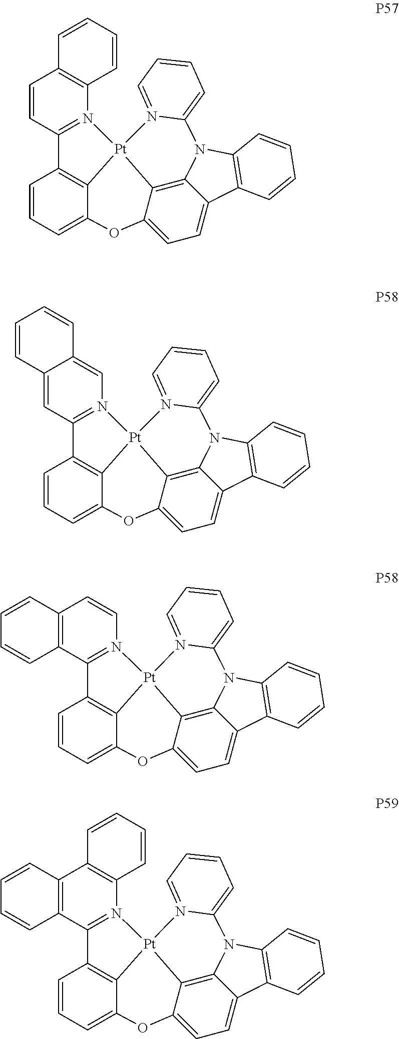

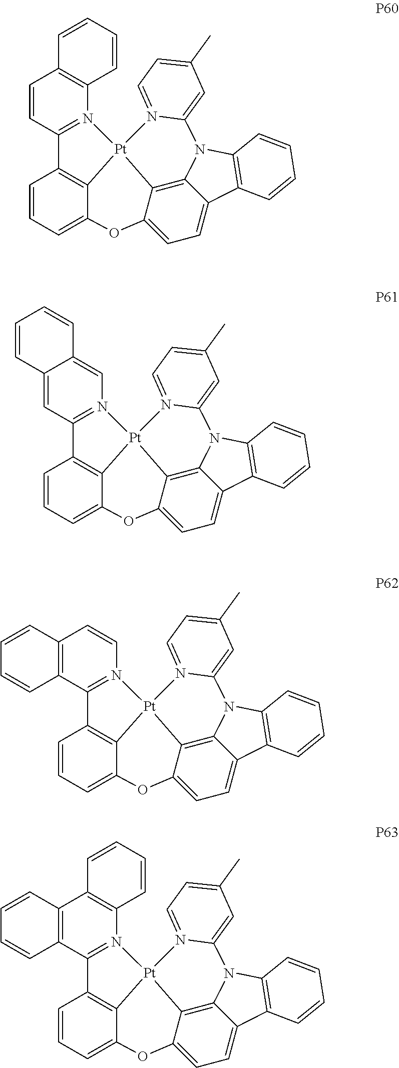

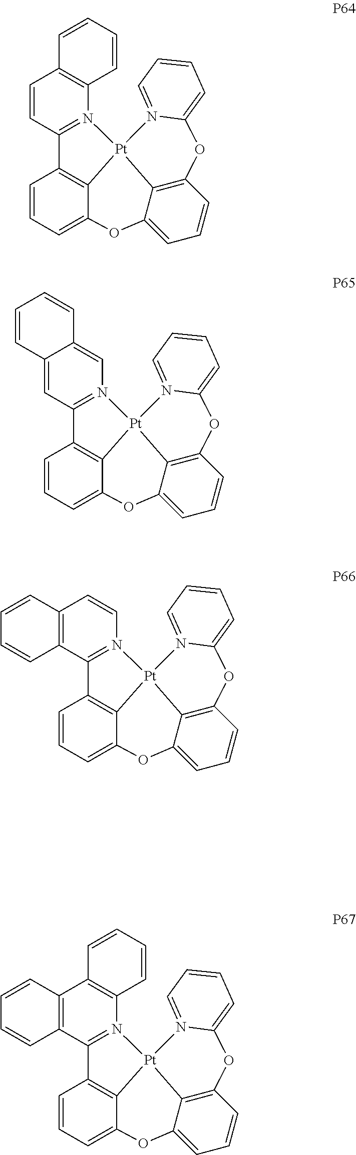

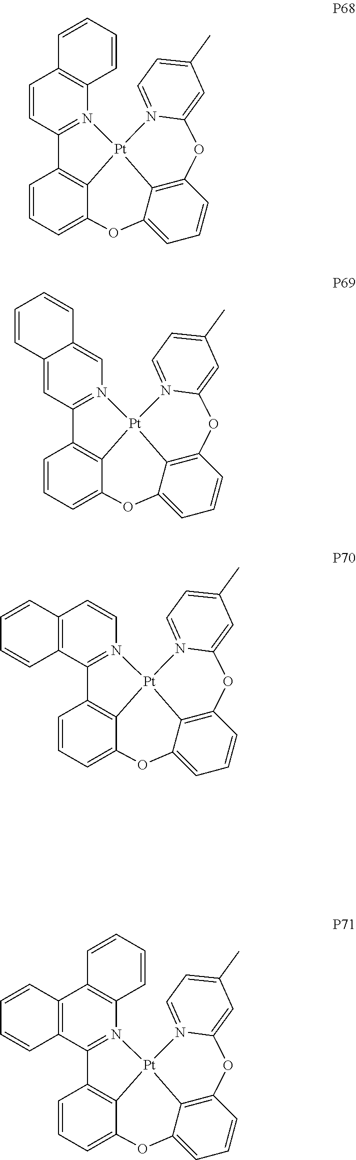

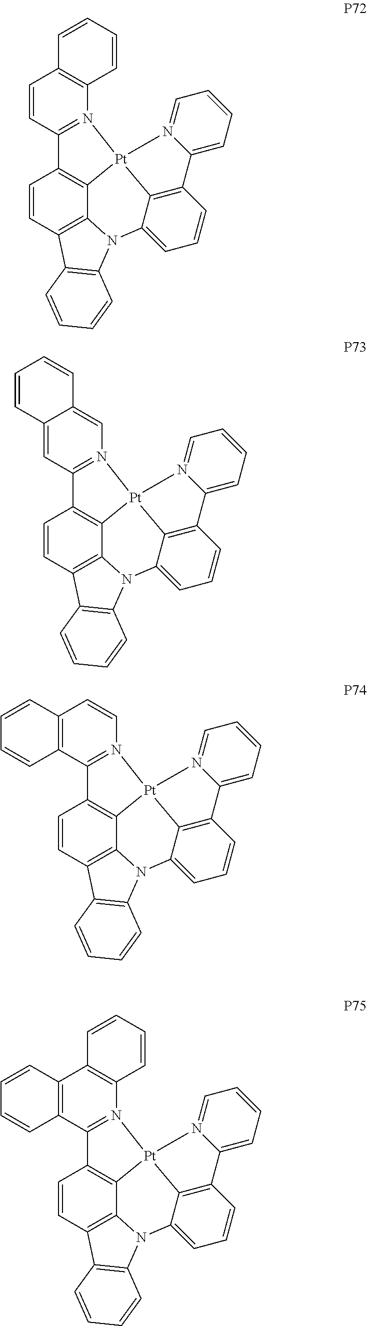

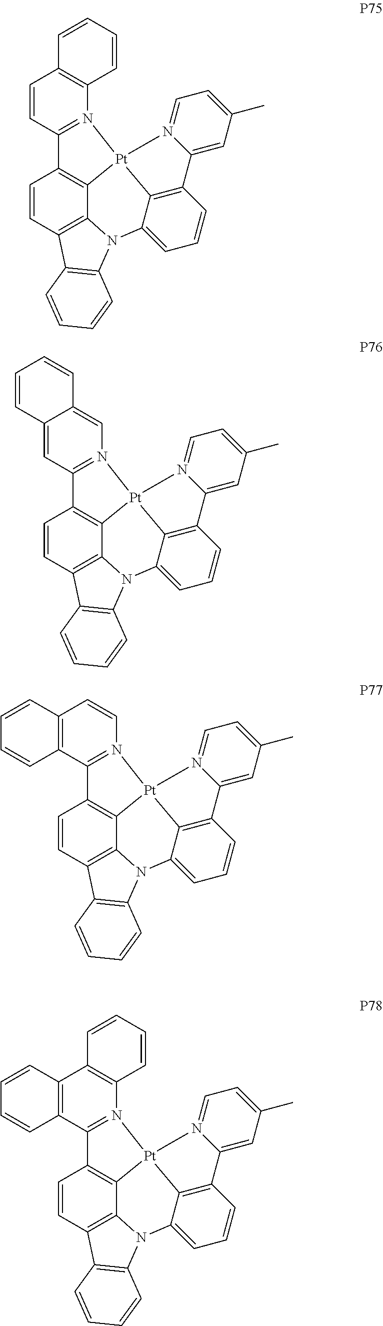

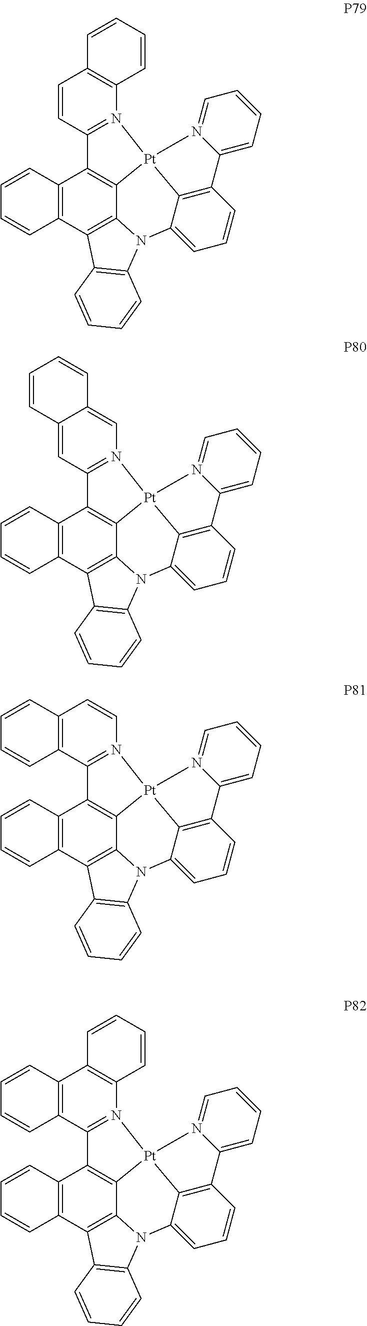

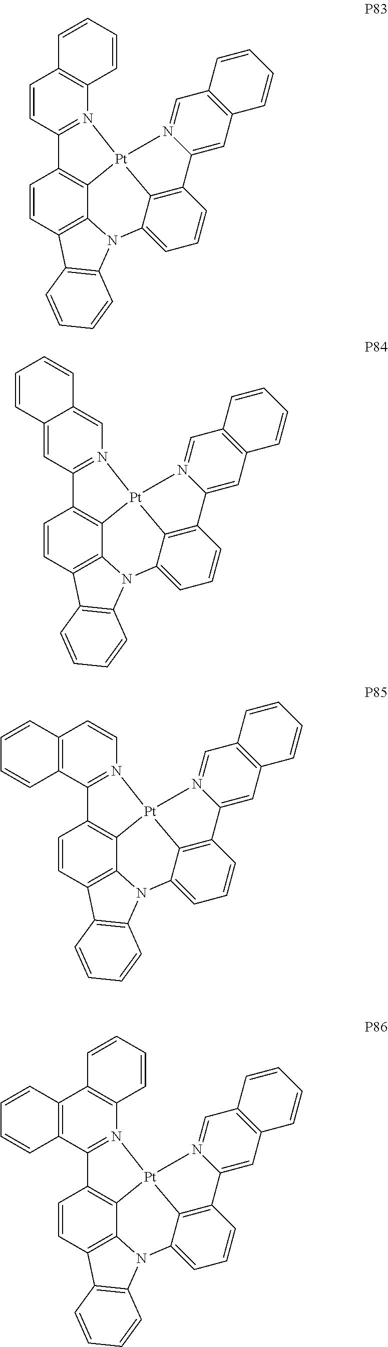

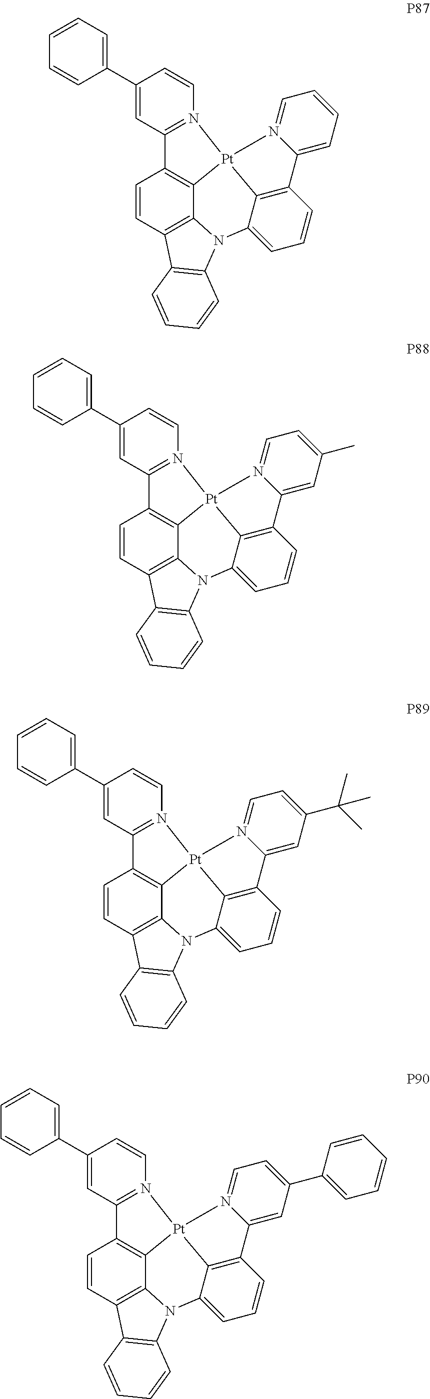

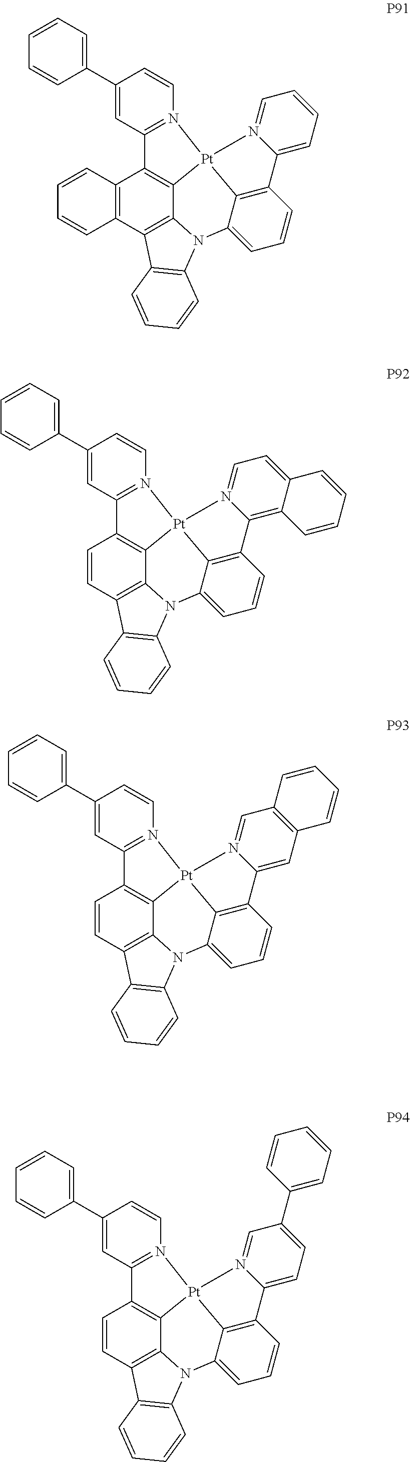

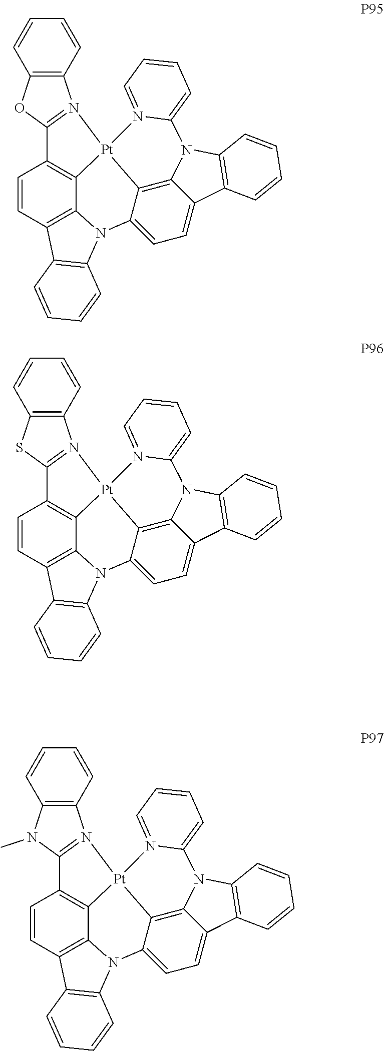

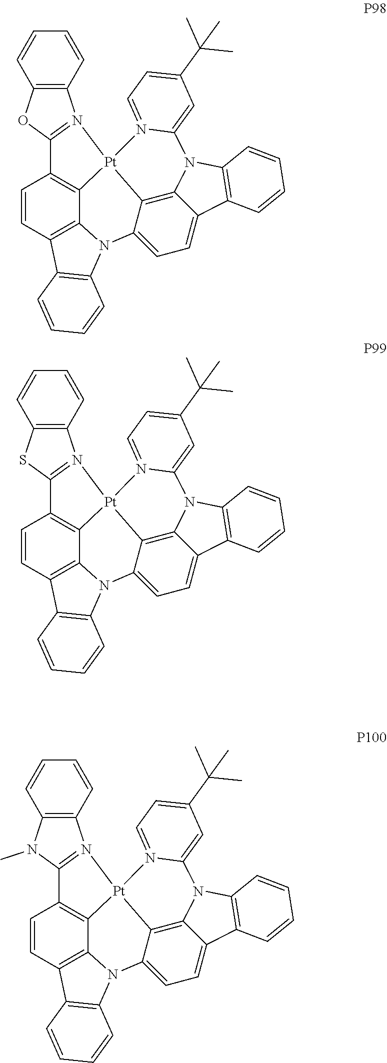

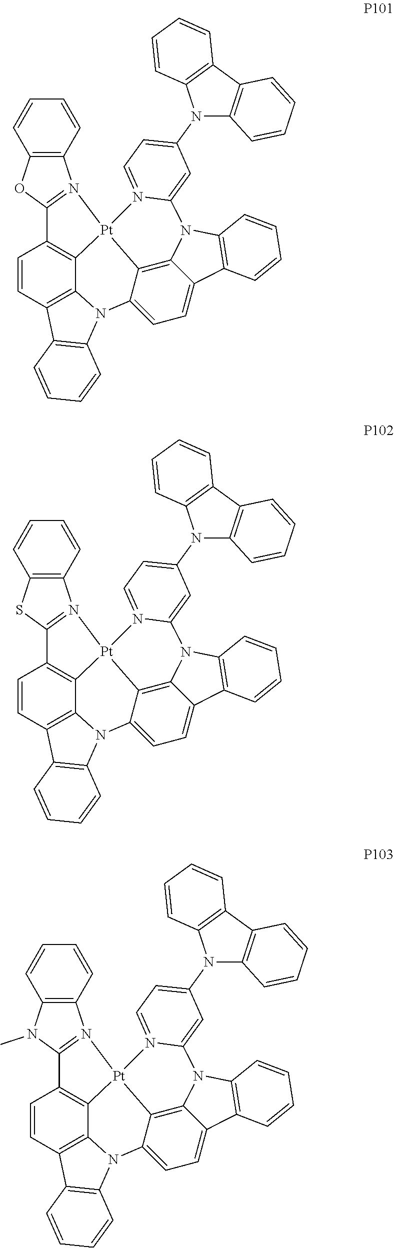

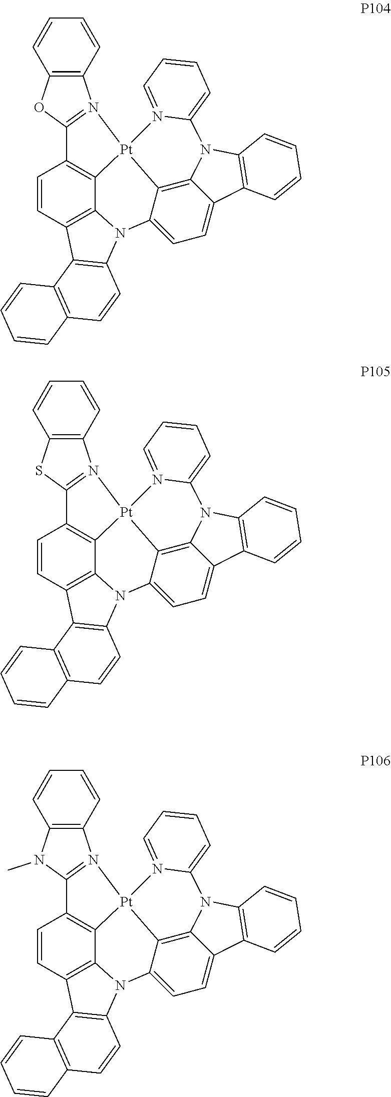

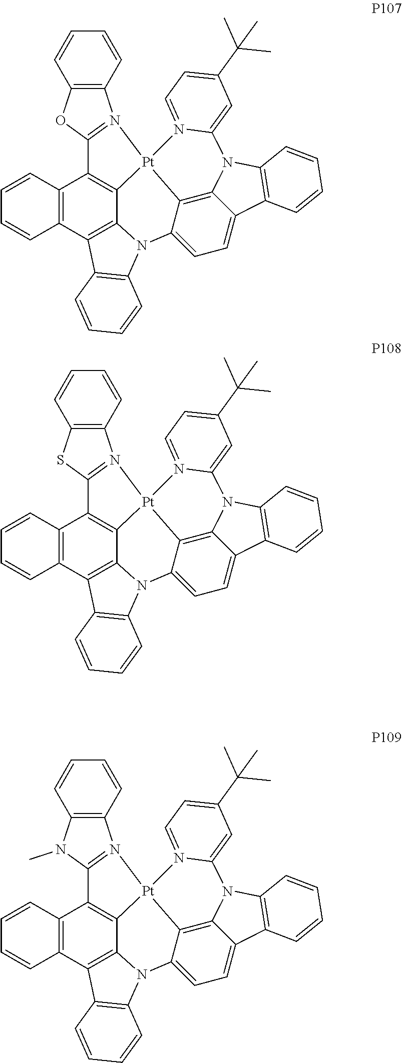

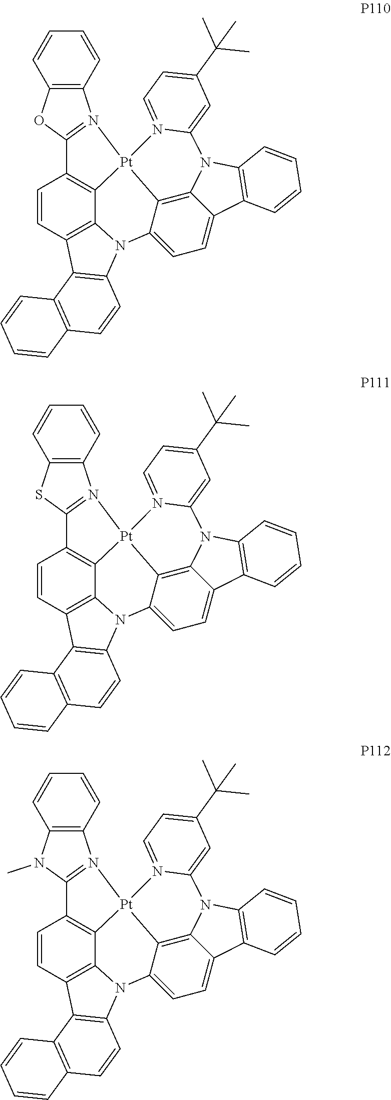

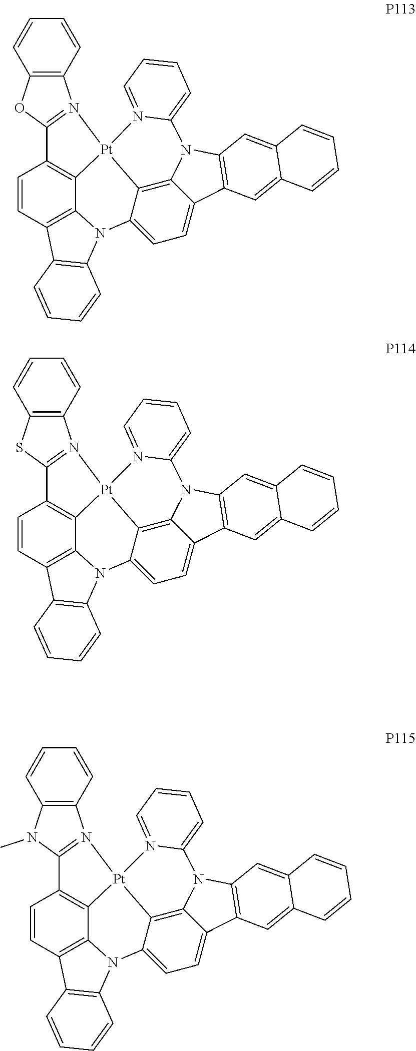

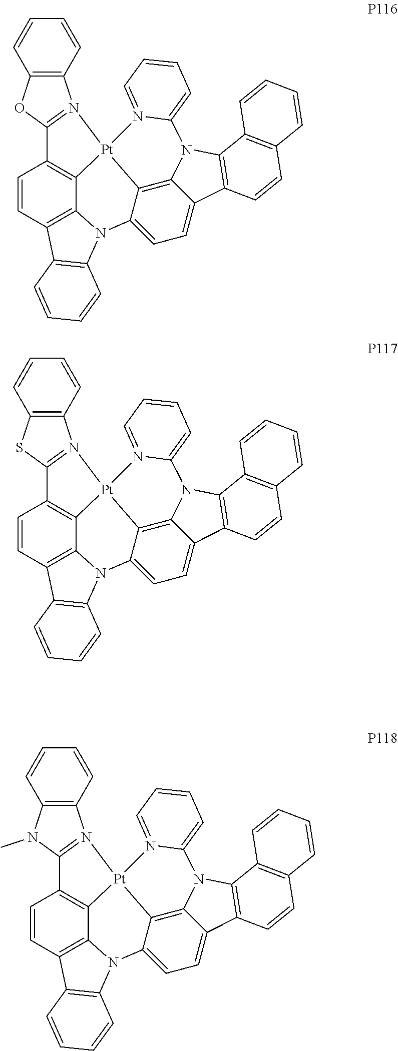

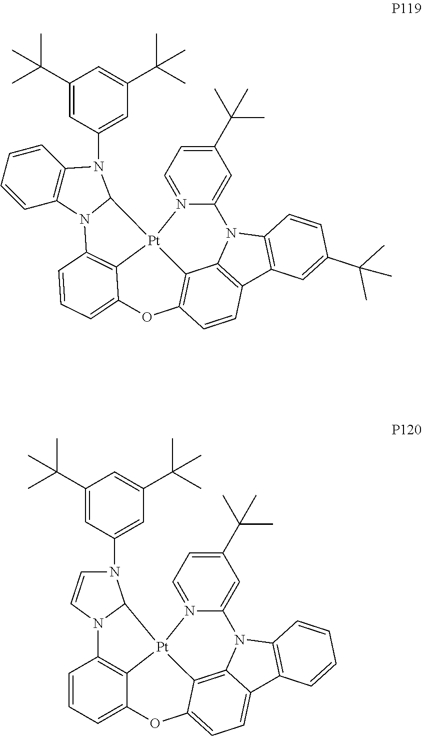

An amount of the phosphorescent complex may be, but is not particularly limited to, 0.1 wt % or more, for example, 0.2 wt % or more, based on the total weight of the material for an organic EL device (in particular, the material for an emission layer). In addition, the amount may be 0.5 wt % or more, for example, 1 wt % or more. In addition, the amount may be 3 wt % or more, for example, 5 wt % or more. When the amount is within the ranges above, an organic EL device with excellent luminescence color purity and high luminescence efficiency may be obtained. In addition, an amount of the phosphorescent complex may be, but is not particularly limited to, 50 wt % or less based on the total weight of the material for an organic EL device (in particular, the material for an emission layer). In addition, the amount may be 40 wt % or less, for example, 30 wt % or less. When the amount is within the ranges above, an organic EL device with excellent luminescence color purity and high luminescence efficiency may be obtained. An amount of the phosphorescent complex based on the total weight of an emission layer of an organic EL device to be described below may be the same as described above.

When the material for an organic EL device (in particular, the material for an emission layer) includes the phosphorescent complex, an amount of the phosphorescent complex may be 100 parts by weight or more based on 100 parts by weight of the compound of Formula (1). In addition, the amount may be 150 parts by weight or more, for example, 200 parts by weight or more, based on 100 parts by weight of the compound of Formula (1). When the amount is within the ranges above, an organic EL device with excellent luminescence color purity and high luminescence efficiency may be obtained. In addition, an amount of the phosphorescent complex may be, but is not particularly limited to, 10,000 parts by weight or less based on 100 parts by weight of the compound of Formula (1). In addition, the amount may be 7,500 parts by weight or less, for example, 5,000 parts by weight or less, based on 100 parts by weight of the compound of Formula (1). When the amount is within the ranges above, an organic EL device with excellent luminescence color purity and high luminescence efficiency may be obtained. An amount (parts by weight) of the phosphorescent complex based on 100 parts by weight of the compound of Formula (1) in an emission layer of an organic EL device to be described below may be the same as described above.

### Host Material

The material for an organic EL device according to an embodiment may further include a host material in addition to the compound of Formula (1). By using the compound of Formula (1) as a dopant material in combination with the host material, an organic EL device may have excellent luminescence efficiency and device lifespan.

The host material may be, but is not particularly limited to, a known host material. For example, the known host material may be an anthracene derivative, a pyrene derivative, a fluoranthene derivative, a chrysene derivative, a dihydrobenzoanthracene derivative, or a triphenylene derivative. As an anthracene derivative, 9-(1-naphthyl)-10-(2-naphthyl) anthracene (Compound HT4) may be used.

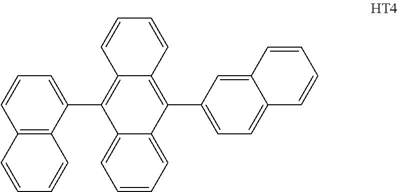 [SMILES: c1ccc2cc(-c3c4ccccc4c(-c4cccc5ccccc45)c4ccccc34)ccc2c1]

Preferred examples of the host material may include a compound with a carbazole ring structure (excluding the compound represented by Formula (1)), a compound with a ring structure wherein at least one ring-forming carbon atom of the carbazole ring is substituted into a nitrogen atom (excluding the compound represented by Formula (1) and the compound with the carbazole ring structure), or a compound with the triazine ring structure (excluding the compound represented by Formula (1), the compound with the carbazole ring structure, and the compound with a ring structure wherein at least one ring-forming carbon atom of the carbazole ring is substituted into a nitrogen atom). In an embodiment, the host material may be a compound with a carbazole ring structure. By using such compounds as host materials, efficient energy transfer in an emission layer may be enhanced. In addition, the balance of carrier mobility between electrons and holes may be improved. A hydrogen atom bonded to a ring-forming atom constituting a carbazole ring structure, a ring structure wherein at least one ring-forming carbon atom of a carbazole ring is substituted with a nitrogen atom, and a triazine ring structure of the above compounds may be substituted with other atoms or substituents. In addition, two or more of such substituents may constitute a ring structure.

The compound with the carbazole ring structure or the compound having the ring structure in which at least one ring-forming carbon atom of a carbazole ring is substituted with a nitrogen atom may be, but is not particularly limited to, a compound with a structure represented by Formula (5):

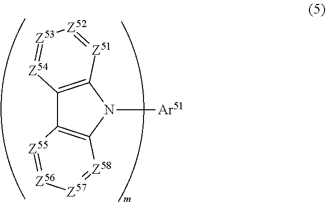

wherein, in Formula (5),
Z⁵¹ may be CH, CR⁵¹, or N,
Z⁵² may be CH, CR⁵², or N,
Z⁵³ may be CH, CR⁵³, or N,
Z⁵⁴ may be CH, CR⁵⁴, or N,
Z⁵⁵ may be CH, CR⁵⁵, or N,
Z⁵⁶ may be CH, CR⁵⁶, or N,
Z⁵⁷ may be CH, CR⁵⁷, or N,
Z⁵⁸ may be CH, CR⁵⁸, or N,
R⁵¹ to R⁵⁸ may each independently be any one group selected from (5a) to (5h):
(5a) a cyano group;
(5b) a substituted or unsubstituted alkyl group having 1 to 20 carbon atoms;
(5c) a substituted or unsubstituted alkoxy group having 1 to 20 carbon atoms;
(5d) a substituted or unsubstituted arylamino group having 6 to 20 carbon atoms;
(5e) a substituted or unsubstituted phosphoryl group (a —POH₂ group);
(5f) a substituted or unsubstituted silyl group (a —SiH₃ group);
(5g) a substituted or unsubstituted monovalent aromatic hydrocarbon group; and
(5h) a substituted or unsubstituted monovalent heterocyclic group,
wherein Ar⁵¹ may be a group including at least one of the aromatic hydrocarbon group and the heterocyclic group,
m may be 1, 2, 3, 4, 5, or 6, and
wherein R⁵¹ and R⁵², R⁵² and R⁵³, R⁵³ and R⁵⁴, R⁵⁵ and R⁵⁶, R⁵⁶ and R⁵⁷, or R⁵⁷ and R⁵⁸ may optionally form an aliphatic hydrocarbon ring, an aromatic hydrocarbon ring, or a hetero ring, each including bonded carbon atoms.

In Formula (5), descriptions of (5b) to (5d), (5g), and (5h) are respectively the same as the descriptions of (a3) to (a5), (a7), and (a8) of Formula (1).

An aromatic hydrocarbon group in Ar⁵¹ is identical to the monovalent aromatic hydrocarbon group described in (a7) of Formula (1), except for having a different valence number.

In addition, a heterocyclic group in Ar⁵¹ is identical to the monovalent heterocyclic group described in (a8) of Formula (1), except for having a different valence number.

In an embodiment, in Formula (5), Z⁵¹ to Z⁵⁸ may not be N or only one of Z⁵¹ to Z⁵⁸ may be N. In an embodiment, Z⁵¹ to Z⁵⁸ may not be N.

In Formula (5), when the groups of (5b) to (5h) are substituted groups, substituents substituting such groups are not particularly limited. For example, the substituents may be the groups of (5a) to (5h). Examples of the substituents substituting such groups may be, but not limited to, a cyano group, an unsubstituted alkyl group having 1 to 20 carbon atoms, an alkoxy group having 1 to 20 carbon atoms substituted with a monovalent aromatic hydrocarbon group having 6 to 30 carbon atoms substituted with an unsubstituted alkenyl group having 2 to 30 carbon atoms, a substituted or unsubstituted arylamino group having 6 to 20 carbon atoms, an unsubstituted monovalent aromatic hydrocarbon group having 6 to 30 carbon atoms, a monovalent aromatic hydrocarbon group having 6 to 30 carbon atoms substituted with a cyano group, a monovalent aromatic hydrocarbon group having 6 to 30 carbon atoms substituted with an unsubstituted alkenyl group having 2 to 30 carbon atoms, a monovalent aromatic hydrocarbon group having 6 to 30 carbon atoms substituted with an unsubstituted amino group having 6 to 20 carbon atoms, an unsubstituted monovalent heterocyclic group having 3 to 30 ring-forming atoms, a monovalent heterocyclic group having 3 to 30 ring-forming atoms substituted with an unsubstituted monovalent aromatic hydrocarbon group having 6 to 30 carbon atoms, and the like.

In Formula (5), Ar⁵¹ may be, but is not particularly limited to, a group with at least one of the aromatic hydrocarbon group and the heterocyclic group. Examples of Ar⁵¹ may include, for example, a substituted or unsubstituted aromatic hydrocarbon group, a substituted or unsubstituted heterocyclic group, a group in which at least one substituted or unsubstituted aromatic hydrocarbon group is bonded to at least one substituted or unsubstituted heterocyclic group via a single bond, or a group in which at least two substituted or unsubstituted aromatic hydrocarbon groups or substituted or unsubstituted heterocyclic groups are bonded to each other via a linking group other than the at least two groups.

In this regard, in the group in which at least two substituted or unsubstituted aromatic hydrocarbon groups or substituted or unsubstituted heterocyclic groups are bonded to each other via a linking group other than the at least two groups, the linking group is not particularly limited. Examples of the linking group may include a Si group, a N group, a P═O group, a S(═O)═O group, a C═O group, and the like.

In Formula (5), when groups constituting Ar⁵¹ are substituted groups, substituents substituting such groups are not particularly limited. For example, the substituents may be the groups of (5a) to (5h). Examples of the substituents substituting such groups may include, but are not particularly limited to, a cyano group, an unsubstituted alkoxy group having 1 to 20 carbon atoms, a monovalent heterocyclic group having 3 to 30 ring-forming atoms substituted with an unsubstituted alkoxy group having 1 to 20 carbon atoms, and the like.

In the substituents of the groups of (5b) to (5h) or the substituent of the groups constituting Ar⁵¹, an alkyl group having 1 to 20 carbon atoms, an alkoxy group having 1 to 20 carbon atoms, an arylamino group having 6 to 20 carbon atoms, a monovalent aromatic hydrocarbon group having 6 to 30 carbon atoms, a monovalent heterocyclic group having 3 to 30 ring-forming atoms are respectively the same as described in connection with the groups of (a3) to (a9) of Formula (1).

The alkenyl group having 2 to 30 carbon atoms in the substituents of the groups of (5c) to (5h) or the substituents of the groups constituting Ar⁵¹ is not particularly limited, and may be linear, branched, or cyclic. Examples of the alkenyl group may include, but are not particularly limited to, a vinyl group, a 2-propenyl group, a 2-butenyl group, a 3-butenyl group, a 1-methyl-2-propenyl group, a 2-methyl-2-propenyl group, a 2-pentenyl group, a 3-pentenyl group, a 4-pentenyl group, a 1-methyl-2-butenyl group, a 2-methyl-2-butenyl group, a 3-methyl-2-butenyl group, a 1-methyl-3-butenyl group, a 2-methyl-3-butenyl group, a 3-methyl-3-butenyl group, a 1,1-dimethyl-2-propenyl group, a 1,2-dimethyl-2-propenyl group, a 1-ethyl-2-propenyl group, and the like.
m may be 1, 2, 3, or 4, and may be 2.

Hereinafter, as the host material according to an embodiment, the compound with the carbazole ring structure and the compound with the ring structure in which at least one ring-forming carbon atom of a carbazole ring is substituted with a nitrogen atom will be described in detail. However, the disclosure is not limited to such examples.

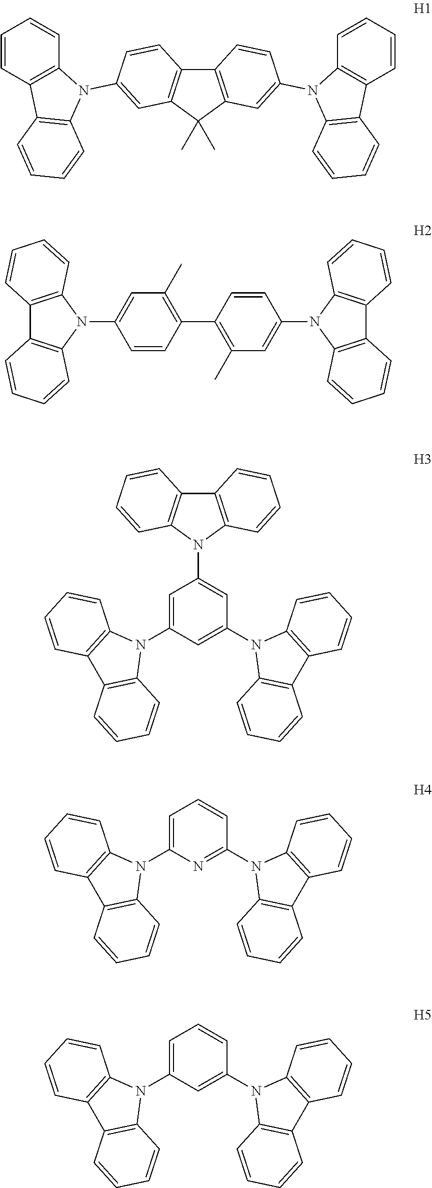 [SMILES: CC1(C)c2cc(-n3c4ccccc4c4ccccc43)ccc2-c2ccc(-n3c4ccccc4c4ccccc43)cc21 ; Cc1cc(-n2c3ccccc3c3ccccc32)ccc1-c1ccc(-n2c3ccccc3c3ccccc32)cc1C ; c1ccc2c(c1)c1ccccc1n2-c1cc(-n2c3ccccc3c3ccccc32)cc(-n2c3ccccc3c3ccccc32)c1 ; c1cc(-n2c3ccccc3c3ccccc32)nc(-n2c3ccccc3c3ccccc32)c1 ; c1cc(-n2c3ccccc3c3ccccc32)cc(-n2c3ccccc3c3ccccc32)c1]

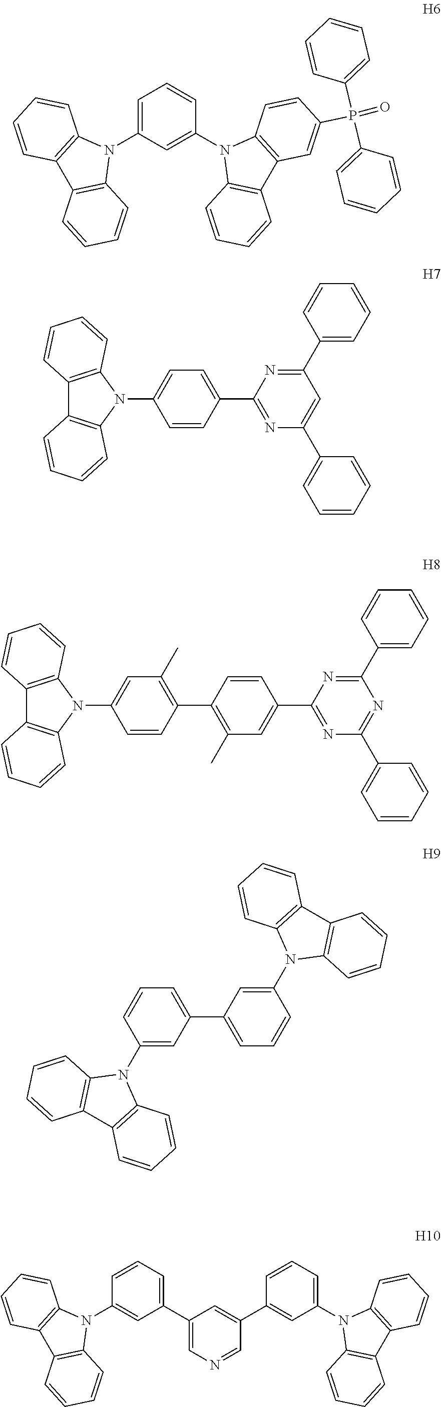 [SMILES: O=P(c1ccccc1)(c1ccccc1)c1ccc2c(c1)c1ccccc1n2-c1cccc(-n2c3ccccc3c3ccccc32)c1 ; c1ccc(-c2cc(-c3ccccc3)nc(-c3ccc(-n4c5ccccc5c5ccccc54)cc3)n2)cc1 ; Cc1cc(-c2nc(-c3ccccc3)nc(-c3ccccc3)n2)ccc1-c1ccc(-n2c3ccccc3c3ccccc32)cc1C ; c1cc(-c2cccc(-n3c4ccccc4c4ccccc43)c2)cc(-n2c3ccccc3c3ccccc32)c1 ; c1cc(-c2cncc(-c3cccc(-n4c5ccccc5c5ccccc54)c3)c2)cc(-n2c3ccccc3c3ccccc32)c1]

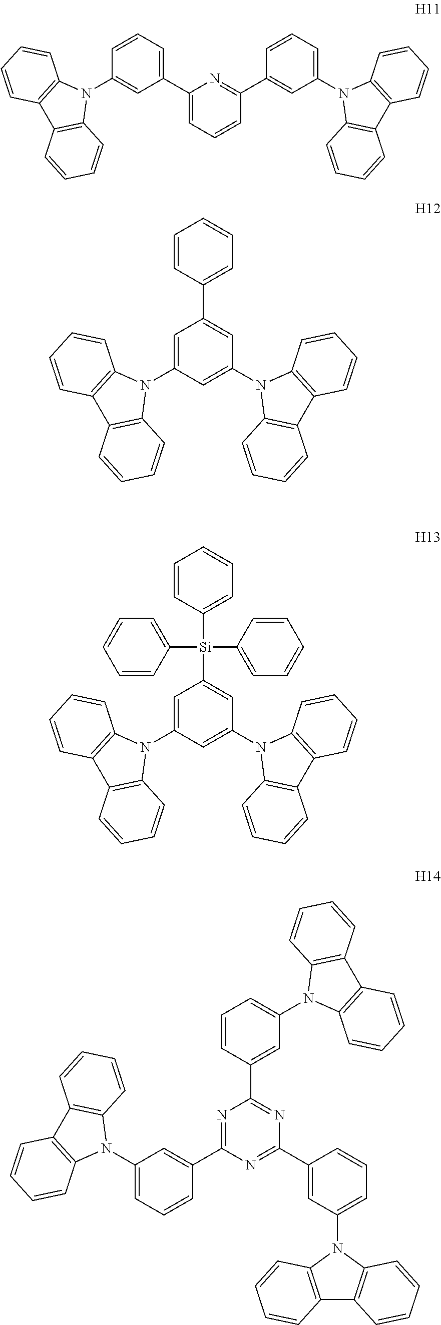 [SMILES: c1cc(-c2cccc(-c3cccc(-n4c5ccccc5c5ccccc54)c3)n2)cc(-n2c3ccccc3c3ccccc32)c1 ; c1ccc(-c2cc(-n3c4ccccc4c4ccccc43)cc(-n3c4ccccc4c4ccccc43)c2)cc1 ; c1ccc([Si](c2ccccc2)(c2ccccc2)c2cc(-n3c4ccccc4c4ccccc43)cc(-n3c4ccccc4c4ccccc43)c2)cc1 ; c1cc(-c2nc(-c3cccc(-n4c5ccccc5c5ccccc54)c3)nc(-c3cccc(-n4c5ccccc5c5ccccc54)c3)n2)cc(-n2c3ccccc3c3ccccc32)c1]

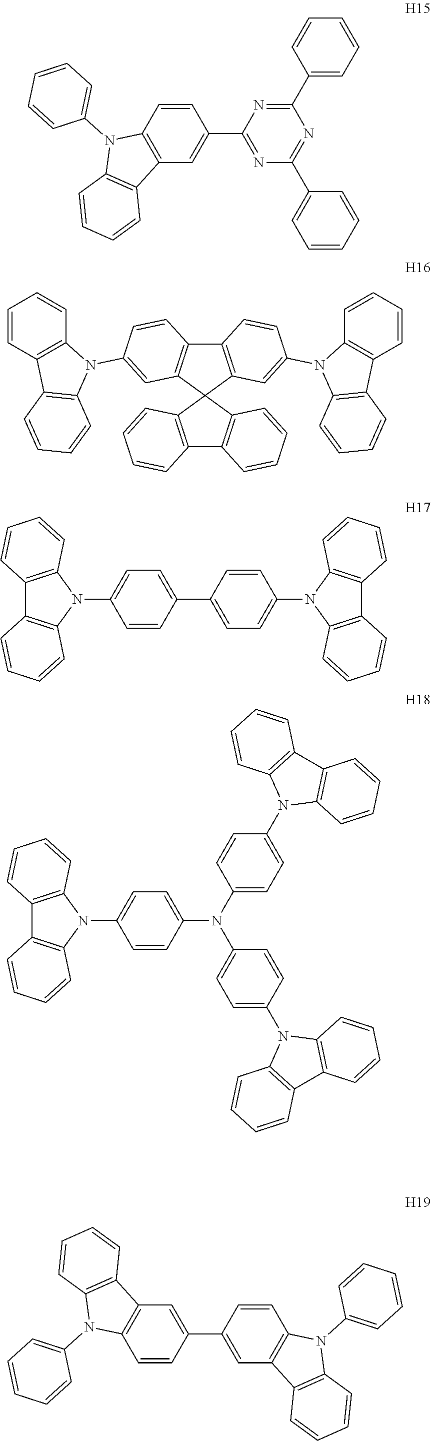 [SMILES: c1ccc(-c2nc(-c3ccccc3)nc(-c3ccc4c(c3)c3ccccc3n4-c3ccccc3)n2)cc1 ; c1ccc2c(c1)-c1ccccc1C21c2cc(-n3c4ccccc4c4ccccc43)ccc2-c2ccc(-n3c4ccccc4c4ccccc43)cc21 ; c1ccc2c(c1)c1ccccc1n2-c1ccc(-c2ccc(-n3c4ccccc4c4ccccc43)cc2)cc1 ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1 ; c1ccc(-n2c3ccccc3c3cc(-c4ccc5c(c4)c4ccccc4n5-c4ccccc4)ccc32)cc1]

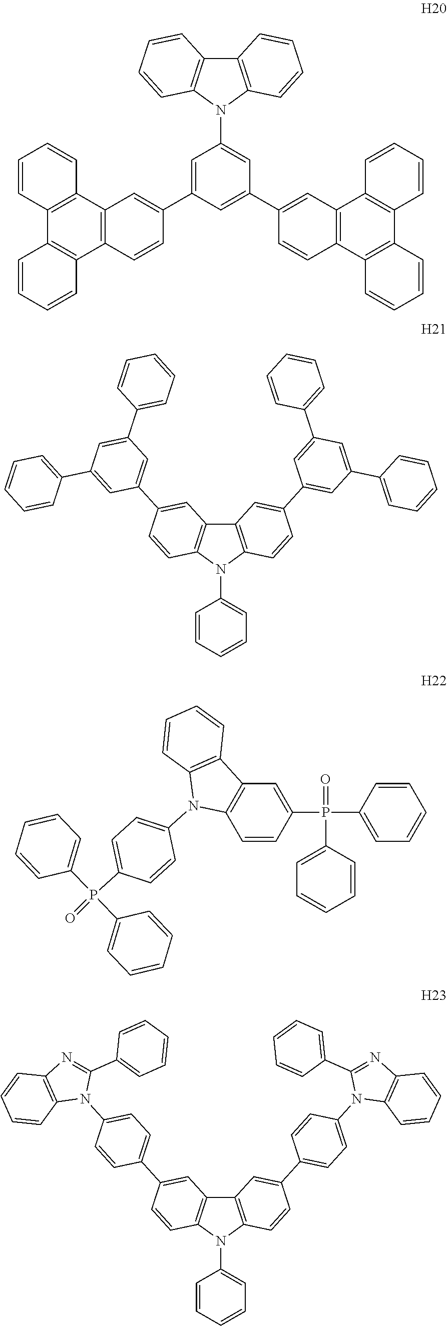 [SMILES: c1ccc2c(c1)c1ccccc1c1cc(-c3cc(-c4ccc5c6ccccc6c6ccccc6c5c4)cc(-n4c5ccccc5c5ccccc54)c3)ccc21 ; c1ccc(-c2cc(-c3ccccc3)cc(-c3ccc4c(c3)c3cc(-c5cc(-c6ccccc6)cc(-c6ccccc6)c5)ccc3n4-c3ccccc3)c2)cc1 ; O=P(c1ccccc1)(c1ccccc1)c1ccc(-n2c3ccccc3c3cc(P(=O)(c4ccccc4)c4ccccc4)ccc32)cc1 ; c1ccc(-c2nc3ccccc3n2-c2ccc(-c3ccc4c(c3)c3cc(-c5ccc(-n6c(-c7ccccc7)nc7ccccc76)cc5)ccc3n4-c3ccccc3)cc2)cc1]

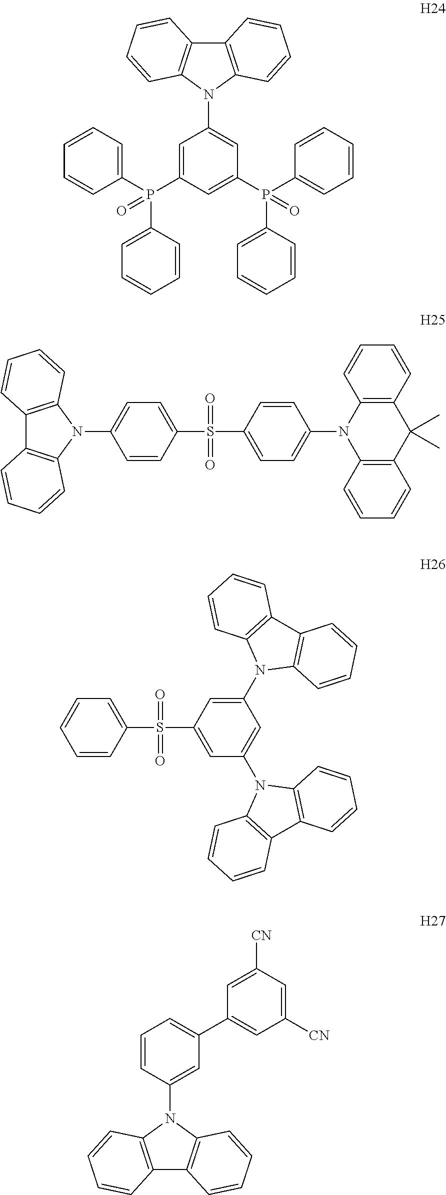 [SMILES: O=P(c1ccccc1)(c1ccccc1)c1cc(-n2c3ccccc3c3ccccc32)cc(P(=O)(c2ccccc2)c2ccccc2)c1 ; CC1(C)c2ccccc2N(c2ccc(S(=O)(=O)c3ccc(-n4c5ccccc5c5ccccc54)cc3)cc2)c2ccccc21 ; O=S(=O)(c1ccccc1)c1cc(-n2c3ccccc3c3ccccc32)cc(-n2c3ccccc3c3ccccc32)c1 ; N#Cc1cc(C#N)cc(-c2cccc(-n3c4ccccc4c4ccccc43)c2)c1]

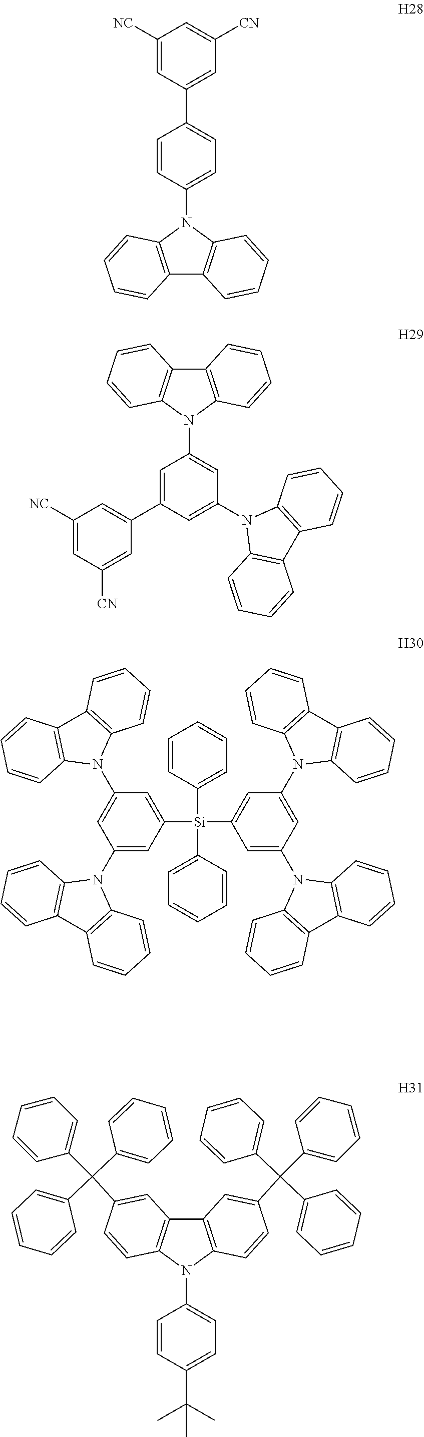

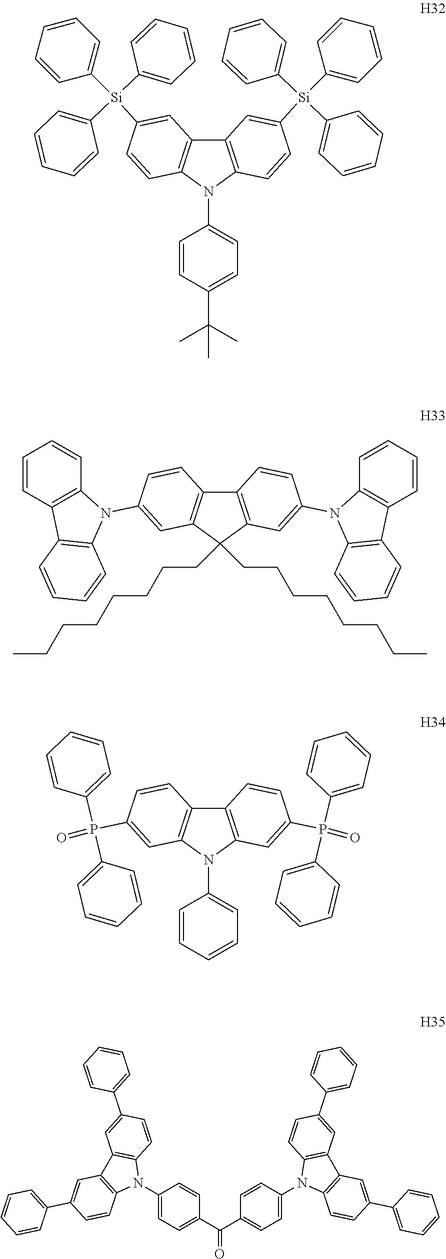

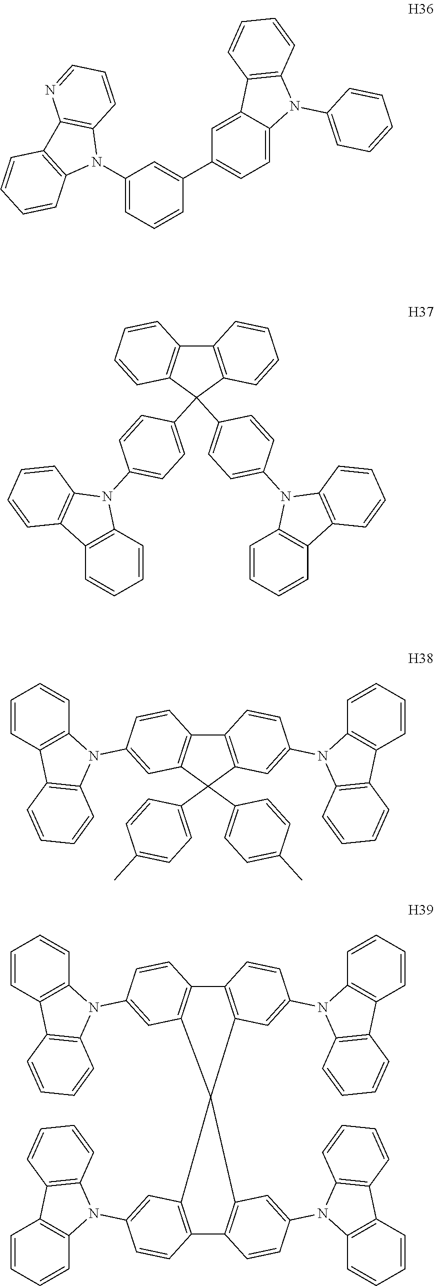

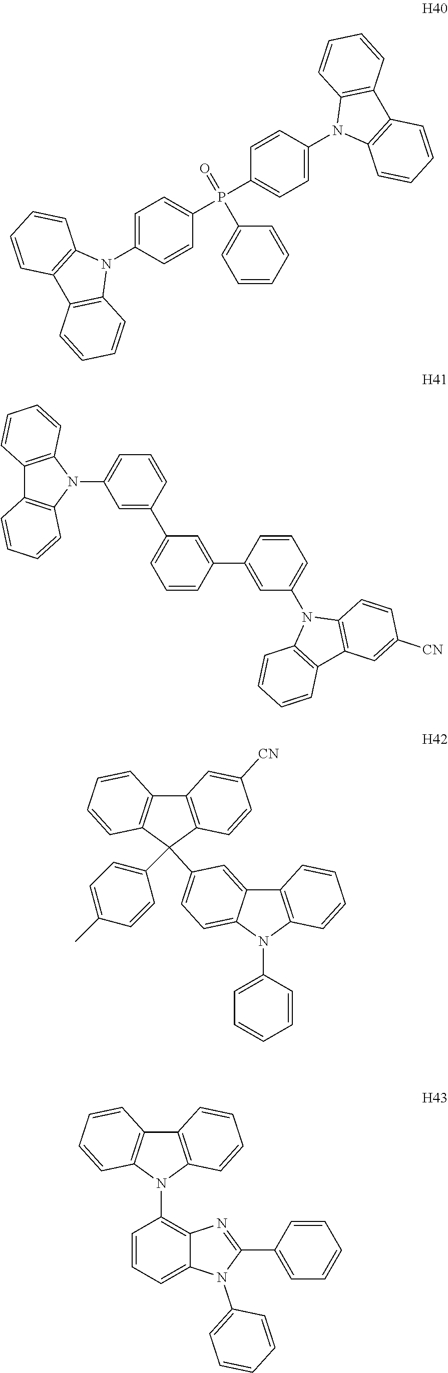 [SMILES: O=P(c1ccccc1)(c1ccc(-n2c3ccccc3c3ccccc32)cc1)c1ccc(-n2c3ccccc3c3ccccc32)cc1 ; N#Cc1ccc2c(c1)c1ccccc1n2-c1cccc(-c2cccc(-c3cccc(-n4c5ccccc5c5ccccc54)c3)c2)c1 ; Cc1ccc(C2(c3ccc4c(c3)c3ccccc3n4-c3ccccc3)c3ccccc3-c3cc(C#N)ccc32)cc1 ; c1ccc(-c2nc3c(-n4c5ccccc5c5ccccc54)cccc3n2-c2ccccc2)cc1]

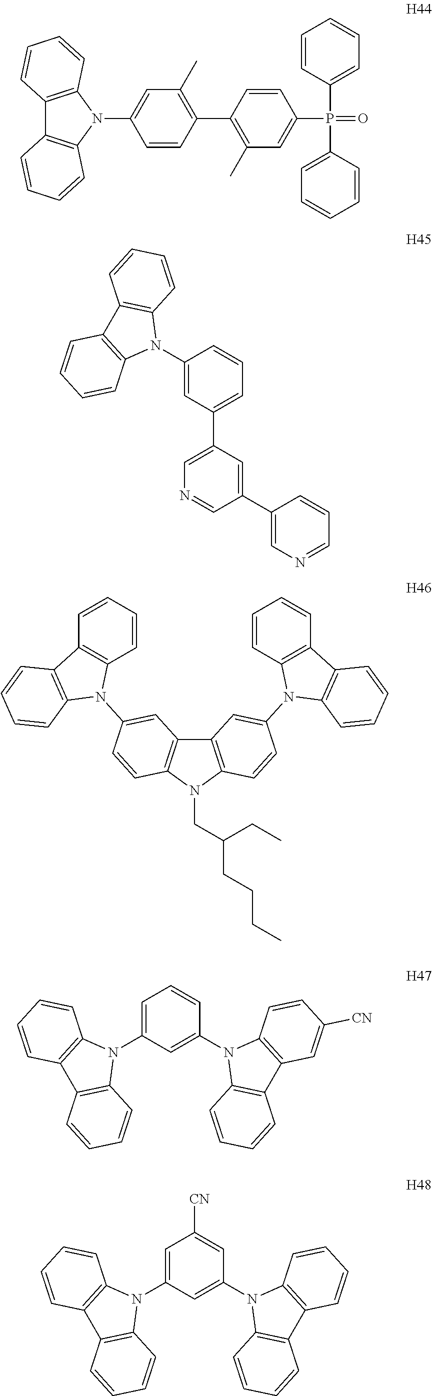

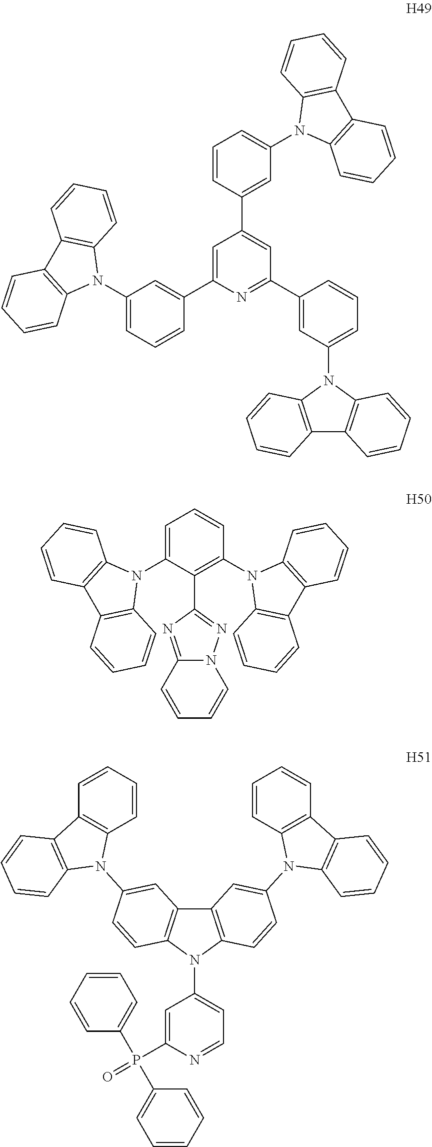 [SMILES: c1cc(-c2cc(-c3cccc(-n4c5ccccc5c5ccccc54)c3)nc(-c3cccc(-n4c5ccccc5c5ccccc54)c3)c2)cc(-n2c3ccccc3c3ccccc32)c1 ; c1cc(-n2c3ccccc3c3ccccc32)c(-c2nc3ccccn3n2)c(-n2c3ccccc3c3ccccc32)c1 ; O=P(c1ccccc1)(c1ccccc1)c1cc(-n2c3ccc(-n4c5ccccc5c5ccccc54)cc3c3cc(-n4c5ccccc5c5ccccc54)ccc32)ccn1]

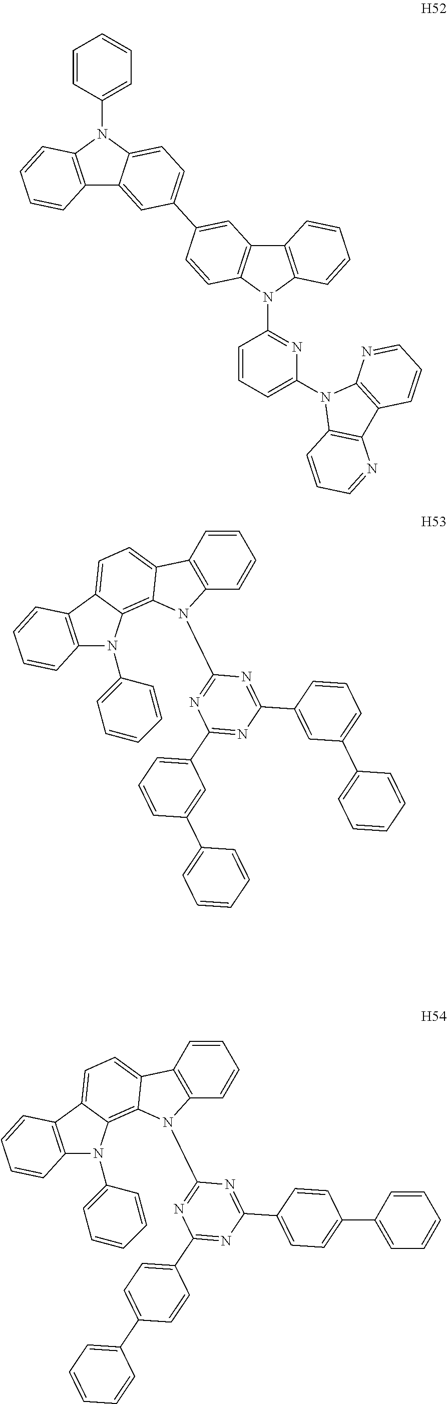 [SMILES: c1ccc(-n2c3ccccc3c3cc(-c4ccc5c(c4)c4ccccc4n5-c4cccc(-n5c6cccnc6c6cccnc65)n4)ccc32)cc1 ; c1ccc(-c2cccc(-c3nc(-c4cccc(-c5ccccc5)c4)nc(-n4c5ccccc5c5ccc6c7ccccc7n(-c7ccccc7)c6c54)n3)c2)cc1 ; c1ccc(-c2ccc(-c3nc(-c4ccc(-c5ccccc5)cc4)nc(-n4c5ccccc5c5ccc6c7ccccc7n(-c7ccccc7)c6c54)n3)cc2)cc1]

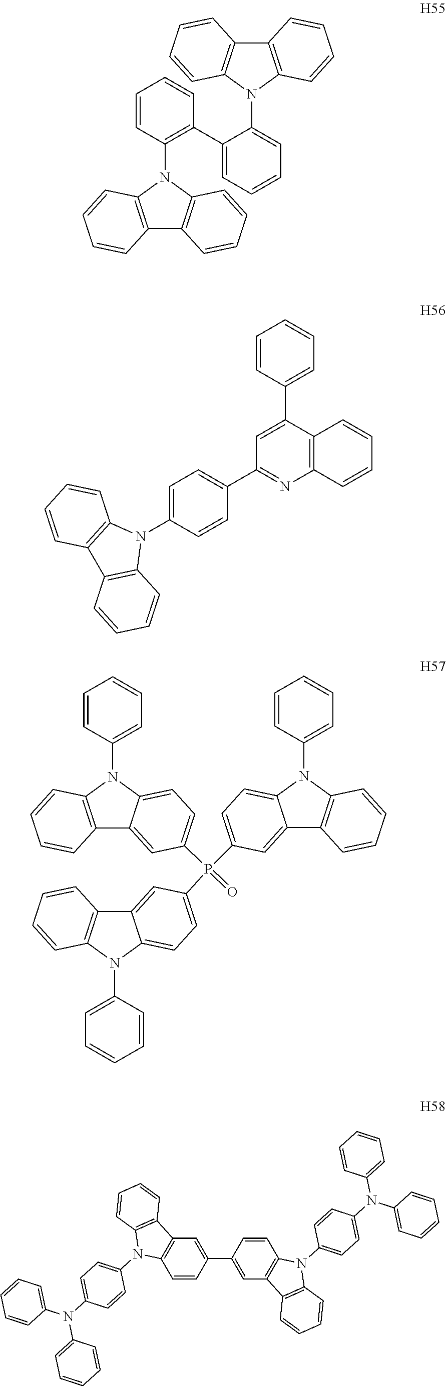

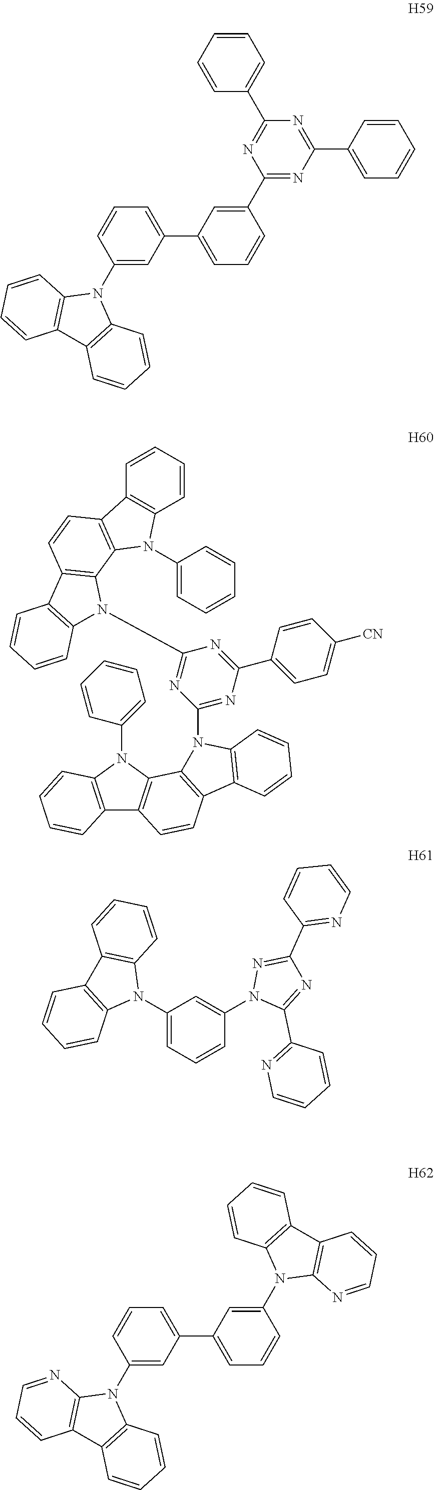 [SMILES: c1ccc(-c2nc(-c3ccccc3)nc(-c3cccc(-c4cccc(-n5c6ccccc6c6ccccc65)c4)c3)n2)cc1 ; N#Cc1ccc(-c2nc(-n3c4ccccc4c4ccc5c6ccccc6n(-c6ccccc6)c5c43)nc(-n3c4ccccc4c4ccc5c6ccccc6n(-c6ccccc6)c5c43)n2)cc1 ; c1ccc(-c2nc(-c3ccccn3)n(-c3cccc(-n4c5ccccc5c5ccccc54)c3)n2)nc1 ; c1cc(-c2cccc(-n3c4ccccc4c4cccnc43)c2)cc(-n2c3ccccc3c3cccnc32)c1]

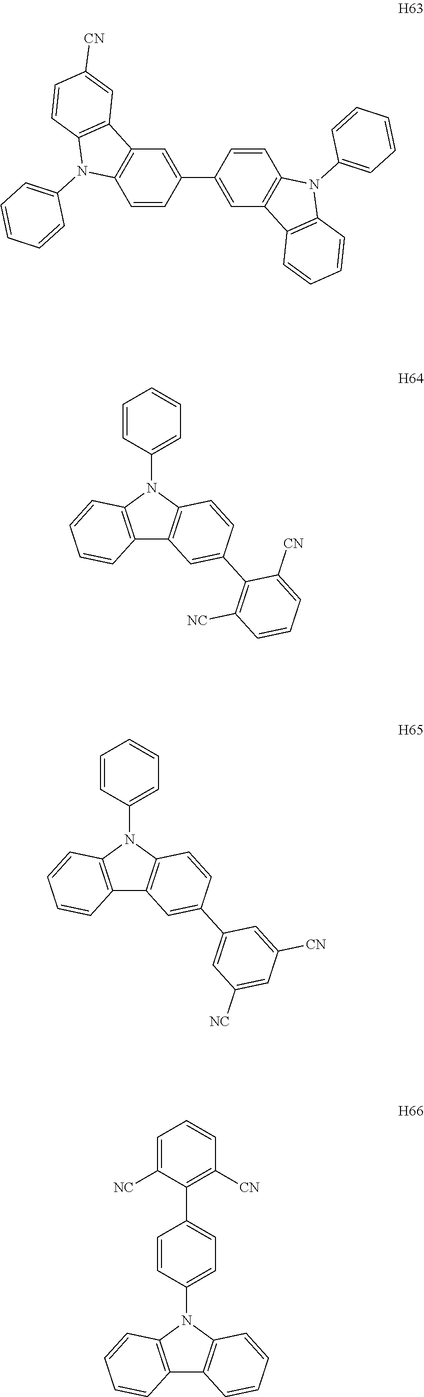 [SMILES: N#Cc1ccc2c(c1)c1cc(-c3ccc4c(c3)c3ccccc3n4-c3ccccc3)ccc1n2-c1ccccc1 ; N#Cc1cccc(C#N)c1-c1ccc2c(c1)c1ccccc1n2-c1ccccc1 ; N#Cc1cc(C#N)cc(-c2ccc3c(c2)c2ccccc2n3-c2ccccc2)c1 ; N#Cc1cccc(C#N)c1-c1ccc(-n2c3ccccc3c3ccccc32)cc1]

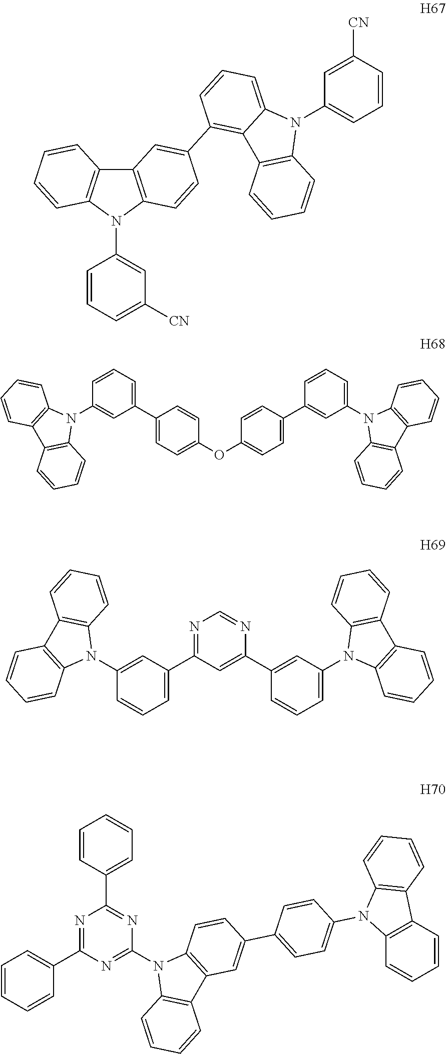 [SMILES: N#Cc1cccc(-n2c3ccccc3c3cc(-c4cccc5c4c4ccccc4n5-c4cccc(C#N)c4)ccc32)c1 ; c1cc(-c2ccc(Oc3ccc(-c4cccc(-n5c6ccccc6c6ccccc65)c4)cc3)cc2)cc(-n2c3ccccc3c3ccccc32)c1 ; c1cc(-c2cc(-c3cccc(-n4c5ccccc5c5ccccc54)c3)ncn2)cc(-n2c3ccccc3c3ccccc32)c1 ; c1ccc(-c2nc(-c3ccccc3)nc(-n3c4ccccc4c4cc(-c5ccc(-n6c7ccccc7c7ccccc76)cc5)ccc43)n2)cc1]

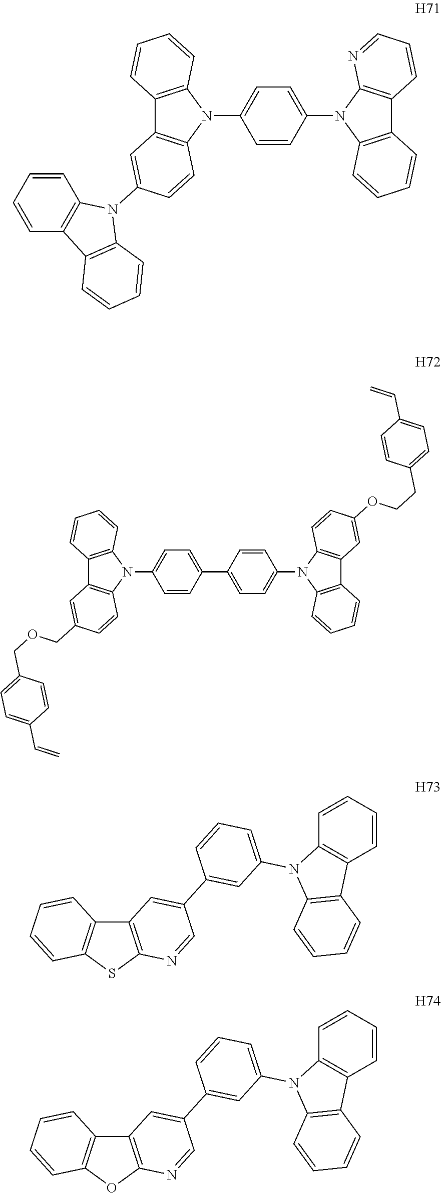 [SMILES: c1ccc2c(c1)c1ccccc1n2-c1ccc2c(c1)c1ccccc1n2-c1ccc(-n2c3ccccc3c3cccnc32)cc1 ; C=Cc1ccc(CCOc2ccc3c(c2)c2ccccc2n3-c2ccc(-c3ccc(-n4c5ccccc5c5cc(COCc6ccc(C=C)cc6)ccc54)cc3)cc2)cc1 ; c1cc(-c2cnc3sc4ccccc4c3c2)cc(-n2c3ccccc3c3ccccc32)c1 ; c1cc(-c2cnc3oc4ccccc4c3c2)cc(-n2c3ccccc3c3ccccc32)c1]

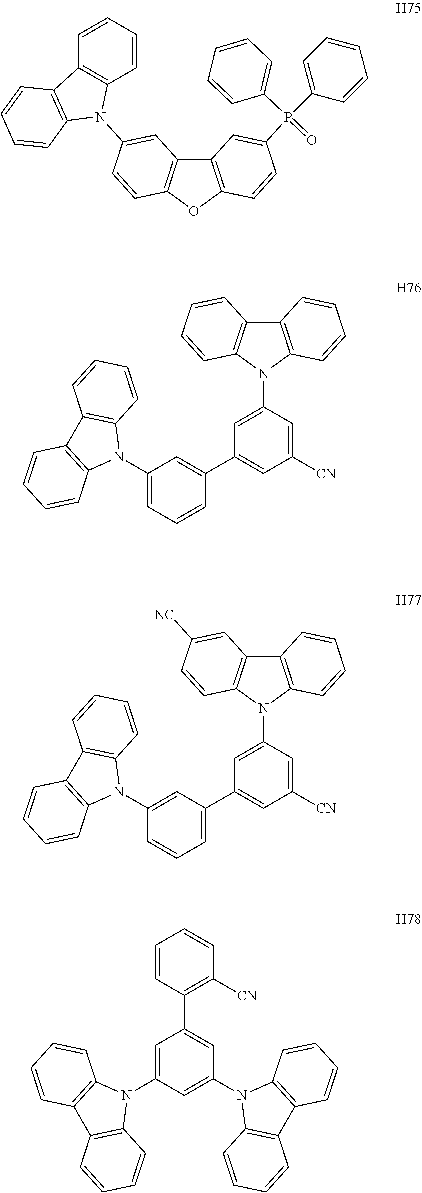 [SMILES: O=P(c1ccccc1)(c1ccccc1)c1ccc2oc3ccc(-n4c5ccccc5c5ccccc54)cc3c2c1 ; N#Cc1cc(-c2cccc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1ccccc1-c1cc(-n2c3ccccc3c3ccccc32)cc(-n2c3ccccc3c3ccccc32)c1]

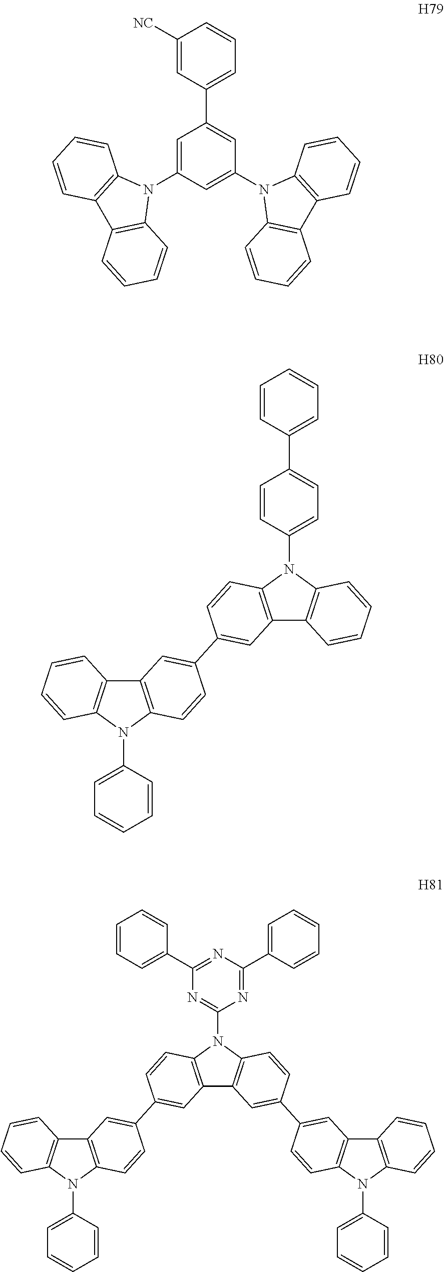 [SMILES: N#Cc1cccc(-c2cc(-n3c4ccccc4c4ccccc43)cc(-n3c4ccccc4c4ccccc43)c2)c1 ; c1ccc(-c2ccc(-n3c4ccccc4c4cc(-c5ccc6c(c5)c5ccccc5n6-c5ccccc5)ccc43)cc2)cc1 ; c1ccc(-c2nc(-c3ccccc3)nc(-n3c4ccc(-c5ccc6c(c5)c5ccccc5n6-c5ccccc5)cc4c4cc(-c5ccc6c(c5)c5ccccc5n6-c5ccccc5)ccc43)n2)cc1]

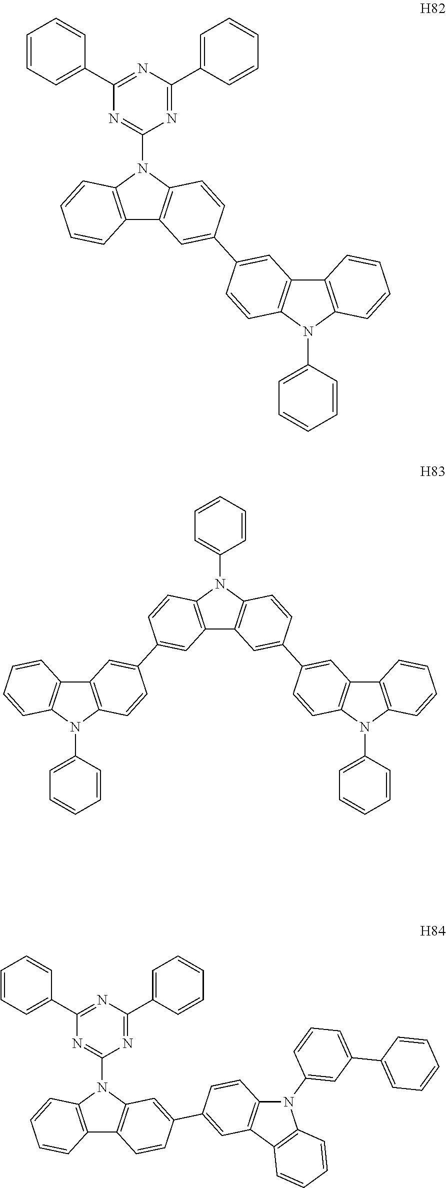 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-c2cccc(-n3c4ccccc4c4cc(-c5ccc6c7ccccc7n(-c7nc(-c8ccccc8)nc(-c8ccccc8)n7)c6c5)ccc43)c2)cc1]

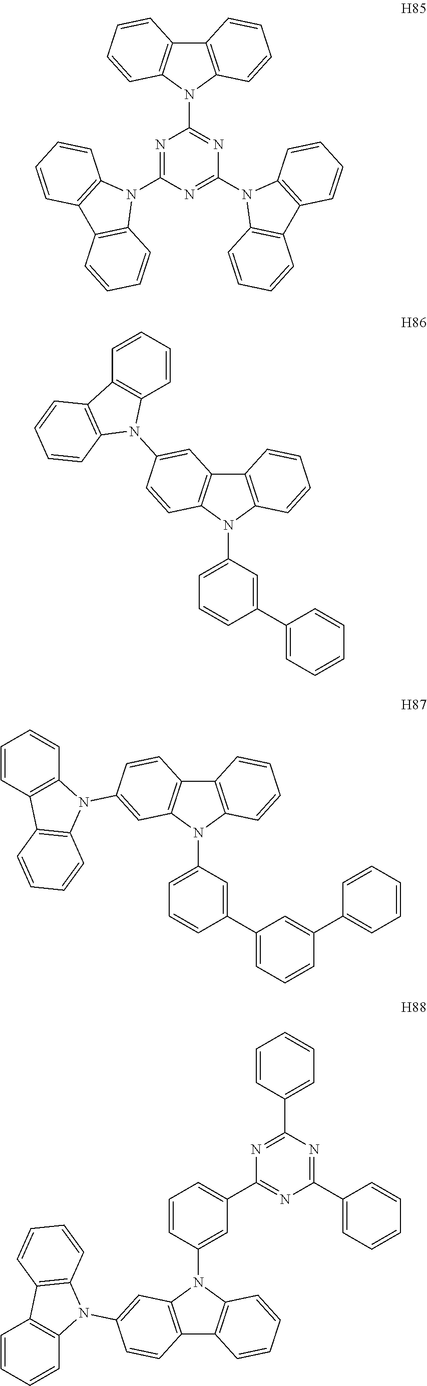 [SMILES: c1ccc2c(c1)c1ccccc1n2-c1nc(-n2c3ccccc3c3ccccc32)nc(-n2c3ccccc3c3ccccc32)n1 ; c1ccc(-c2cccc(-n3c4ccccc4c4cc(-n5c6ccccc6c6ccccc65)ccc43)c2)cc1 ; c1ccc(-c2cccc(-c3cccc(-n4c5ccccc5c5ccc(-n6c7ccccc7c7ccccc76)cc54)c3)c2)cc1 ; c1ccc(-c2nc(-c3ccccc3)nc(-c3cccc(-n4c5ccccc5c5ccc(-n6c7ccccc7c7ccccc76)cc54)c3)n2)cc1]

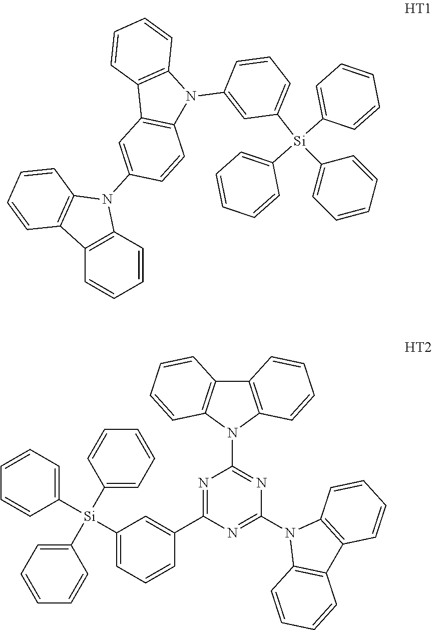 [SMILES: c1ccc([Si](c2ccccc2)(c2ccccc2)c2cccc(-n3c4ccccc4c4cc(-n5c6ccccc6c6ccccc65)ccc43)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1]

The material for an organic EL device according to an embodiment may include the compound of Formula (1), the phosphorescent complex, and the host material, wherein the host material may include the compound having the structure represented by Formula (5). The organic EL device according to another embodiment may include the compound of Formula (1) and the host material, wherein the host material may include the compound having the structure represented by Formula (5). The organic EL device according to another embodiment may include the compound of Formula (1), the phosphorescent complex, and the host material, wherein the host material may include at least two compounds having the structure represented by Formula (5). In this case, the compound having the structure represented by Formula (5) may include, for example, HT-1 and HT-2.

The compound having the triazine ring structure may be, but is not particularly limited to, a compound having a structure represented by Formula (6):

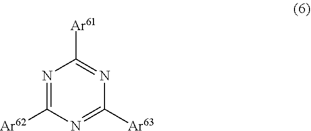

wherein, in Formula (6),
Ar⁶¹ to Ar⁶³ may each independently be a substituted or unsubstituted monovalent aromatic hydrocarbon group or a substituted or unsubstituted monovalent heterocyclic group.

The substituted or unsubstituted monovalent aromatic hydrocarbon group in Formula (6) is the same as described in connection with the group of (a7) of Formula (1). The substituted or unsubstituted monovalent heterocyclic group is the same as described in connection with the group of (a8) of Formula (1).

In Formula (6), substituents substituting the monovalent aromatic hydrocarbon group or the monovalent heterocyclic group may be, but are not particularly limited to, those mentioned above as the substituents substituting the groups of (a3) to (a9) in Formula (1). The substituent may also be a silyl group substituted with an unsubstituted monovalent aromatic hydrocarbon group. In addition, the unsubstituted monovalent aromatic hydrocarbon group is the same as described in connection with the unsubstituted group of (a7).

From among the compound having a triazine ring structure, a compound containing a silyl group (a compound having a triazine ring structure with a silyl group) may be preferred.

The compound having the triazine ring structure may be used in combination with the compound having the carbazole ring structure or the compound having the ring structure in which at least one ring-forming carbon atom of a carbazole ring is substituted with a nitrogen atom.

Hereinafter, the compound having the triazine structure, which is the host material according to an embodiment, will be described in detail. However, the disclosure is not limited to such examples.

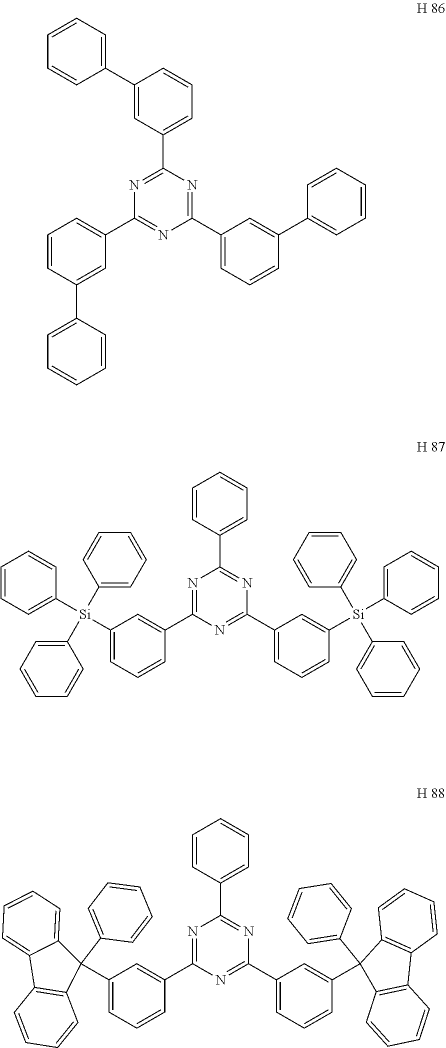 [SMILES: c1ccc(-c2cccc(-c3nc(-c4cccc(-c5ccccc5)c4)nc(-c4cccc(-c5ccccc5)c4)n3)c2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; c1ccc(-c2nc(-c3cccc(C4(c5ccccc5)c5ccccc5-c5ccccc54)c3)nc(-c3cccc(C4(c5ccccc5)c5ccccc5-c5ccccc54)c3)n2)cc1]

The material for an organic EL device according to an embodiment may include the compound of Formula (1), the phosphorescent complex, and the host material, wherein the host material may include the compound having the structure represented by Formula (6). Furthermore, the material for an organic EL device according to another embodiment may include the compound of Formula (1), the phosphorescent complex, and the host material, wherein the host material may include the compound having the structure represented by Formula (5) and the compound having the structure represented by Formula (6). The organic EL device according to another embodiment may include the compound of Formula (1) and the host material, wherein the host material may include the compound having the structure represented by Formula (6). The organic EL device according to another embodiment may include the compound of Formula (1) and the host material, wherein the host material may include the compound having the structure represented by Formula (5) and the compound having the structure represented by Formula (6).

An amount of the host material may be, but is not particularly limited to, 5 wt % or more based on the total weight of the material for an organic EL device (in particular, the material for an emission layer). In addition, the amount may be 10 wt % or more, for example, 20 wt % or more. When the amount is within the ranges above, an organic EL device with excellent luminescence color purity and high luminescence efficiency may be obtained. In addition, the amount of the host material may be, but is not particularly limited to, 99 wt % or less based on the total weight of the material for an organic EL device (in particular, the material for an emission layer). In addition, the amount may be 98 wt % or less, for example, 95 wt % or less. When the amount is within the ranges above, an organic EL device with excellent luminescence color purity and high luminescence efficiency may be obtained. An amount of the host material based on the total weight of an emission layer of an organic EL device to be described below may be the same as described above.

When the material for an organic EL device (in particular, the material for an emission layer) includes the host material, an amount of the host material may be 1,000 parts by weight or more based on 100 parts by weight of the compound of Formula (1). In addition, the amount may be 2,000 parts by weight or more, for example, 3,000 parts by weight or more, based on 100 parts by weight of the compound of Formula (1). When the amount is within the ranges above, an organic EL device with excellent luminescence color purity and high luminescence efficiency may be obtained. In addition, an amount of the host material may be, but is not particularly limited to, 200,000 parts by weight or less based on 100 parts by weight of the compound of Formula (1). In addition, the amount may be 150,000 parts by weight or less, for example, 100,000 parts by weight or less, based on 100 parts by weight of the compound of Formula (1). When the amount is within the ranges above, an organic EL device with excellent luminescence color purity and high luminescence efficiency may be obtained. An amount (parts by weight) of the host material based on 100 parts by weight of the compound of Formula (1) in an emission layer of an organic EL device to be described below may be the same as described above.

### Liquid Composition

Another aspect of the disclosure relates to a liquid composition including the compound of Formula (1), the material for an organic EL device, and a solvent.

The solvent is not particularly limited, and may be a solvent with a boiling point of about 100° C. to about 350° C. at atmospheric pressure (101.3 kPa, 1 atm). The boiling point of the solvent at atmospheric pressure may be about 150° C. to about 320° C., for example, about 180° C. to about 300° C. When the boiling point of the solvent at atmospheric pressure is within the ranges above, the processability or film-forming capability of a wet film forming method, in particular, an inkjet method, may be improved. The solvent with a boiling point of about 100° C. to about 350° C. at atmospheric pressure is not particularly limited, and a known solvent may be appropriately used. Hereinafter, the solvent with a boiling point of about 100° C. to about 350° C. at atmospheric pressure will be described in detail, but the disclosure is not limited thereto. Examples of a hydrocarbon-based solvent may include octane, nonane, decane, undecane, dodecane, and the like. Examples of an aromatic hydrocarbon-based solvent may include toluene, xylene, ethylbenzene, n-propylbenzene, iso-propylbenzene, mesitylene, n-butylbenzene, sec-butylbenzene, 1-phenylpentane, 2-phenylpentane, 3-phenylpentane, phenylcyclopentane, phenylcyclohexane, 2-ethylbiphenyl, 3-ethylbiphenyl, and the like. Examples of an ether-based solvent may include 1,4-dioxane, 1,2-diethoxyethane, diethyleneglycoldimethylether, diethyleneglycoldiethylether, anisole, ethoxybenzene, 3-methylanisole, m-dimethoxybenzene, and the like. Examples of a ketone-based solvent may include 2-hexanone, 3-hexanone, cyclohexanone, 2-heptanone, 3-heptanone, 4-heptanone, cycloheptanone, and the like. Examples of an ester-based solvent may include butylacetate, butylpropionate, butylbutyrate, propylenecarbonate, methylbenzoate, ethylbenzoate, 1-propylbenzoate, 1-butylbenzoate, and the like. Examples of a nitrile-based solvent may include benzonitrile, 3-methylbenzonitrile, and the like. Examples of an amide-based solvent may include dimethylformamide, dimethylacetamide, N-methylpyrrolidone, and the like. Such solvents may be used alone or in combination of two or more.

In an embodiment, the amount of the compound of Formula (1) and the material for the organic EL device in the liquid composition is not particularly limited.

In an embodiment, the liquid composition may be used as a coating liquid for forming an organic layer of an organic EL device. In addition, the liquid composition may be used as a coating liquid for forming an emission layer among coating liquids for forming an organic layer.

### Organic EL Device

Another aspect of the disclosure relates to an organic EL device including an emission layer including the compound of Formula (1). In the organic EL device, the emission layer may include the compound of Formula (1) and a phosphorescent complex, and the emission layer may further include a host material in addition to the compound of Formula (1) and the phosphorescent complex. The phosphorescent complex may be a platinum complex.

Another aspect of the disclosure relates to an organic EL device including the material for an organic EL device. In addition, the material for an organic EL device in the organic EL device may further include the host material. In addition, the phosphorescent complex included in the organic EL device may be a platinum complex.

The host material in the organic EL device may be the compound having the carbazole ring structure, the compound having a ring structure wherein at least one ring-forming carbon atom of the carbazole ring is substituted into a nitrogen atom, or the compound having the triazine ring structure. In addition, the host material included in the organic EL device may include the compound having the structure represented by Formula (5). In addition, the host material included in the organic EL device may include the compound having the structure represented by Formula (6). In addition, the host material included in the organic EL device may include the compound having the structure represented by Formula (5) and the compound having the structure represented by Formula (6).

In addition, the phosphorescent complex included in the organic EL device may be the compound having the structure represented by Formula (4).

The organic EL device according to an embodiment may include, but is not particularly limited to, a first electrode, a second electrode, and a single organic layer or a plurality of organic layers. The second electrode may be located on the first electrode.

Herein, when a portion of a layer, film, region, plate, or the like is said to be “on” or “above” another portion, this includes not only a case where the portion is “directly on” the other portion, but also a case where an intervening layer is present therebetween. In contrast, when a portion of a layer, film, region, plate, or the like is said to be “under” or “below” another portion, this includes not only a case where the portion is “directly under” the other portion, but also a case where an intervening layer is present therebetween. Herein, being located “on” includes not only being located on the top surface but also on the lower or bottom surface.

The organic EL device according to an embodiment may include a first electrode, a second electrode, and a single layer or a plurality of layers between the first electrode and the second electrode. In this regard, the layer or layers may include at least one organic layer, and at least one of the organic layer may include the compound of Formula (1) or the material for an organic EL device. The organic layer including the compound of Formula (1) or the material for an organic EL device may include an emission layer. Such an organic EL device may realize luminescence with high color purity.

As such, the emission layer may include at least one of the compound of Formula (1).

The emission layer may be a single layer including a single material or a single layer including a plurality of different materials. In addition, the emission layer may have a multi-layer structure with a plurality of layers including a plurality of different materials.

The emission layer is not particularly limited, and may include, for example, a host material and a dopant material. The compound of Formula (1) may be used as a host material or a dopant material, for example, as a dopant material.

In an embodiment, the organic EL device may include an emission layer, wherein the emission layer may include the compound of Formula (1) or the material for an organic EL device. The emission layer may include the material for an organic EL device. To provide the desired peak wavelength of the emission spectrum, luminescence color purity, luminescence efficiency, and device lifespan, the material for an organic EL device may include the host material in addition to the compound of Formula (1). In the same point of view, the material for an organic EL device may include the phosphorescent complex and the host material in addition to the compound of Formula (1). In addition, an amount or amount ratio of each of the compound of Formula (1), the phosphorescent complex, and the host material in the emission layer may be in the same range as the amount or amount ratio of the material for an organic EL device.

A thickness of the emission layer may be, but is not particularly limited to, about 1 nm to about 100 nm, for example, about 10 nm to about 50 nm.

Examples of the film forming method of the emission layer may include, but are not particularly limited to, known film forming methods such as vacuum deposition, spin coating, Langmuir-Blodgett (LB) deposition, ink-jet printing, laser-printing, and laser-induced thermal imaging (LITI).

An emission wavelength of the organic EL device is not particularly limited. The emission wavelength of the organic EL device may be, for example, in the same range as the peak wavelength of emission in PL of the compound of Formula (1) according to the disclosure. In specifications of currently commercialized products, in the case of blue luminescence, the organic EL device may emit light with a peak in a wavelength region of about 445 nm to about 470 nm, for example, about 450 nm to about 470 nm, or for example, about 450 nm to about 465 nm.

In addition, a FWHM of a peak of an emission spectrum of the organic EL device may be smaller. In addition, the FWHM of the peak of the emission spectrum may be 30 nm or less, for example, 25 nm or less. In addition, the FWHM of the peak of the emission spectrum may be 20 nm or less (the lower limit is greater than 0 nm).

Hereinafter, a case where the organic EL device according to an embodiment further includes an organic layer, in addition to the emission layer, will be described in detail with reference to the accompanying drawings. In the description of the drawings, the same components will be denoted by the same reference numerals, and redundant descriptions thereof will be omitted. In addition, dimensional ratios in the drawings may be exaggerated for convenience of description, and thus may differ from actual ratios.

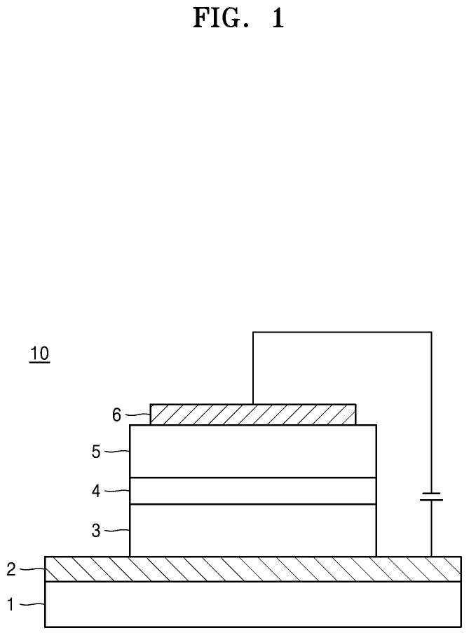

FIGS. 1 to 3 are each a schematic cross-sectional view illustrating an organic EL device according to an embodiment. However, the structure of the organic EL device according to the disclosure is not limited to those shown in FIGS. 1 to 3.

FIG. 1 is a schematic cross-sectional view illustrating an organic EL device according to an embodiment. The organic EL device 10 according to an embodiment may include a substrate 1, a first electrode 2, a hole transport region 3, an emission layer 4, an electron transport region 5, and a second electrode 6, which are sequentially stacked in this stated order.

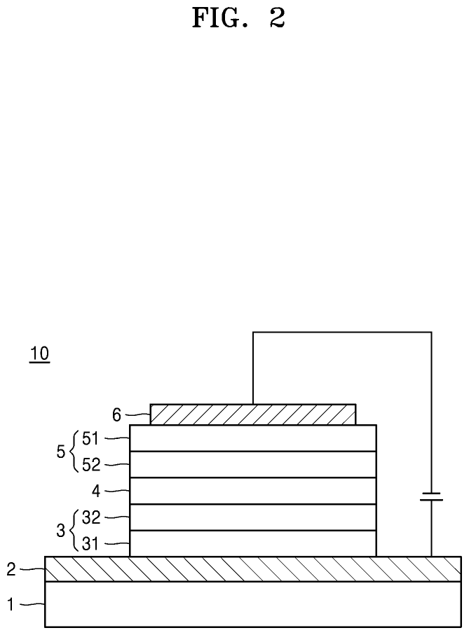

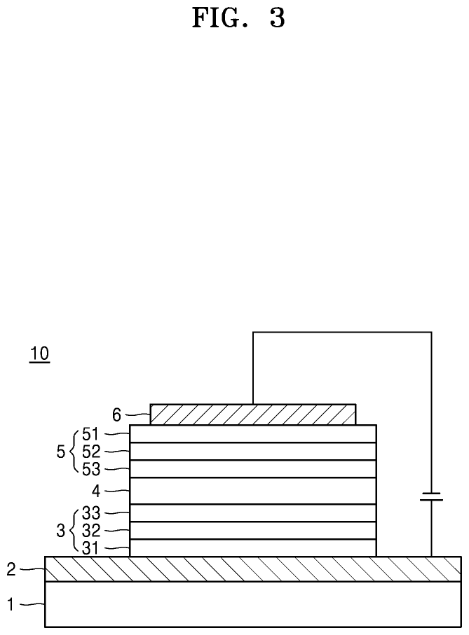

FIG. 2 is a schematic cross-sectional view illustrating an organic EL device according to another embodiment. The organic EL device 10 according to an embodiment may include a substrate 1, a first electrode 2, a hole transport region 3, an emission layer 4, an electron transport region 5, and a second electrode 6, which are sequentially stacked in this stated order. In FIG. 2, the hole transport region 3 may include a hole injection layer 31 and a hole transport layer 32, which are sequentially stacked in this stated order. In addition, in FIG. 3, the electron transport region 5 may include an electron transport layer 52 and an electron injection layer 51, which are sequentially stacked in this stated order.

FIG. 3 is a schematic cross-sectional view illustrating an organic EL device according to another embodiment. The organic EL device 10 according to an embodiment may include a substrate 1, a first electrode 2, a hole transport region 3, an emission layer 4, an electron transport region 5, and a second electrode 6, which are sequentially stacked in this stated order. In FIG. 3, the hole transport region 3 may include a hole injection layer 31, a hole transport layer 32, and an electron blocking layer 33, which are sequentially stacked in this stated order. In addition, in FIG. 3, the electron transport region 5 may include a hole blocking layer 53, an electron transport layer 52, and an electron injection layer 51, which are sequentially stacked in this stated order.

Hereinafter, the substrate 1, each region, and each layer will be described in detail.

### Substrate 1

The organic EL device 10 may have a substrate 1. As the substrate 1, any substrate used in a general organic EL device may be used. For example, the substrate 1 may be a glass substrate, a semiconductor substrate such as a silicon substrate, or a transparent plastic substrate.

### First Electrode 2

The first electrode 2 may have conductivity. In the organic EL device 10 according to an embodiment, the first electrode 2 may be an anode. In addition, the first electrode 2 may be a pixel electrode. In addition, the first electrode 2 may be a reflective electrode, a semi-reflective electrode, or a transmissive electrode.

A material for forming the first electrode 2 is not particularly limited, and may be, for example, a metal, a metal alloy, or a conductive compound. When the first electrode 2 is a transmissive electrode, the first electrode 2 may include a transparent metal oxide, for example, indium tin oxide (“ITO”), indium zinc oxide (“IZO”), zinc oxide (ZnO), indium tin zinc oxide (“ITZO”), or the like. When the first electrode 2 is a semi-transmissive electrode or a reflective electrode, the first electrode 2 may include Ag, Mg, Cu, Al, Pt, Pd, Au, Ni, Nd, Ir, Cr, Li, Ca, LiF/Ca, LiF/AI, Mo, Ti, or a compound or mixture thereof (e.g., a mixture of Ag and Mg).

The first electrode 2 may be a single layer including a single material or a single layer including a plurality of different materials. In addition, the first electrode 2 may have a multi-layer structure with a plurality of layers including a plurality of different materials.

A thickness of the first electrode 2 may be, but is not particularly limited to, about 10 nm to about 1,000 nm, for example, about 50 nm to about 300 nm.

### Hole Transport Region 3

The hole transport region 3 may be provided on the first electrode 2. The hole transport region 3 may include at least one of the hole injection layer 31, the hole transport layer 32, a hole buffer layer (not shown), and the electron blocking layer 33.

The hole transport region 3 may be a single layer including a single material or a single layer including a plurality of different materials. In addition, the hole transport region 3 may have a multi-layer structure with a plurality of layers including a plurality of different materials.

For example, the hole transport region 3 may have a single-layer structure including the hole injection layer 31 or the hole transport layer 32. In addition, for example, the hole transport region 3 may have a single-layer structure including a hole injection material and a hole transport material. In addition, for example, the hole transport region 3 may have a hole injection layer 31/hole transport layer 32 structure, wherein constituting layers are sequentially stacked from the first electrode 2. In addition, for example, the hole transport region 3 may have a hole injection layer 31/hole transport layer 32/hole buffer layer (not shown) structure. In addition, for example, the hole transport region 3 may have a hole injection layer 31/hole buffer layer (not shown) structure, wherein constituting layers are sequentially stacked from the first electrode 2. In addition, for example, the hole transport region 3 may have a hole transport layer 32/hole buffer layer (not shown) structure, wherein constituting layers are sequentially stacked from the first electrode 2. In addition, for example, the hole transport region 3 may have a hole injection layer 31/hole transport layer 32/electron blocking layer 33 structure, wherein constituting layers are sequentially stacked from the first electrode 2. However, the structure of the hole transport region 3 is not limited to the examples above.

The hole injection layer 31 or other layers constituting the hole transport region 3 are not particularly limited, and may include, for example, a known hole injection material. Examples of the hole injection material may include a phthalocyanin compound such as copper phthalocyanin, N, N′-diphenyl-N, N′-bis-[4-phenyl-m-toly-amino)-phenyl]-biphenyl-4,4′-diamine (“DNTPD”), 4,4′,4″-tris(3-methylphenylphenylamino)triphenylamine (“m-MTDATA”), 4,4′,4″-tris(N,N-diphenylamino)triphenylamine (“TDATA”), 4,4′,4″-tris {N,-(2-naphthyl)-N-phenylamino}-triphenylamine (“2-TNATA”), poly(3,4-ethylenedioxythiophene)/poly(4-styrenesulfonate) (“PEDOT” “PSS”), polyaniline/dodecylbenzenesulfonic acid (“PANI”/“DBSA”), polyaniline/camphor sulfonic acid (PANI/“CSA”), polyaniline/poly(4-styrenesulfonate) (PANI/PSS), N, N′-di(naphthalene-1-yl)-N,N′-diphenyl-benzidine (“NPB”), polyetherketone including triphenylamine (“TPAPEK”), 4-isopropyl-4′-methyldiphenyliodonium tetrakis(pentafluorophenyl)borate, dipyrazino[2,3-f: 2′,3′-h]quinoxaline-2,3,6,7,10,11-hexacarbonitrile (“HAT-CN”), 1,3,4,5,7,8-hexafluorotetracyano-2,6-naphthoquinodimethane (“F6-TCNNQ”), and the like.

In addition, the hole transport layer 32 or other layers constituting the hole transport region 3 are not particularly limited, and may include, for example, a known hole transport material. Examples of the hole transport material may include N-phenylcarbazole, a carbazole-based derivative such as polyvinyl carbazole, a fluorene-based derivative, N,N′-bis(3-methylphenyl)-N, N′-diphenyl-[1,1-biphenyl]-4,4′-diamine (“TPD”), a triphenylamine-based derivative such as 4,4′,4″-tris(N-carbazolyl)triphenylamine (“TCTA”), N,N′-di(naphthalene-1-yl)-N,N′-diphenyl-benzidine (NPB), 4,4′-cyclohexylidenebis[N,N-bis(4-methylphenyl)benzeneamine] (“TAPC”), 4,4′-bis[N,N′-(3-tolyl amino]-3,3′-dimethylbiphenyl (“HMTPD”), 1,3-bis(N-carbazolyl)benzene (“mCP”), Compound HTM1, Compound HTM2, Compound HT3, and the like.

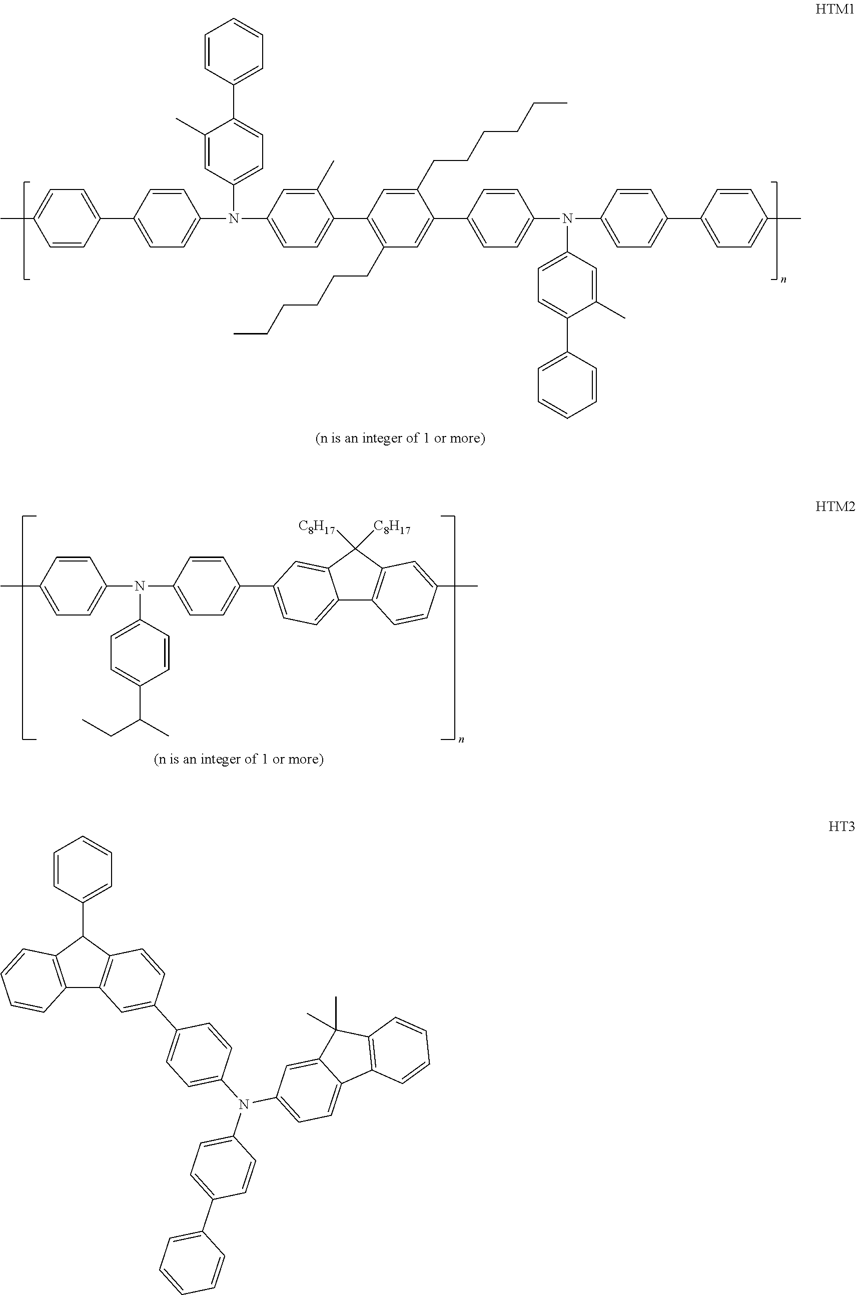

The hole transport region 3 may further include a charge generation material, in addition to the hole injection material or the hole transport material, to improve conductivity. The charge generation material may be homogeneously or non-homogeneously dispersed in the hole transport region 3 or each layer thereof. The charge generation material is not particularly limited, and may be, for example, a known charge generation material. The charge generation material may be, for example, a p-dopant. Examples of the p-dopant may include a quinone derivative such as tetracyanoquinonedimethane (“TCNQ”) or 2,3,5,6-tetrafluoro-tetracyanoquinodimethane (“F4-TCNQ”), a metal oxide such as tungsten oxide or molybdenum oxide, a cyano group-containing compound, and the like.

The buffer layer (not shown) may compensate for an optical resonance distance according to a wavelength of light emitted from the emission layer 4 to improve luminescence efficiency. Materials included in the hole buffer layer (not shown) are not particularly limited, and materials used in a known hole buffer layer may be used. For example, the compounds that may be included in the hole transport region 3 may be used.

The electron blocking layer 33 may prevent injection of electrons from the electron transport region 5 to the hole transport region 3. Materials included in the electron blocking layer 33 are not particularly limited, and materials used in a known electron blocking layer may be used. For example, the host material, such as Compounds H55, H86, and H87, included in the emission layer (the material for an organic EL device) may be used.

A thickness of the hole transport region 3 may be, but is not particularly limited to, about 1 nm to about 1,000 nm, for example, about 10 nm to about 500 nm. In addition, regarding each layer constituting the hole transport region 3, a thickness of the hole injection layer 31 may be, but is not particularly limited to, about 3 nm to about 200 nm. A thickness of the hole transport layer 32 may be, but is not particularly limited to, about 3 nm to about 200 nm. A thickness of the electron blocking layer 33 may be, but is not particularly limited to, about 1 nm to about 100 nm. In addition, a thickness of the hole buffer layer (not shown) is not particularly limited, as long as the hole buffer layer functions as a hole buffer layer and does not interfere with functions of an organic EL device. When the thickness of the hole transport region 3, the hole injection layer 31, the hole transport layer 32, or the electron blocking layer 33 is within the ranges above, excellent hole transport characteristics may be obtained while suppressing a substantial increase in driving voltage.

Examples of the film forming method of the hole transport region 3 or each layer thereof may include, but are not particularly limited to, known film forming methods such as vacuum deposition, spin coating, LB deposition, ink-jet printing, laser-printing, and LITI.

### Emission Layer 4

The emission layer 4 may be located on the hole transport region 3. Details of the emission layer 4 may be the same as described above.

### Electron Transport Region 5

The electron transport region 5 may be located on the emission layer 4. The electron transport region 5 may include at least one of the electron injection layer 51, the electron transport layer 52, and the hole blocking layer 53, but embodiments are not limited thereto.

The electron transport region 5 may be a single layer including a single material or a single layer including a plurality of different materials. In addition, the electron transport region 5 may have a multi-layer structure with a plurality of layers including a plurality of different materials. For example, the electron transport region 5 may have a single-layer structure including the electron injection layer 51 or the electron transport layer 52. In addition, for example, the electron transport region 5 may have a single-layer structure including an electron injection material and an electron transport material. In addition, for example, the electron transport region 5 may have an electron transport layer 52/electron injection layer 51 structure, wherein constituting layers are sequentially stacked from the emission layer 4. In addition, for example, the electron transport region 5 may have a hole blocking layer 53/electron transport layer 52/electron injection layer 51 structure, wherein constituting layers are sequentially stacked from the emission layer 4. However, the structure of the electron transport region 5 is not limited to the examples above.

The electron injection layer 51 or other layers constituting the electron transport region 5 are not particularly limited, and may include, for example, a known electron injection material. Examples of the electron injection material may include a lanthanide metal (such as Yb), LiF, lithium quinolate (“LiQ”), Li₂O, BaO, NaCl, CsF, or a metal halide such as RbCl. The electron injection layer 51 is not particularly limited, and may include, for example, the electron transport material and an insulating organometallic salt. The organometallic salt is not particularly limited, and may be, for example, a material with an energy band gap of 4 electronvolts (eV) or greater. The organometallic salt may be, for example, an acetate metallic salt, a benzoate metallic salt, an acetoacetate metallic salt, an acetylacetonate metallic salt, or a stearate metallic salt.

The electron transport layer 52 or other layers constituting the electron transport region 5 are not particularly limited, and may include, for example, a known electron transport material. Examples of the electron transport material may include an anthracene-based compound, tris(8-hydroxyquinolinolato)aluminum) (“Alq₃”), 1,3,5-tri[(3-pyridyl)-pen-3-yl]benzene, 2,4,6-tris(3′-pyridine-3-yl)biphenyl-3-yl)-1,3,5-triazine, 2-(4-(N-phenylbenzimidazolyl-1-ylphenyl)-9,10-dinaphthylanthracene, 1,3,5-tri(1-phenyl-1H-benzo[d]imidazol-2-yl)phenyl (“TPBi”), 2,9-dimethyl-4,7-diphenyl-1,10-phenanthroline (“BCP”), 4,7-diphenyl-1,10-phenanthroline (“Bphen”), 3-(4-biphenyl)-4-phenyl-5-tert-butylphenyl-1,2,4-triazole (“TAZ”), 4-(naphthalen-1-yl)-3,5-diphenyl-4H-1,2,4-triazole (“NTAZ”), 2-(4-biphenyl)-5-(4-tert-butylphenyl)-1,3,4-oxadiazole (“tBu-PBD”), bis(2-methyl-8-quinolinolato-N1,O8)-(1,1′-biphenyl-4-olato)aluminum (“BAlq”), beryllium bis(benzoquinoline-10-olato) (“Bebq₂”), 9,10-di(naphthalen-2-yl)anthracene (“ADN”), lithium quinolate (LiQ), Compound ET1, and the like. In addition, “TRE314” (product of Toray Co., Ltd., electron transport material) or the like may be used.

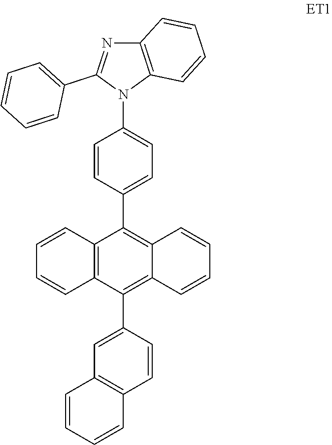 [SMILES: c1ccc(-c2nc3ccccc3n2-c2ccc(-c3c4ccccc4c(-c4ccc5ccccc5c4)c4ccccc34)cc2)cc1]

The hole blocking layer 53 may prevent injection of holes from the hole transport region 3 to the electron transport region 5. Materials included in the hole blocking layer 53 are not particularly limited, and materials used in a known hole blocking layer may be used. The hole blocking layer 53 may include, for example, a known hole blocking material. Examples of the hole blocking material may include 2,9-dimethyl-4,7-diphenyl-1,10-phenanthroline (BCP), 4,7-diphenyl-1,10-phenanthroline (BPhen), and the like. In addition, for example, the host material, such as Compounds H77 and H87, included in the emission layer (the material for an organic EL device) may be used.

A thickness of the electron transport region 5 may be, but is not particularly limited to, about 0.1 nm to about 200 nm, for example, about 30 nm to about 150 nm. In addition, regarding each layer constituting the electron transport region 5, a thickness of the electron transport layer 52 may be, but is not particularly limited to, about 10 nm to about 100 nm, for example, about 15 nm to about 50 nm. A thickness of the hole blocking layer 53 may be, but is not particularly limited to, about 1 nm to about 100 nm, for example, about 5 nm to about 30 nm. A thickness of the electron injection layer 51 may be, but is not particularly limited to, about 0.1 nm to about 10 nm, for example, about 0.3 nm to about 9 nm. When the thickness of the electron injection layer 51 is within the ranges above, excellent electron injection characteristics may be obtained while suppressing a substantial increase in driving voltage. In addition, when the thickness of the electron transport region 5, the electron injection layer 51, the electron transport layer 52, or the hole blocking layer 53 is within the ranges above, excellent hole transport characteristics may be obtained while suppressing a substantial increase in driving voltage.

Examples of the film forming method of the electron transport region 5 or each layer thereof may include, but are not particularly limited to, known film forming methods such as vacuum deposition, spin coating, LB deposition, ink-jet printing, laser-printing, and LITI.

The second electrode 6 may be located on the electron transport region 5. The second electrode 6 may have conductivity. In the organic EL device 10 according to an embodiment, the second electrode 6 may be a common electrode or a cathode. In addition, the second electrode 6 may be a reflective electrode, a semi-reflective electrode, or a transmissive electrode.

A material for forming the second electrode 6 is not particularly limited, and may be, for example, a metal, a metal alloy, or a conductive compound. When the second electrode 6 is a reflective electrode, the second electrode 6 may include a transparent metal oxide, for example, ITO, IZO, ZnO, ITZO, or the like. When the second electrode 6 is a semi-transmissive electrode or a reflective electrode, the second electrode 6 may include Ag, Mg, Cu, Al, Pt, Pd, Au, Ni, Nd, Ir, Cr, Li, Ca, LiF/Ca, LiF/AI, Mo, Ti, or a compound or mixture thereof (e.g., a mixture of Ag and Mg).

The second electrode 6 may be a single layer including a single material or a single layer including a plurality of different materials. In addition, the second electrode 6 may have a multi-layer structure with a plurality of layers including a plurality of different materials.

A thickness of the second electrode 6 may be, but is not particularly limited to, about 10 nm to about 1,000 nm.

The second electrode 6 may be connected to an auxiliary electrode (not shown). By connecting the second electrode 6 to the auxiliary electrode, the resistance of the second electrode 6 may be reduced.

In addition, a capping layer (not shown) may be further located on the second electrode 6. Examples of the capping layer (not shown) may include, but are not particularly limited to, “α-NPD”, NPB, TPD, m-MTDATA, Alq₃, “CuPc”, N4,N4, N4′, N4′-tetra(biphenyl-4-yl) biphenyl-4,4′-diamine (“TPD15”), 4,4′,4″-tri-9-carbazolyltriphenylamine (TCTA), N, N′-bis(naphthalen-1-yl), and the like.

Materials constituting each layer and each electrode may be used alone or in combination of two or more.

In the organic EL device 10 of FIGS. 1 to 3, the compound of Formula (1) or the material for an organic EL device may be included in the emission layer 4, but may also be included in organic layers other than the emission layer 4. In addition, the compound of Formula (1) or the material for an organic EL device may be included in the emission layer 4 and the organic layers other than the emission layer 4.

In the organic EL device 10 of FIGS. 1 to 3, since a voltage is applied to each of the first electrode 2 and the second electrode 6, holes provided from the first electrode 2 may move toward the emission layer 4 through the hole transport region 3, and electrons provided from the second electrode 6 may move toward the emission layer 4 through the electron transport region 5. The holes and the electrons may recombine in the emission layer 4 to produce excitons, and these excitons may transition from an excited state to a ground state to thereby generate light.

### EXAMPLES

Hereinafter, the disclosure will be described in more detail with reference to the examples and comparative examples, but the technical scope of the disclosure is not limited to the following examples.

### Synthesis of Compound

Compound 1 was synthesized as described below to be used in the manufacture of an organic EL device. Compound C was also prepared for comparison and to be used in the manufacture of a comparative organic EL device.

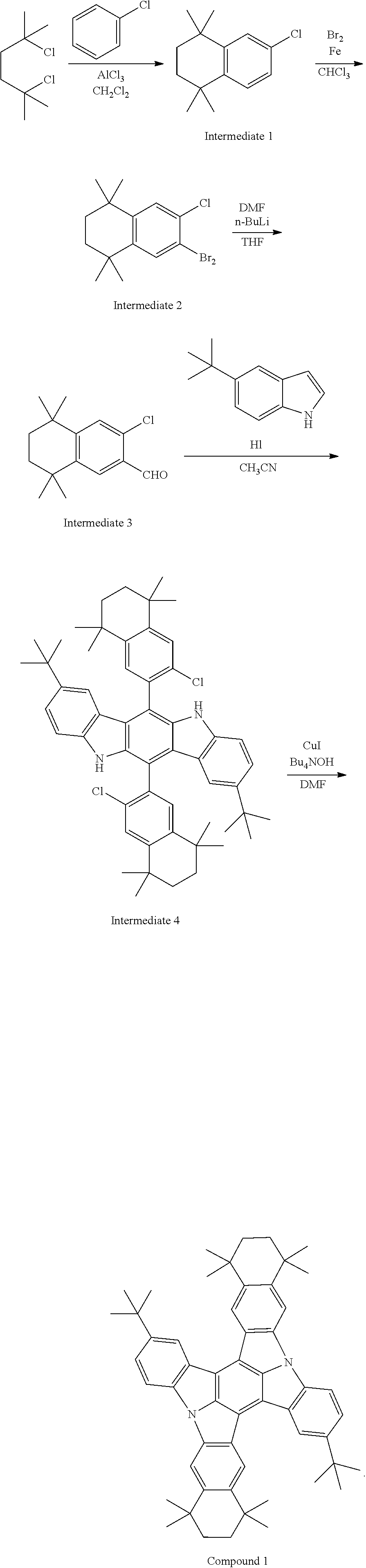

### Synthesis Example 1

### Synthesis of Intermediate 1

Chlorobenzene [60 milliliters (mL), 0.6 moles (mol)) and dichloromethane (500 mL) were added to a 1 (liter) L three-neck flask under a nitrogen atmosphere, and the solution was then cooled to −30° C. After adding aluminum chloride (1.6 grams (g), 0.012 mol) thereto, a dichloromethane solution (120 mL) of 2,4-dichloro-2,4-dimethylhexane (34 g, 0.19 mol) was added dropwise over 30 minutes. The mixture was stirred for 30 minutes, and the temperature was raised to −20° C. After 1 hour, 100 mL of 2 moles per liter (M) aqueous hydrochloric acid solution was added. After separation of the aqueous layer, extraction was performed thereon with dichloromethane (50 mL×2). The combined organic layers were washed with a saturated aqueous ammonium chloride solution (50 mL) and dried with anhydrous sodium sulfate. After filtration, the filtrate was concentrated under reduced pressure to provide Intermediate 1 as a colorless liquid (42 g, Yield: 100%).

### Synthesis of Intermediate 2

Intermediate 1 (42 g, 0.19 mol) and dichloromethane (300 mL) were added to a 1 L three-neck flask under a nitrogen atmosphere. After adding iron (0.3 g) thereto, a dichloromethane solution (60 mL) of bromine (30 g, 0.19 mol) was added dropwise to the mixture over 45 minutes. The mixture was stirred at room temperature for 2 hours, and a saturated aqueous sodium hydrogen carbonate solution (50 mL) was added thereto. After separation of the aqueous layer, extraction was performed thereon with dichloromethane (50 mL×2). The combined organic layers were washed with a saturated aqueous sodium hydrogen carbonate solution (50 mL) and then a saturated saline water (50 mL) and dried with anhydrous sodium sulfate. After filtration, the filtrate was concentrated under reduced pressure. The obtained residue was reprecipitated with dichloromethane-methanol to provide Intermediate 2 as a white crystalline solid (48 g, Yield: 83%).

### Synthesis of Intermediate 3

Intermediate 2 (15 g, 50 mol) and THF (500 mL) were combined in a 1 L three-neck flask under a nitrogen atmosphere. After cooling the mixture to −78° C., DMF (10 mL, 0.13 mol) and n-butyllithium (1.6 M hexane solution, 78 mL) were added dropwise over 30 minutes. Then, the solution temperature was slowly raised to room temperature. After the mixture was stirred for 12 hours at room temperature, water (100 mL) and 1 M aqueous hydrochloric acid solution (50 mL) were added thereto. After separation of the aqueous layer, extraction was performed thereon with ethyl acetate (100 mL×2). The combined organic layers were washed with a saturated saline water (50 mL) and then a saturated aqueous sodium hydrogen carbonate solution (30 mL). After the organic layer was dried with anhydrous sodium sulfate, a desiccant was removed by filtration, and the filtrate was concentrated under reduced pressure. The obtained residue was reprecipitated with dichloromethane-methanol to provide Intermediate 3 as a white powder (11.7 g, Yield: 90%).

### Synthesis of Intermediate 4

5-tert-butyl-1H-indole [1.3 g, 7.6 millimoles (mmol)), Intermediate 3 (1.9 g, 7.6 mmol), and 38 mL of acetonitrile were added to a 100 mL three-neck flask under a nitrogen atmosphere. After raising the temperature to 80° C., 57% hydroiodic acid (0.2 mL, 1.5 mmol) was added thereto, and the mixture was stirred for 2 hours. After cooling to room temperature, the precipitated solid was filtered and washed with cooled acetonitrile to provide Intermediate 3 as a light orange powder (1.8 g, Yield: 57%).

### Synthesis of Compound 1

Intermediate 4 (2.3 g, 2.5 mmol), tetrabutylammoniumhydroxide (37% methanol solution) (10.5 mL, 12.4 mmol), copper (I) iodide (2.4 g, 12.4 mmol), and 25 mL of dimethylacetamide were added to a 100 mL three-neck flask under a nitrogen atmosphere and then heated while stirring at 140° C. Tetrabutylammoniumhydroxide (37% methanol solution) (10.5 mL, 12.4 mmol) and copper (I) iodide (2.4 g, 12.4 mmol) were added thereto every 8 hours and then the reaction mixture was stirred for 24 hours. After cooling to room temperature, the precipitated solid was filtered. The filtered solid, 200 mL of methanol, and 50 mL of ethylenediamine were combined in a 500 mL conical flask and stirred, and the solid was collected by filtration to provide Compound 1 as a yellow solid (1.2 g, Yield: 66%, LC-MS: 737([M+H]⁺).

### Compound Data of Compound 1 (Identification Data)

The structure of the obtained Compound 1 was characterized by nuclear magnetic resonance (1H-NMR):

1H-NMR (300 MHZ, CD₂Cl₂) δ1.45 (s, 12H), 1.46 (s, 12H), 1.53 (s, 18H), 1.82 (s, 8H), 7.67 (dd, 2H, J=8.4 Hz, 1.8 Hz), 7.92 (s, 2H), 7.98 (d, 2H, J=8.4 Hz), 8.48 (s, 2H), 8.57 (d, 2H, J=1.8 Hz).

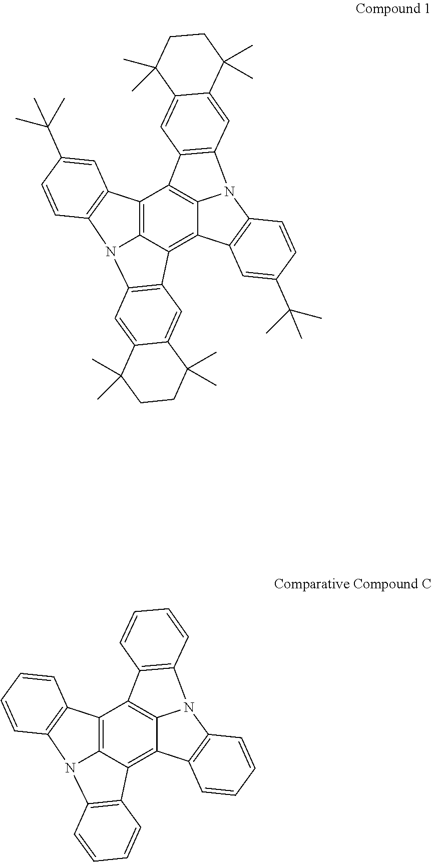 [SMILES: CC(C)(C)c1ccc2c(c1)c1c3c4cc5c(cc4n4c6ccc(C(C)(C)C)cc6c(c6c7cc8c(cc7n2c16)C(C)(C)CCC8(C)C)c34)C(C)(C)CCC5(C)C ; c1ccc2c(c1)c1c3c4ccccc4n4c5ccccc5c(c5c6ccccc6n2c15)c34]

### Evaluation of Properties

Emission characteristics simulation was conducted with respect to Compound 1 according to the disclosure and Comparative Compound C.

So calculation method: structural optimization calculation by DFT including functional B3LYP, basis function 6-31G (d, p), and toluene solvent effect (PCM);
S₁ calculation method: structural optimization calculation by time-dependent DFT (TDDFT) including functional B3LYP, basis function 6-31G (d, p), and toluene solvent effect (PCM); and
Calculation software used: Gaussian 16 (Gaussian Inc.).

### Simulation Evaluation of Compound

According to “High-Performance Dibenzoheteraborin-Based Thermally Activated Delayed Fluorescence Emitters: Molecular Architectonics for Concurrently Achieving Narrowband Emission and Efficient Triplet-Singlet Spin Conversion”, In Seob Park, Kyohei Matsuo, Naoya Aizawa, and Takuma Yasuda, Advanced Functional Materials 2018, 28, 1802031, the spectrum width of fluorescence (the FWHM of the fluorescence spectrum) has a close relationship with the reorganization energy [E(S₀@S₁)-E(S₀@S₀)] that is expressed by the difference between the ground state (S₀) energy of the stable structure in the first excitation singlet state (S₁) [E(S₀@S₁)] and the ground state (S₀) energy of the stable structure in the ground state (S₀) [E(S₀@S₀)].

Identification of relationship between reorganization energy and spectrum width of fluorescence

First, the relationship between the reorganization energy [E(S₀@S₁)-E(S₀@S₀)] and the spectrum width (FWHM) of fluorescence was identified as follows.

### Calculation by DFT

For the compounds indicated in Tables 1-1 and 1-2, the following calculations were performed by the density functional theory (DFT).

The ground state (S₀) energy of the stable structure in the first excitation singlet state (S₁) [E(S₀@S₁)] and the ground state (S₀) energy of the stable structure in the ground state (S₀) [E(S₀@S₀)] were calculated, and from the difference therebetween, the reorganization energy [E(S₀@S₁)]-[E(S₀@S₀)] (eV) was calculated.

In addition, the first excitation singlet state (S₁) energy of the stable structure in the first excitation singlet state (S₁) [E(S₁@S₁)] was calculated, and from the difference between this value and the ground state (S₀) energy of the stable structure in the ground state (S₀) [E(S₀@S₀)], the adiabatic first excitation singlet state (S₁) energy [E(S₁@S₁)]-[E(S₀@S₀)] (eV) was calculated.

Then, the emission wavelength (nm) obtained by converting the adiabatic first excitation singlet state (S1) energy into a light wavelength (nm) was calculated.

In addition, the highest occupied molecular orbital (HOMO) energy and the lowest unoccupied molecular orbital (LUMO) energy were calculated.

In this regard, calculation by the DFT was performed with Gaussian 16 (Gaussian Inc.) calculation software, according to the following calculation methods (I), (II), and (III):
(I) S₀ calculation method: structural optimization calculation by DFT including functional B3LYP, basis function 6-31G (d, p), and toluene solvent effect (PCM);
(II) S₁ calculation method: structural optimization calculation by time-dependent DFT (TDDFT) including functional B3LYP, basis function 6-31G (d, p), and toluene solvent effect (PCM); and
(III) S₀ calculation method: calculation on input structure by DFT including functional B3LYP, basis function 6-31G (d, p), and toluene solvent effect (PCM).

In detail, the calculation of each item was performed by using the following calculation methods:
Ground state (S₀) energy of stable structure in ground state (S₀) [E(S₀@S₀)]: Calculation method (I);
First excitation singlet state (S₁) energy of stable structure in first excitation singlet state (S₁) [E(S₁@S₁)]: Calculation method (II);
Ground state (S₀) energy of stable structure in first excitation singlet state (S₁) [E(S₀@S₁)]: Calculation methods (II) and (III);
Reorganization energy [E(S₀@S₁)]-[E(S₀@S₀)]: Calculation methods (I), (II), and (III);
Adiabatic first excitation singlet state (S₁) energy [E(S₁@S₁)]-[E(S₀@S₀)]: Calculation methods (I) and (II); and
Emission wavelength (nm): Calculation methods (I) and (II).

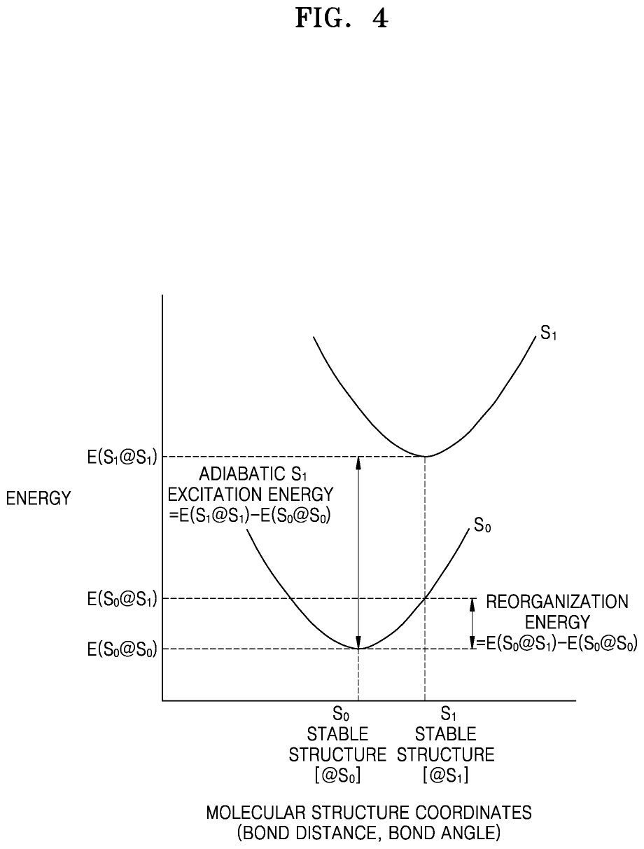

FIG. 4 is an energy level diagram qualitatively illustrating each of the above energy relationships.

### Measurement of Spectrum Width (FWHM) of Fluorescence

For each of toluene solutions respectively including the compounds of Referential Calculation Examples 1 to 3 shown in Table 1-2 at a concentration of 1×10⁻⁵ M (mol/dm³, mol/L), the PL peak wavelength (nm) of fluorescence and the spectrum width of fluorescence (the FWHM of the fluorescence spectrum peak) were measured at room temperature with an excitation wavelength of 320 nm by using spectrofluorophotometer F-7000 manufactured by Hitachi High-Tech Science Co., Ltd.

The calculated values of fluorescence wavelength and reorganization energy and found values of FWHM are shown in Tables 1-1 and 1-2. The compounds of Calculation Examples 1 to 8 shown in Table 1-1 correspond to the compound of Formula (1) according to the disclosure. In addition, the compound of Calculation Example 1 is Compound 1 used in the Examples described below.

TABLE 1-1

| | | Calculated value | | Found |
|---|---|---|---|---|
| | | Emission | Reorganization | value |
| Calculation | Chemical structural | wavelength | energy | FWHM |
| Example | formula | nm | eV | nm |
| Calculation Example 1 | 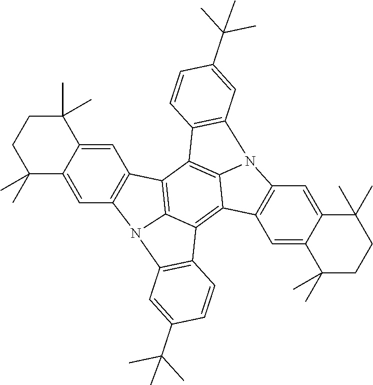 | 443 | 0.064 | — |
| Calculation Example 2 | 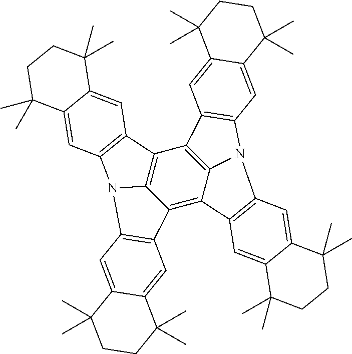 | 449 | 0.055 | — |
| Calculation Example 3 | 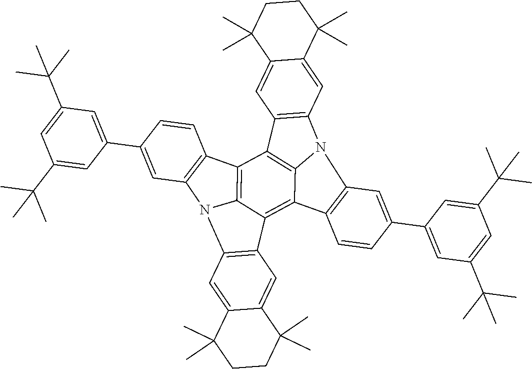 [SMILES: CC(C)(C)c1cc(-c2ccc3c4c5c6cc7c(cc6n6c8cc(-c9cc(C(C)(C)C)cc(C(C)(C)C)c9)ccc8c(c8c9cc%10c(cc9n(c3c2)c48)C(C)(C)CCC%10(C)C)c56)C(C)(C)CCC7(C)C)cc(C(C)(C)C)c1] | 457 | 0.084 | — |
| Calculation Example 4 | 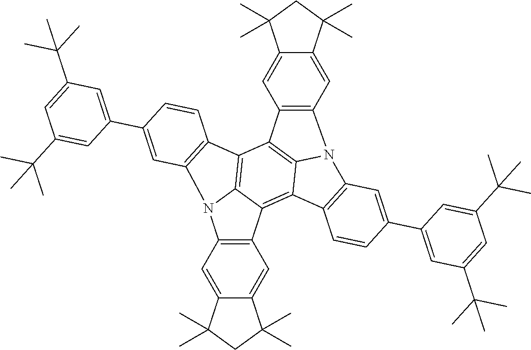 [SMILES: CC(C)(C)c1cc(-c2ccc3c4c5c6cc7c(cc6n6c8cc(-c9cc(C(C)(C)C)cc(C(C)(C)C)c9)ccc8c(c8c9cc%10c(cc9n(c3c2)c48)C(C)(C)CC%10(C)C)c56)C(C)(C)CC7(C)C)cc(C(C)(C)C)c1] | 452 | 0.089 | — |
| Calculation Example 5 | 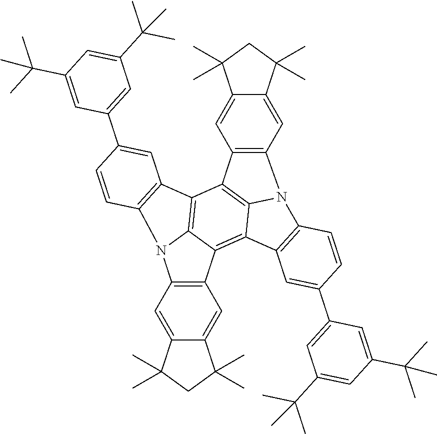 [SMILES: CC(C)(C)c1cc(-c2ccc3c(c2)c2c4c5cc6c(cc5n5c7ccc(-c8cc(C(C)(C)C)cc(C(C)(C)C)c8)cc7c(c7c8cc9c(cc8n3c27)C(C)(C)CC9(C)C)c45)C(C)(C)CC6(C)C)cc(C(C)(C)C)c1] | 451 | 0.057 | — |
| Calculation Example 6 | 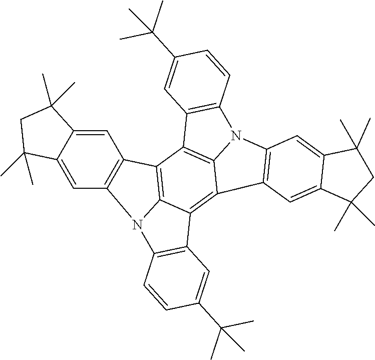 | 442 | 0.048 | — |
| Calculation Example 7 | 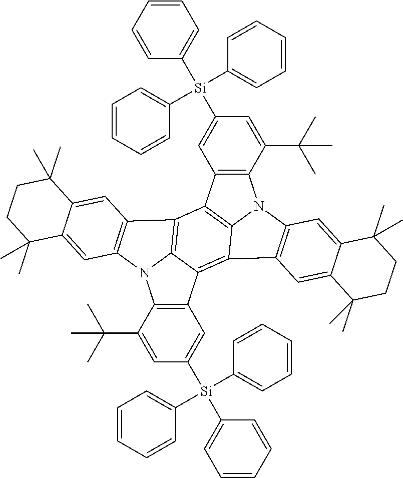 | 435 | 0.063 | — |
| Calculation Example 8 | 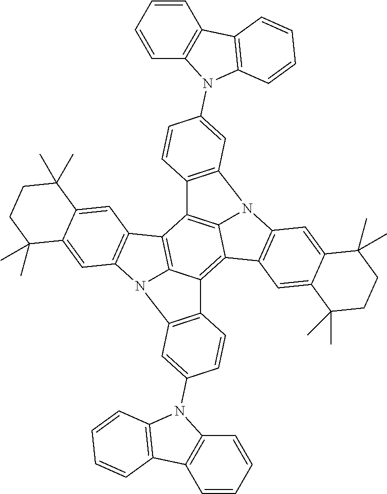 | 452 | 0.096 | — |

TABLE 1-2

| | | Calculated value | | Found |
|---|---|---|---|---|
| | | Emission | Reorganization | value |
| Calculation | Chemical structural | wavelength | energy | FWHM |
| Example | formula | nm | eV | nm |
| Referential Calculation Example 1 | 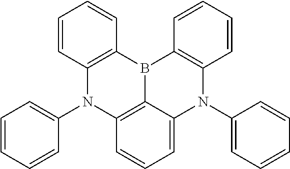 [SMILES: c1ccc(N2c3ccccc3B3c4ccccc4N(c4ccccc4)c4cccc2c43)cc1] | 423 | 0.109 | 22 |
| Referential Calculation Example 2 | 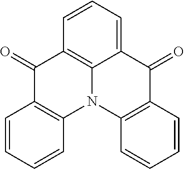 [SMILES: O=c1c2ccccc2n2c3ccccc3c(=O)c3cccc1c32] | 436 | 0.132 | 26 |
| Referential Calculation Example 3 | 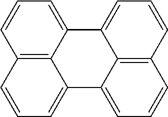 [SMILES: c1cc2cccc3c4cccc5cccc(c(c1)c23)c54] | 495 | 0.164 | 42 |
| Comparative Calculation Example 1 | 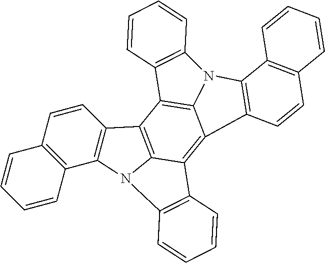 [SMILES: c1ccc2c(c1)ccc1c3c4c5ccccc5n5c6c7ccccc7ccc6c(c6c7ccccc7n(c21)c63)c45] | 471 | 0.131 | — |
| Comparative Calculation Example 2 | 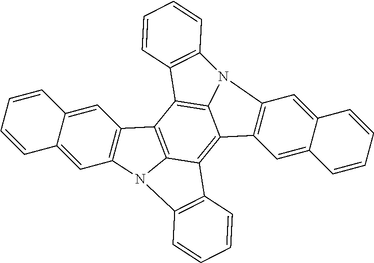 [SMILES: c1ccc2cc3c(cc2c1)c1c2c4ccccc4n4c5cc6ccccc6cc5c(c5c6ccccc6n3c51)c24] | 502 | 0.064 | — |
| Comparative Calculation Example 3 | 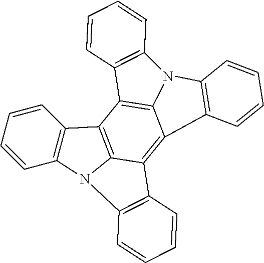 [SMILES: c1ccc2c(c1)c1c3c4ccccc4n4c5ccccc5c(c5c6ccccc6n2c15)c34] | 437 | 0.055 | — |

### Emission Spectrum of Solution

### Measurement Method

For each of toluene solutions respectively including the compounds at a concentration of 1×10⁻⁵ M (mol/dm³, mol/L), the PL peak wavelength (nm) of fluorescence and the spectrum width of fluorescence (the FWHM of the fluorescence spectrum peak) were evaluated by measuring at room temperature with an excitation wavelength of 320 nm by using spectrofluorophotometer F-7000 manufactured by Hitachi High-Tech Science Co., Ltd.

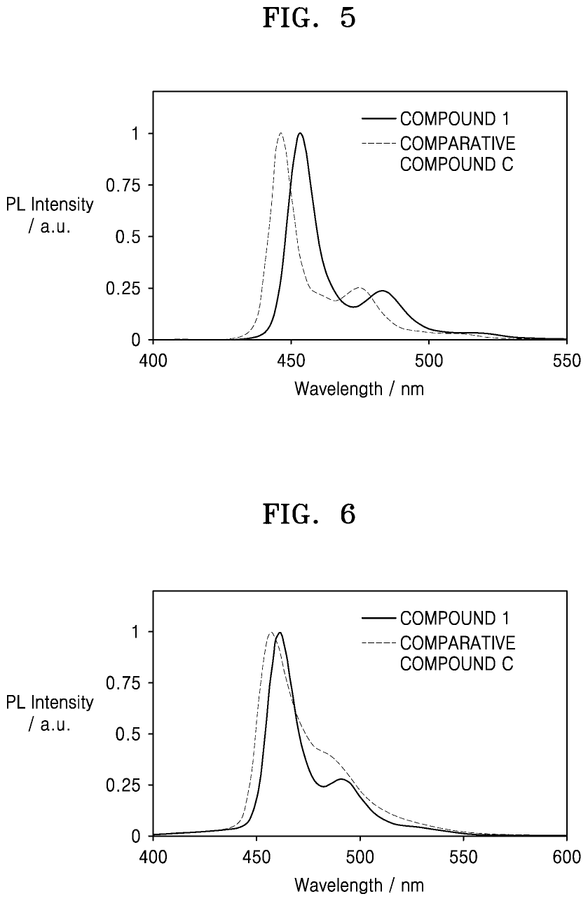

The emission spectra of a toluene solution according to the measurement method with respect to Compound 1 and Comparative Compound C are shown in FIG. 5 and summarized in Table 2. The emission wavelength of Compound 1 was 454 nm to provide emission of blue light. In addition, the FWQM of Compound 1 was 19 nm which was 16 nm less than that of Comparative Compound C. This is because the second emission peak intensity was suppressed by the effects of condensed rings introduced to Compound 1.

TABLE 2
Summary of emission spectrum of toluene solution

| Compound | λ^{PL}/nm | FWHM/nm | FWQM/nm |
|---|---|---|---|
| Compound 1 | 454 | 12 | 19 |
| Comparative Compound C | 446 | 12 | 35 |

### Properties of Thin Film

### Manufacturing Method of Thin Film

The compound indicated in Table 3 was co-deposited on a quartz substrate at a weight ratio of 1 wt % based on the total weight of the host compound at a vacuum degree of 10⁻⁵ pascals (Pa) to prepare a thin film with a thickness of 50 nm. In this regard, Compounds HT1 and HT2 were used as the host compound, and the weight ratio between Compound HT1 and Compound HT2 was 60:40. In addition, the structures of Compounds HT1 and HT2 are as follows:

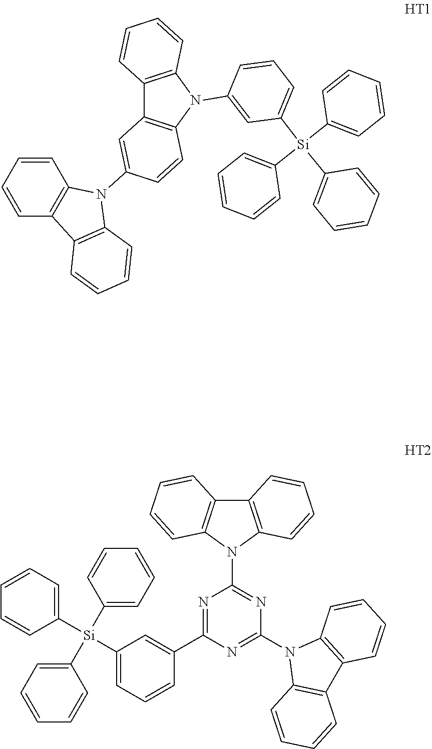 [SMILES: c1ccc([Si](c2ccccc2)(c2ccccc2)c2cccc(-n3c4ccccc4c4cc(-n5c6ccccc6c6ccccc65)ccc43)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1]

### PL Measurement (FWHM, FWQM)

The manufactured thin film was cut into a flat shape with a width of 6 millimeters (mm), and the PL measurement was performed at room temperature with a spectrofluorophotometer F-7000 of Hitachi Hightech Inc. From the obtained emission spectrum, a peak wavelength, a wavelength width at which an emission intensity is halved (FWHM), and a wavelength width corresponding to a quarter of a maximum value (FWQM) were defined. The evaluation results thereof are shown in Table 3.

### Measurement of PL Quantum Yield (PLQY)

PLQY of the manufactured thin film was measured with a Quantaurus-QY absolute PLQY measurement device C11347-01 manufactured by Hamamatsu Photonics Co., Ltd. In the measurement, the excitation wavelength was measured by scanning at intervals of 10 nm from 280 nm to 350 nm, and the excitation wavelength region in which the compound absorption value showed 20% or more of the excitation light intensity ratio was adopted. The value of PLQY was taken as the highest value in the adopted excitation wavelength region. The evaluation results thereof are shown in Table 3.

The optical characteristics of a host dispersion film were evaluated. The emission spectrum of an evaporated thin film using HT1 and HT2 as host molecule is shown in FIG. 6 and summarized in Table 3. The emission wavelength of Compound 1 was 460 nm to provide emission of blue light. Moreover, the FWHM of emission spectrum was 17 nm, which did not show much difference when a solution was applied, in a dispersed state of host, a newly introduced cycloalkane ring with a sterically large volume may effectively suppress aggregation of molecules, which leads to a reduced FWHM. Although the emission wavelength of Comparative Compound C was 457 nm, which provides blue light emission, the FWHM was 24 nm, which is considered a wide value, and the spectrum shape thereof was different than that of the solution of Compound 1. As Comparative Compound C is a planar molecule, aggregation among Compound C may occur, resulting in increased FWQM. In addition, as the PLQY of Compound 1 was 11% greater than that of Comparative Compound C, the effect of suppressing aggregation may be more easily achieved, and Compound 1, which is the compound of Formula (1) may be an improved dopant.

TABLE 3
Summary of emission spectrum of host
dispersion evaporated thin film

| Compound | λ^{PL}/nm | FWHM/nm | FWQM/nm | PLQY/% |
|---|---|---|---|---|
| Compound 1 | 460 | 17 | 32 | 76 |
| Comparative | 457 | 24 | 51 | 65 |
| Compound C | | | | |

### Evaluation of Organic Light-Emitting Devices (OLED)

### Manufacturing of Organic EL Device

### Preparation of Material for Forming Each Layer

For the material for forming each layer of the organic EL device, the materials below were prepared in addition to the obtained Compound 1 and Comparative Compound C. The phosphorescent complex Pt1 is the same as the phosphorescent complex P120 described above.

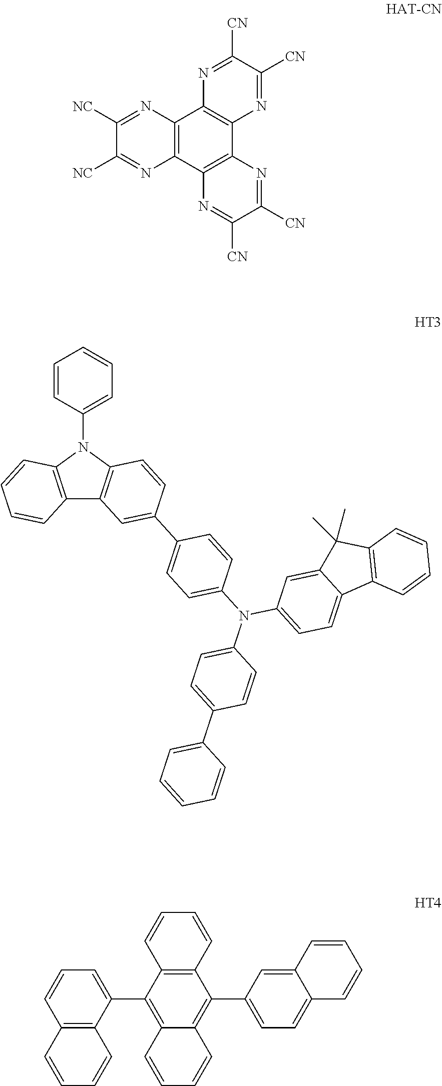

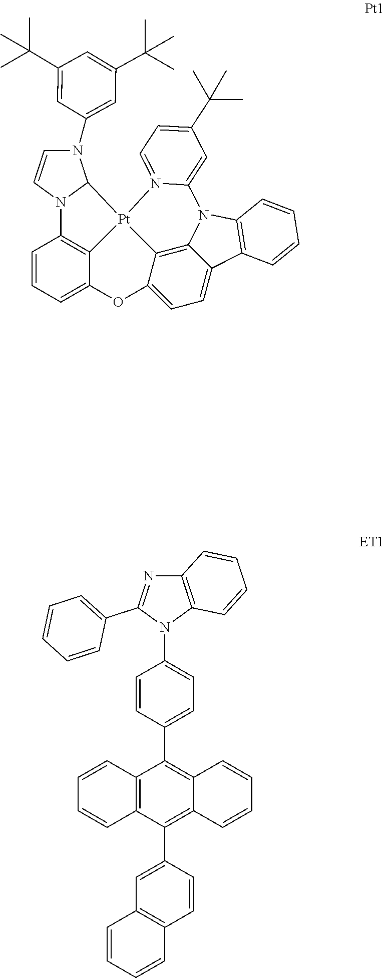

### Manufacture 1 of Organic EL Device

### Example 1

An electrode-patterned ITO glass substrate was cut to a size of 50 mm×50 mm×0.7 mm, sonicated with acetone, isopropyl alcohol, and pure water, in this stated order, each for 15 minutes, and then cleaned by exposure to UV ozone for 30 minutes. The following layers were deposited on the ITO electrode (anode) of the glass substrate.

First, HAT-CN was deposited on the ITO electrode to form a hole injection layer with a thickness of 10 nm. Next, Compound HT3 was deposited on the hole injection layer to form a hole transport layer with a thickness of 140 nm. Then, Compound HT1 was deposited on the hole transport layer to form an electron blocking layer with a thickness of 5 nm. As a result, a hole transport region was formed.

Compound HT1, Compound HT2, and Compound 1 obtained above were co-deposited on the hole transport region obtained above to form an emission layer with a thickness of 40 nm. The formation of the emission layer was performed such that the weight ratio of Compound HT1 and Compound HT2 in the emission layer was Compound HT1: Compound HT2=60:40. In addition, the formation of the emission layer was performed such that the concentration of Compound 1 was 1.5 wt % based on the total weight of Compound HT1, Compound HT2, and Compound 1 (that is, the total weight of the emission layer). Compounds HT1 and HT2 are host materials.

Compound HT2 was vacuum-deposited on the emission layer obtained above to form a hole blocking layer with a thickness of 5 nm. Next, Compound ET1 and LiQ were co-deposited on the hole blocking layer at a weight ratio of Compound ET1:LiQ=5:5 (unit: parts by weight) to form an electron transport layer with a thickness of 30 nm. Then, LiQ was deposited on the electron transport layer to form an electron injection layer with a thickness of 1 nm. As a result, an electron transport region was formed.

Al (cathode) with a thickness of 100 nm was deposited on the electron injection layer to thereby manufacture an organic EL device.

Then, in a glove box under a nitrogen atmosphere with a water concentration of 1 parts per million (ppm) or less and an oxygen concentration of 1 ppm or less, a glass sealing tube with a desiccating agent and an ultraviolet curing resin (manufactured by MORESCO, product name WB90US) were used to seal the organic EL device manufactured by the above process. As a result, the manufacturing of the organic EL device was completed.

### Comparative Example 1

The organic EL device was manufactured in the same manner as in Example 1, except that, when forming the emission layer, Comparative Compound C was used in place of Compound 1 in the emission layer.

### Manufacture 2 of Organic EL Device

### Example 2

The organic EL device was manufactured in the same manner as in Example 1, except that, when forming the emission layer, Compound HT4 was used in place of Compounds HT1 and HT2 in the emission layer.

### Manufacture 3 of Organic EL Device

### Example 3

An electrode-patterned ITO glass substrate was cut to a size of 50 mm×50 mm×0.7 mm, sonicated with acetone, isopropyl alcohol, and pure water, in this stated order, each for 15 minutes, and then cleaned by exposure to UV ozone for 30 minutes. The following layers were deposited on the ITO electrode (anode) of the glass substrate.

First, HAT-CN was deposited on the ITO electrode to form a hole injection layer with a thickness of 10 nm. Next, Compound HT3 was deposited on the hole injection layer to form a hole transport layer with a thickness of 140 nm. Then, Compound HT1 was deposited on the hole transport layer to form an electron blocking layer with a thickness of 5 nm. As a result, a hole transport region was formed.

Compound HT1, Compound HT2, Phosphorescent Complex Pt1, and Compound 1 obtained above were co-deposited on the hole transport region obtained above to form an emission layer with a thickness of 40 nm. The formation of the emission layer was performed such that the weight ratio of Compound HT1, Compound HT2, and Phosphorescent Complex Pt1 in the emission layer was Compound HT1: Compound HT2: Phosphorescent Complex Pt1=60:40:13. In addition, the formation of the emission layer was performed such that the concentration of Compound 1 was 0.4 wt % based on the total weight of Compound HT1, Compound HT2, Phosphorescent Complex Pt1, and Compound 1 (that is, the total weight of the emission layer). Compounds HT1 and HT2 are host materials.

The subsequent lamination process and encapsulation process were performed as in Example 1.

### Comparative Example 2

The organic EL device was manufactured in the same manner as in Example 3, except that, when forming the emission layer, Comparative Compound C was used in place of Compound 1 in the emission layer.

### Evaluation of Organic EL Device

### Luminance, External Quantum Yield, and Device Lifespan

The peak wavelength of luminescence, spectrum width of luminescence, external quantum yield, and device lifespan at a luminance of 1,000 candela per square meter (cd/m²) were evaluated according to the following method, and the results thereof are shown in Table 4.

A direct current regulated power supply (Source Meter 2400 made by KEITHLEY) was used to change the applied voltage with respect to the organic EL device while emitting light, and the luminance, emission spectrum and the luminescence amount were measured in the luminance measurement apparatus (SR-3 made by Topcon).

Here, the external quantum efficiency was calculated from the luminescence amount of the emission spectrum, luminance, and the current value at the time of measurement. The external quantum efficiency at a luminance of 1,000 cd/m² is defined as EQE[%], and the external quantum efficiency at a current density of 0.1 milliampere per square centimeter (mA/cm²) is defined as MaxEQE[%].

The device lifespan (durability) was defined as LT95[hr] by measuring the amount of time taken when the emission luminance, which decays as time lapses, becomes 95% of the initial luminance when the device is continuously driven on a current value with an initial luminance of 1,000 cd/m². The device lifespan (LT95) in Table 4 was shown as a relative value based on the device lifespan (LT95) of Comparative Example 1, and similarly, the device lifespan (LT95) in Table 5 was shown as a relative value based on the device lifespan (LT95) of Comparative Example 2.

### Emission Peak Wavelength and Emission Spectrum Width (FWHM, FWQM)

The peak wavelength of luminescence and spectrum width of luminescence were determined from the emission spectrum. A wavelength showing a maximum value of emission spectrum is defined as an emission peak wavelength, a wavelength width corresponding to a half of peak height is defined as a FWHM, and a wavelength width corresponding to a quarter of peak height is defined as a FWQM.

In this evaluation, the emission peak wavelength is not particularly limited, but may be within the blue emission region, and may be 455 nm or more and 475 nm or less.

In this evaluation, a narrower emission spectrum width (FWHM and FWQM) is preferred and regarded as providing higher color purity.

The evaluation results of OLED are shown in Tables 4 and 5. An OLED manufactured with Compound 1 of the disclosure as a luminescent material provided higher emission quantum yield and longer device lifespan than an OLED manufactured with Comparative Compound C as a luminescent material. These are effects that may be achieved by including a substituent with a sterically large volume according to the disclosure, which may cause an increase in efficiency due to suppression of aggregation. Also, it is clearly understood that the substituent of the disclosure contributes to a longer lifespan of device. Moreover, both of the FWHM and FWQM were narrower for Example 1 than in Comparative Example 1, and it was confirmed that the compound of the disclosure was an excellent dopant.

TABLE 4
Results for OLED

| | | External | Device | FWHM/ | FWQM/ |
|---|---|---|---|---|---|
| | Compound | quantum yield | lifespan | nm | nm |
| Example 1 | Compound 1 | 1.5 | 3.6 | 19 | 53 |
| Example 2 | Compound 1 | 2.3 | 1.0 | 19 | 44 |
| Comparative | Comparative | 1.0 | 1.0 | 25 | 60 |
| Example 1 | Compound C | | | | |

### External Quantum Yield and Device Lifespan are Shown in Relative Values Based on a Reference Value of Comparative Example 1.

TABLE 5
Results for OLED

| | | External | Device | FWHM/ | FWQM/ |
|---|---|---|---|---|---|
| | Compound | quantum yield | lifespan | nm | nm |
| Example 3 | Compound1 | 1.7 | 1.8 | 18 | 44 |
| Comparative | Comparative | 1.0 | 1.0 | 20 | 56 |
| Example 2 | Compound C | | | | |

### External Quantum Yield and Device Lifespan are Shown in Relative Values Based on a Reference Value of Comparative Example 2.

As shown in Tables 4 and 5, in the organic EL devices of Examples 1 and 2 which include the compound according to the disclosure, the FWHM of emission spectrum became narrower, and high-color purity emission was realized. In addition, the external quantum yield was increased (50%, 130%, and 70% greater for Examples 1, 2, and 3, respectively). The device lifespan for Examples 1 to 3 was the same or greater than the Comparative Examples. That is, by using the compound of the disclosure as a luminescent material, a blue EL device of narrower spectrum width, higher performance, higher efficiency, and higher color purity in comparison to one manufactured with conventional luminescent materials may be obtained.

As such the compound according to the disclosure demonstrated precisely adjusted blue light color, excellent color purity, and high luminescence efficiency in an organic EL device. Moreover, when the compound is used along with the phosphorescent complex, the device lifespan showed significant improvement. Such results sufficiently satisfy the specifications required for future optical color gamut devices based on BT2100, etc., and may provide next-generation high-precision display devices.

According to an embodiment, providing a compound wherein the peak wavelength of the emission spectrum is within the blue wavelength region, can provide high color purity, high luminescence efficiency, and long lifespan. In addition, according to another embodiment, an organic EL device including such compound may be provided. Moreover, providing a method wherein the peak wavelength of the emission spectrum in the organic EL device is within a blue wavelength region, can provide high color purity, high luminescence efficiency, and long lifespan.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A compound represented by Formula (1): 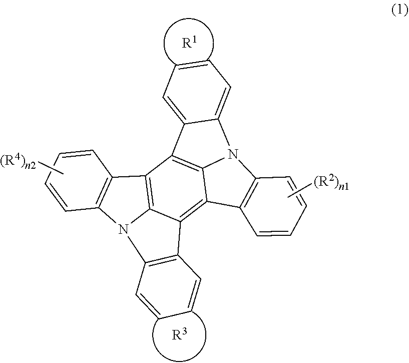
wherein, in Formula (1),
R¹ and R³ are each independently a saturated hydrocarbon group or a saturated heterocyclic group, each including at least one substituent and having 5 to 9 ring-forming atoms,
R² and R⁴ are each independently any one atom or group selected from (a1) to (a8):
(a1) a halogen atom;
(a2) a cyano group;
(a3) a substituted or unsubstituted alkyl group having 1 to 20 carbon atoms;
(a4) a substituted or unsubstituted alkoxy group having 1 to 20 carbon atoms;
(a5) a substituted or unsubstituted arylamino group having 6 to 20 carbon atoms;
(a6) a substituted or unsubstituted triarylsilyl group, a substituted or unsubstituted alkyldiarylsilyl group, a substituted or unsubstituted dialkylarylsilyl group, or a substituted or unsubstituted trialkylsilyl group, wherein an aryl group has 6 to 20 carbon atoms, and an alkyl group has 1 to 20 carbon atoms;
(a7) a substituted or unsubstituted aromatic hydrocarbon group having 6 to 30 carbon atoms; and
(a8) a substituted or unsubstituted heterocyclic group having 3 to 30 ring-forming atoms,
wherein n1 and n2 are each independently 0, 1, 2, 3, or 4, and when n1 is 2 or more, each R² is identical to or different from each other, and when n2 is 2 or more, each R⁴ is identical to or different from each other, and
(a9) when n1 is 2 or more, two R² optionally bond with each other to form a substituted or unsubstituted saturated hydrocarbon group or saturated heterocyclic group having 5 to 9 ring-forming atoms, and when n2 is 2 or more, two R⁴ optionally bond with each other to form a substituted or unsubstituted saturated hydrocarbon group or saturated heterocyclic group having 5 to 9 ring-forming atoms.

2. The compound of claim 1, wherein at least one substituent of R¹ and R³ is selected from a halogen atom, a cyano group, a substituted or unsubstituted alkyl group having 1 to 20 carbon atoms, an unsubstituted haloalkyl group having 1 to 20 carbon atoms, an unsubstituted alkoxy group having 1 to 20 carbon atoms, an unsubstituted alkylamino group having 1 to 20 carbon atoms, an unsubstituted arylamino group having 6 to 20 carbon atoms, a substituted or unsubstituted aromatic hydrocarbon group having 6 to 30 carbon atoms, and an unsubstituted heterocyclic group having 3 to 30 ring-forming atoms.

3. The compound of claim 1, wherein substituents substituting the groups of (a3) to (a9) are each independently at least one substituent selected from a halogen atom, a cyano group, a substituted or unsubstituted alkyl group having 1 to 20 carbon atoms, an unsubstituted haloalkyl group having 1 to 20 carbon atoms, an unsubstituted alkoxy group having 1 to 20 carbon atoms, an unsubstituted alkylamino group having 1 to 20 carbon atoms, an unsubstituted arylamino group having 6 to 20 carbon atoms, a substituted or unsubstituted aromatic hydrocarbon group having 6 to 30 carbon atoms, and an unsubstituted heterocyclic group having 3 to 30 ring-forming atoms.

4. The compound of claim 1, wherein n1 and n2 are each independently 0, 1, or 2.

5. The compound of claim 1, wherein R¹ and R³ in the compound represented by Formula (1) have a structure represented by Formula (2): 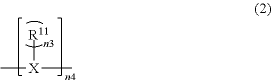
wherein, in Formula (2),
R¹¹ are independently any one atom or group selected from (b1) to (b7):
(b1) a hydrogen atom or a deuterium atom;
(b2) a cyano group;
(b3) a substituted or unsubstituted alkyl group having 1 to 20 carbon atoms;
(b4) a substituted or unsubstituted alkoxy group having 1 to 20 carbon atoms;
(b5) a substituted or unsubstituted arylamino group having 6 to 20 carbon atoms;
(b6) a substituted or unsubstituted aromatic hydrocarbon group having 6 to 30 carbon atoms; and
(b7) a substituted or unsubstituted heterocyclic group having 6 to 30 ring-forming atoms,
wherein at least one of R¹¹ is selected from (b2) to (b7),
wherein at least one of X is a carbon atom, X are each independently a carbon atom, a silicon atom, a nitrogen atom, an oxygen atom, a sulfur atom, or a selenium atom, and a plurality of X are identical to or different from each other, and
wherein n3 are each independently 0, 1, or 2, and when n3 is 2, R¹¹ are identical to or different from each other, and
n4 is 3, 4, 5, 6, or 7.

6. The compound of claim 1, wherein the compound represented by Formula (1) has a structure represented by Formula (1A) or (1B): 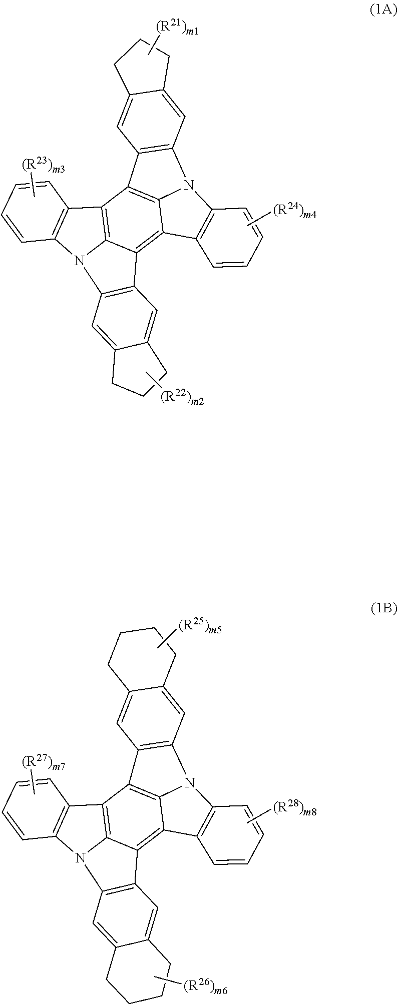
wherein, in Formulae (1A) and (1B),
R²¹, R²², R²⁵, and R²⁶ are each independently any one atom or group selected from (c1) to (c6):
(c1) a cyano group;
(c2) a substituted or unsubstituted alkyl group having 1 to 20 carbon atoms;
(c3) a substituted or unsubstituted alkoxy group having 1 to 20 carbon atoms;
(c4) a substituted or unsubstituted arylamino group having 6 to 20 carbon atoms;
(c5) a substituted or unsubstituted aromatic hydrocarbon group having 6 to 30 carbon atoms; and
(c6) a substituted or unsubstituted heterocyclic group having 6 to 30 ring-forming atoms,
wherein R²³, R²⁴, R²⁷, and R²⁸ are each independently any one atom or group selected from (d1) to (d9):
(d1) a halogen atom;
(d2) a cyano group;
(d3) a substituted or unsubstituted alkyl group having 1 to 20 carbon atoms;
(d4) a substituted or unsubstituted alkoxy group having 1 to 20 carbon atoms;
(d5) a substituted or unsubstituted arylamino group having 6 to 20 carbon atoms;
(d6) a substituted or unsubstituted triarylsilyl group, a substituted or unsubstituted alkyldiarylsilyl group, a substituted or unsubstituted dialkylarylsilyl group, or a substituted or unsubstituted trialkylsilyl group, wherein an aryl group has 6 to 20 carbon atoms, and an alkyl group has 1 to 20 carbon atoms;
(d7) a substituted or unsubstituted aromatic hydrocarbon group having 6 to 30 carbon atoms;
(d8) a substituted or unsubstituted heterocyclic group having 3 to 30 ring-forming atoms; and
(d9) a substituted or unsubstituted saturated hydrocarbon group or saturated heterocyclic group having 5 to 9 ring-forming atoms and formed by two R²³ bonded to each other when m3 is 2, a substituted or unsubstituted saturated hydrocarbon group or saturated heterocyclic group having 5 to 9 ring-forming atoms and formed by two R²⁴ bonded to each other when m4 is 2, a substituted or unsubstituted saturated hydrocarbon group or saturated heterocyclic group having 5 to 9 ring-forming atoms and formed by two R²⁷ bonded to each other when m7 is 2, and a substituted or unsubstituted saturated hydrocarbon group or saturated heterocyclic group having 5 to 9 ring-forming atoms and formed by two R²⁸ bonded to each other when m8 is 2, and
wherein m1 and m2 are each independently 0, 1, 2, 3, 4, 5 or 6, and when m1 is 2 or more, R²¹ are identical to or different from each other, and when m2 is 2 or more, R²² are identical to or different from each other,
m5 and m6 are each independently 0, 1, 2, 3, or 4, and when m5 is 2 or more, R²⁵ are identical to or different from each other, and when m6 is 2 or more, R²⁶ are identical to or different from each other, and
m3, m4, m7, and m8 are each independently 0, 1, 2, 3, or 4, and when m3 is 2 or more, R²³ are identical to or different from each other, when m4 is 2 or more, R²⁴ are identical to or different from each other, when m7 is 2 or more, R²⁷ are identical to or different from each other, and when m8 is 2 or more, R²⁸ are identical to or different from each other.

7. The compound of claim 1, wherein the compound represented by Formula (1) has a structure represented by any one of Formulae (1A-1), (1A-2), and (1B-1): 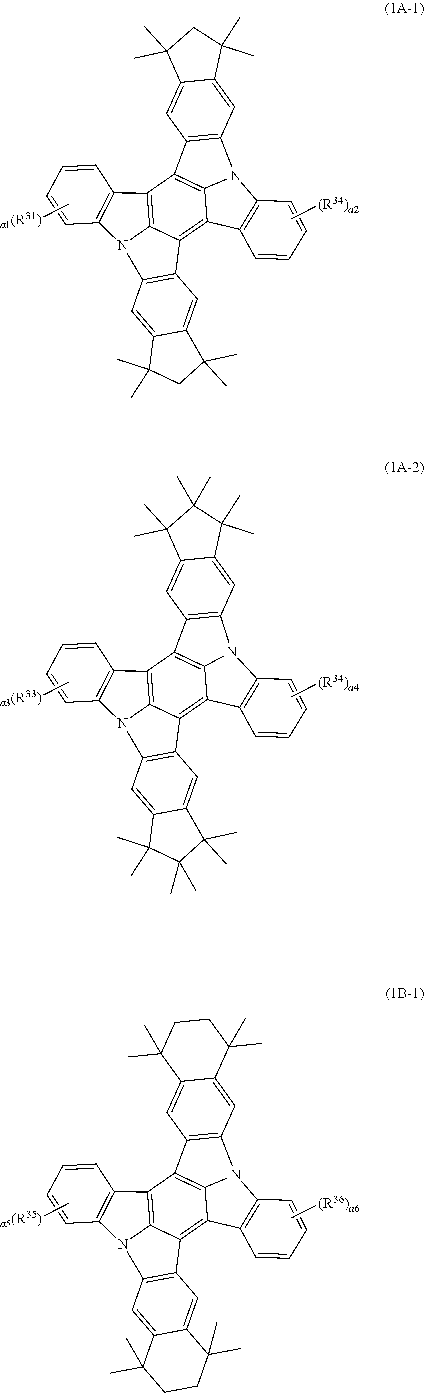
wherein, in Formulae (1A-1), (1A-2), and (1B-1),
R³¹ to R³⁶ are each independently any one atom or group selected from (d1) to (d9), and
a1 to a6 are each independently 0, 1, 2, 3, or 4, and when a1 is 2 or more, R³¹ are identical to or different from each other, when a2 is 2 or more, R³² are identical to or different from each other, when a3 is 2 or more, R³³ are identical to or different from each other, when a4 is 2 or more, R³⁴ are identical to or different from each other, when a5 is 2 or more, R³⁵ are identical to or different from each other, and when a6 is 2 or more, R³⁶ are identical to or different from each other.

8. The compound of claim 6, wherein R²³, R²⁴, R²⁷, and R²⁸ are any one group selected from Group (X): 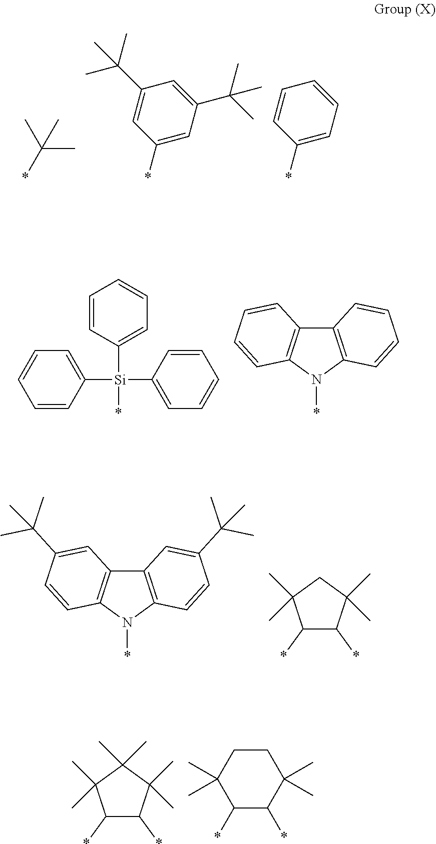
“*” in a structural formula of Group (X) indicates a binding site to a benzene ring, and
a case where two “*” are present in a structural formula of Group (X) indicates that R²³, R²⁴, R²⁷, and R²⁸ condense with the benzene ring where they are substituted.

9. The compound of claim 1, wherein the compound represented by Formula (1) is any one of Compounds (100) to (115) and (117) to (131):

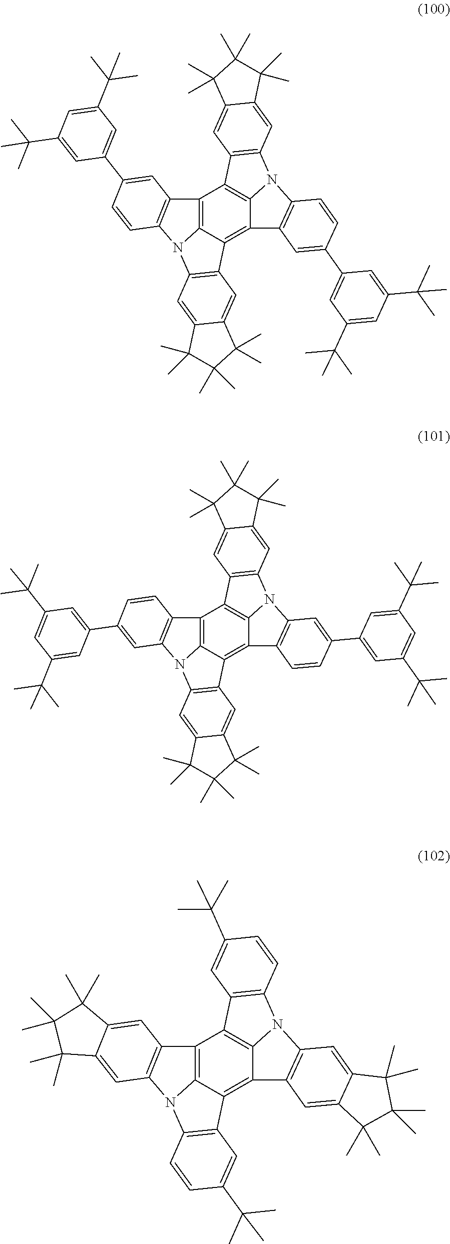

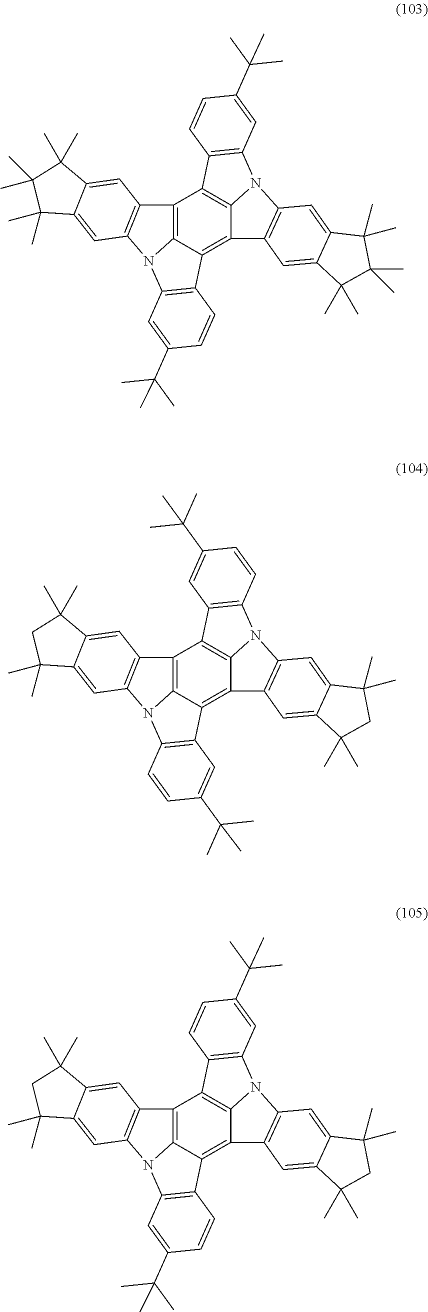

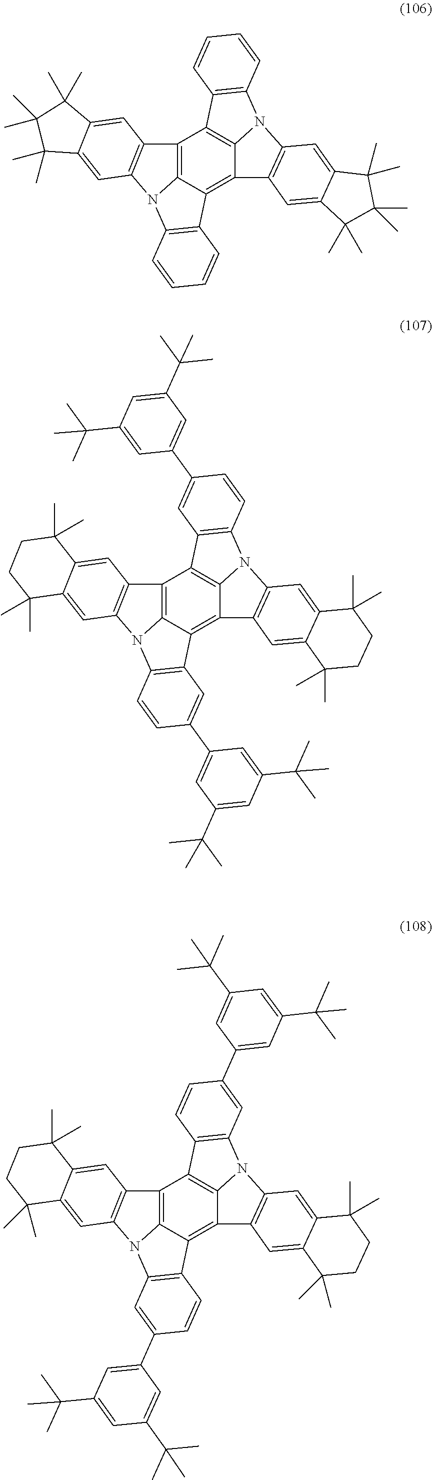

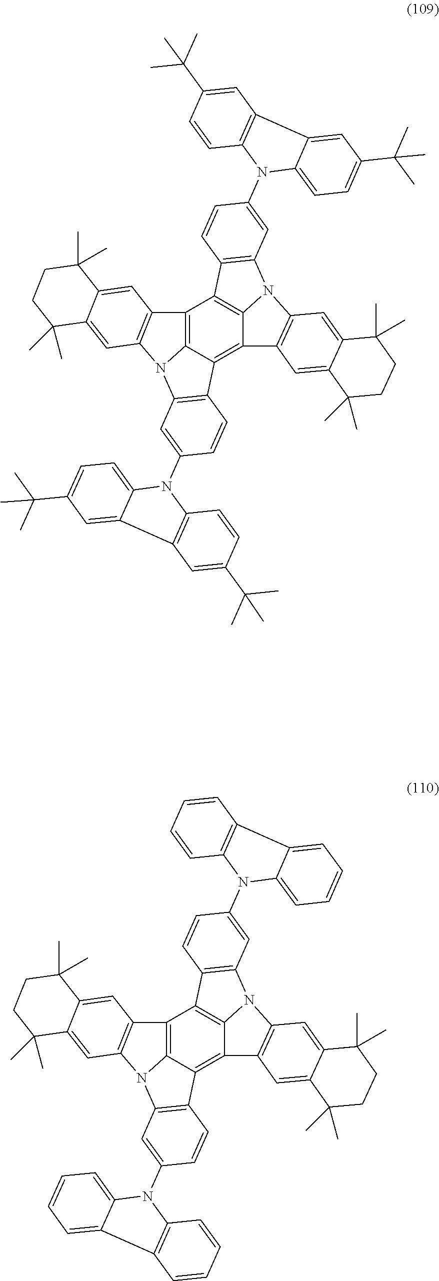

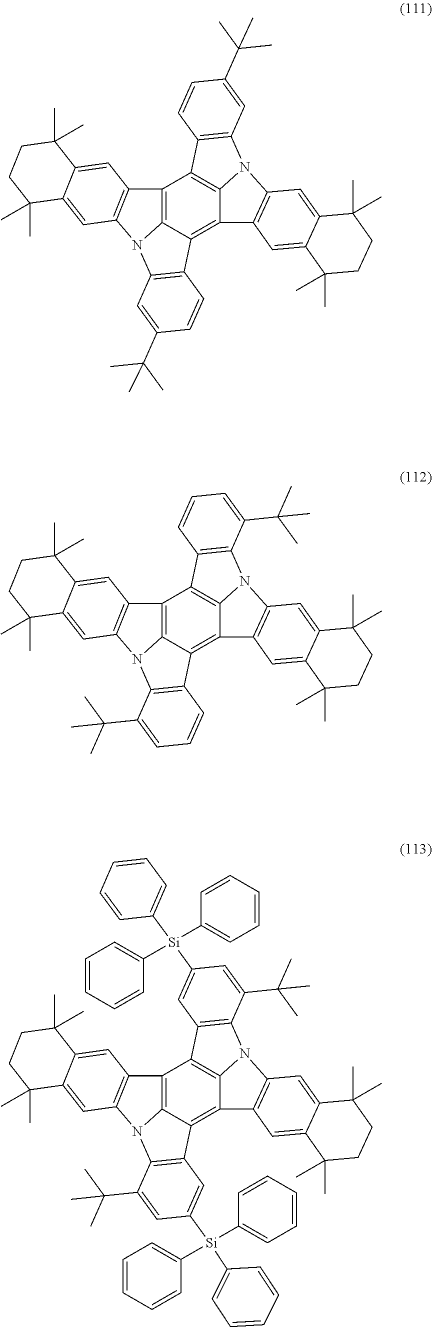

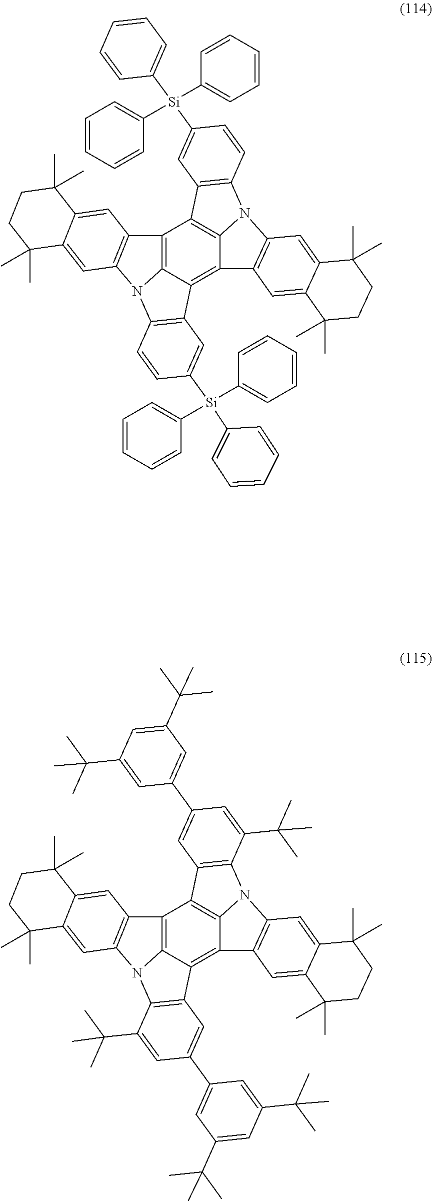

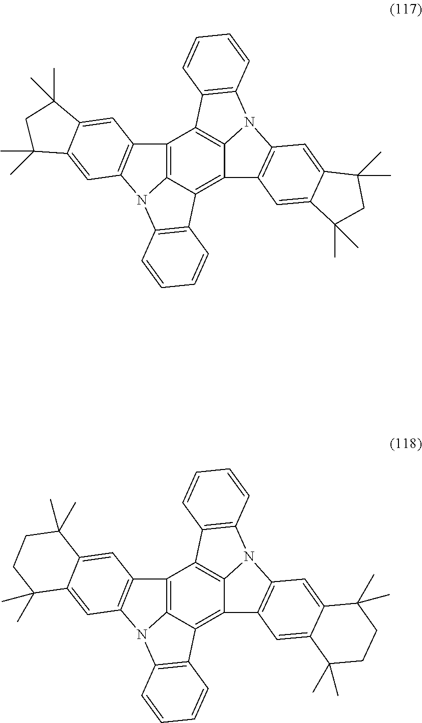

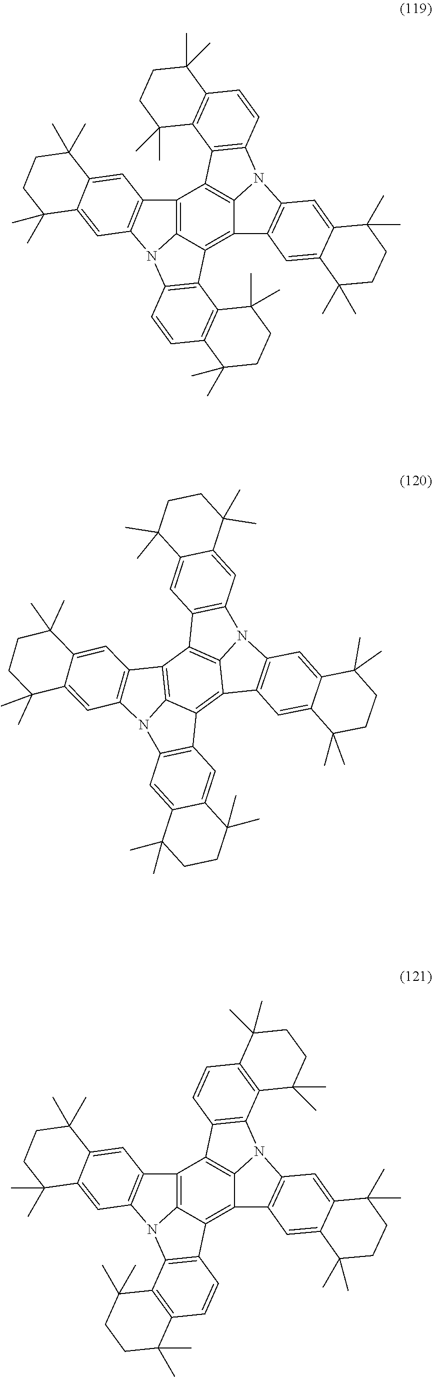

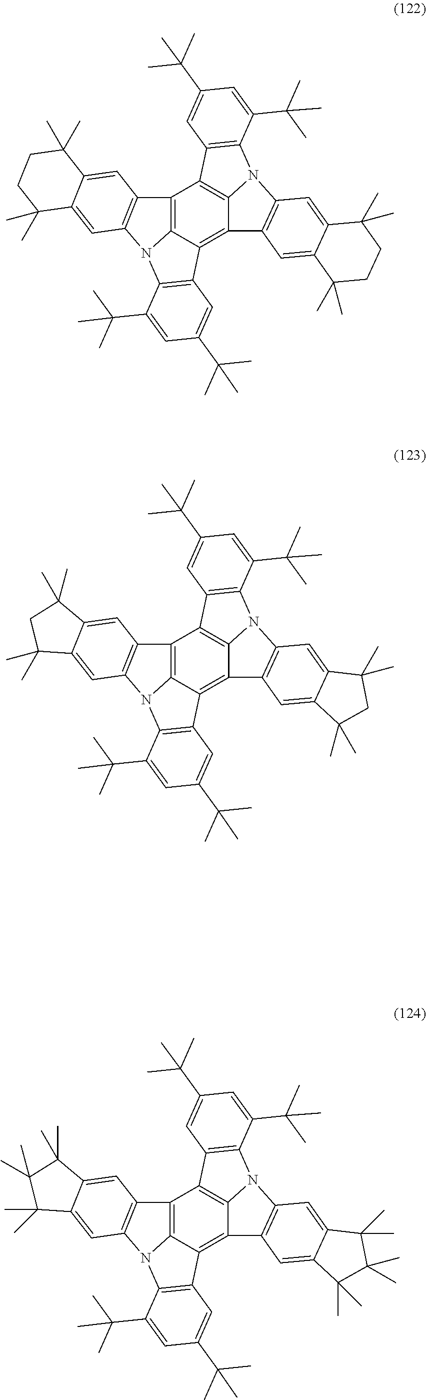

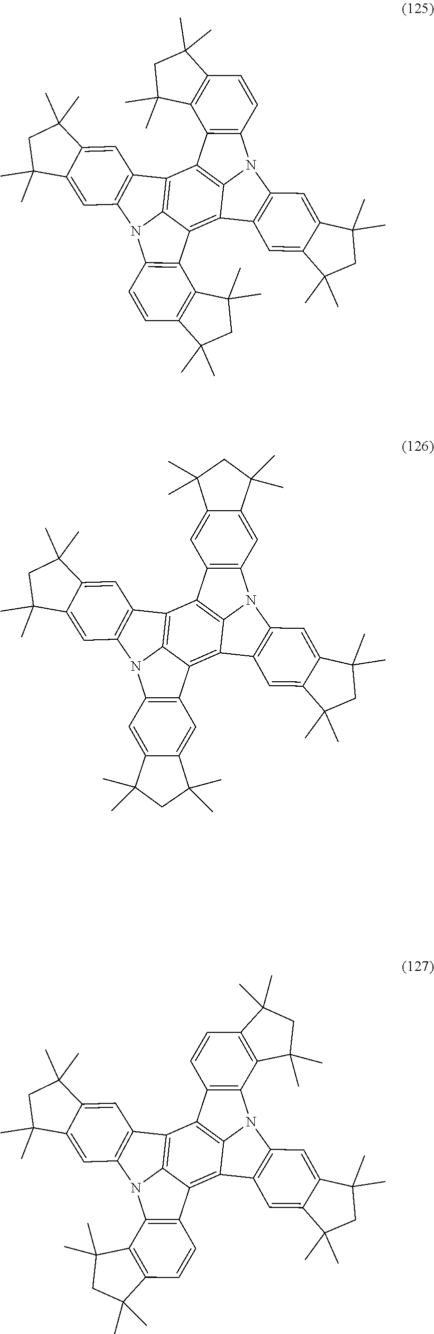

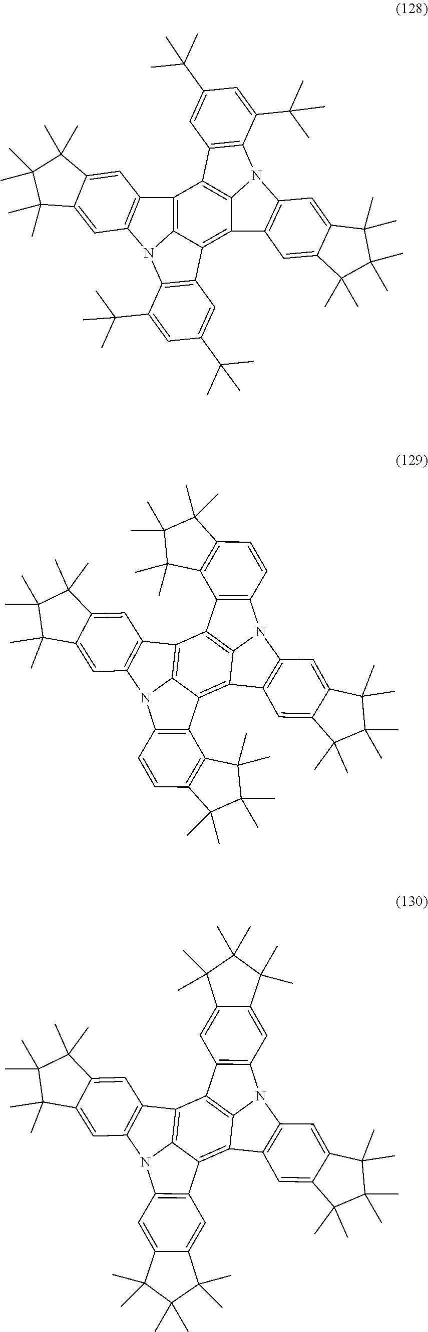

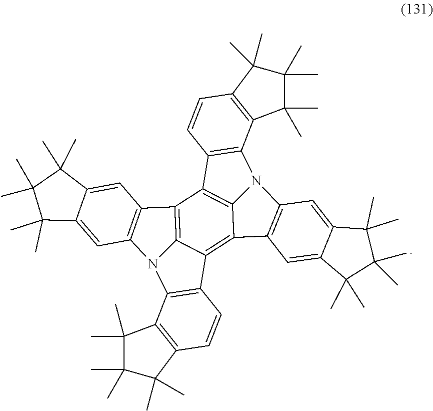

10. An organic electroluminescent device comprising an emission layer comprising the compound according to claim 1.

11. The organic electroluminescent device of claim 10, wherein the emission layer further comprises a phosphorescent complex.

12. The organic electroluminescent device of claim 11, wherein the phosphorescent complex is a platinum complex.

13. The organic electroluminescent device of claim 11, wherein the phosphorescent complex is a compound having a structure represented by Formula (4): 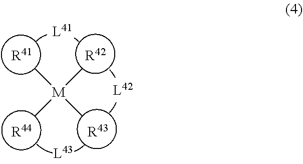
wherein, in Formula (4), M is a metal ion having a coordination number of 4,
R⁴¹, R⁴², R⁴³, and R⁴⁴ are each independently a substituted or unsubstituted hydrocarbon cyclic group having 6 to 30 carbon atoms or a substituted or unsubstituted heterocyclic group having 3 to 30 ring-forming atoms,
L⁴¹ is a linking group linking R⁴¹ and R⁴²,
L⁴² is a linking group linking R⁴² and R⁴³, and
L⁴³ is a linking group linking R⁴³ and R⁴⁴.

14. The organic electroluminescent device of claim 10, wherein the emission layer further comprises a host material.

15. The organic electroluminescent device of claim 14, wherein the host material comprises a compound having a carbazole ring structure or a compound having a ring structure in which at least one ring-forming carbon atom of a carbazole ring is substituted with a nitrogen atom.

16. The organic electroluminescent device of claim 14, wherein the host material comprises a compound having a structure represented by Formula (5): 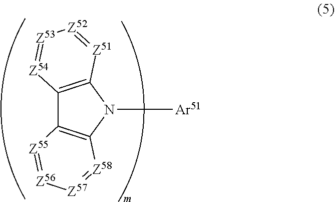
wherein, in Formula (5),
Z⁵¹ is CH, CR⁵¹, or N,
Z⁵² is CH, CR⁵², or N,
Z⁵³ is CH, CR⁵³, or N,
Z⁵⁴ is CH, CR⁵⁴, or N,
Z⁵⁵ is CH, CR⁵⁵, or N,
Z⁵⁶ is CH, CR⁵⁶, or N,
Z⁵⁷ is CH, CR⁵⁷, or N,
Z⁵⁸ is CH, CR⁵⁸, or N,
R⁵¹ to R⁵⁸ are each independently any one group selected from (5a) to (5h):
(5a) a cyano group;
(5b) a substituted or unsubstituted alkyl group having 1 to 20 carbon atoms;
(5c) a substituted or unsubstituted alkoxy group having 1 to 20 carbon atoms;
(5d) a substituted or unsubstituted arylamino group having 6 to 20 carbon atoms;
(5e) a substituted or unsubstituted phosphoryl group (a —POH₂ group);
(5f) a substituted or unsubstituted silyl group (a —SiH₃ group);
(5g) a substituted or unsubstituted monovalent aromatic hydrocarbon group having 6 to 30 carbon atoms; and
(5h) a substituted or unsubstituted monovalent heterocyclic group having 3 to 30 ring-forming atoms,
wherein Ar⁵¹ is a group including at least one of an aromatic hydrocarbon group and a heterocyclic group,
m is 1, 2, 3, 4, 5, or 6, and
wherein R⁵¹ and R⁵², R⁵² and R⁵³, R⁵³ and R⁵⁴, R⁵⁵ and R⁵⁶, R⁵⁶ and R⁵⁷, or R⁵⁷ and R⁵⁸ optionally form an aliphatic hydrocarbon ring, an aromatic hydrocarbon ring, or a hetero ring, each including bonded carbon atoms.

17. The organic electroluminescent device of claim 14, wherein the host material comprises a compound having a triazine ring structure.

18. The organic electroluminescent device of claim 14, wherein the host material comprises a compound having a structure represented by Formula (6): 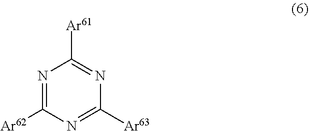
wherein, in Formula (6),
Ar⁶¹ to Ar⁶³ are each independently a substituted or unsubstituted monovalent aromatic hydrocarbon group having 6 to 30 carbon atoms or a substituted or unsubstituted monovalent heterocyclic group having 3 to 30 ring-forming atoms.

19. The organic electroluminescent device of claim 10, wherein the emission layer further comprises a host material and a dopant material.

20. The organic electroluminescent device of claim 19, wherein the compound is the dopant material.
